(19) United States
(12) Patent Application Publication
(10) Pub. No.: US 2007/0093485 A1
(43) Pub. Date: Apr. 26, 2007

(54) Substituted n-phenyl sulfonamide bradykinin antagonists

(75) Inventors: Francine S. Farouz, Mercer Island, WA (US); Michael S. Dappen, Redwood City, CA (US); Ying-Zi Xu, Palo Alto, CA (US); Sarah Bartulis, Berkeley, CA (US); Ryan C. Holcomb, Bellevue, WA (US); Ramesh A. Kasar, Bellevue, WA (US); Michael A. Pleiss, Sunnyvale, CA (US); Eugene D. Thorsett, Kirkland, WA (US); Xiaocong Michael Ye, Palo Alto, CA (US); Andrei W. Konradi, Burlingame, CA (US)

Correspondence Address: FOLEY & LARDNER LLP, 1530 PAGE MILL ROAD, PALO ALTO, CA, 94304, US

(73) Assignee: ELAN PHARMACEUTICALS, INC.

(21) Appl. No.: 10/527,384

(22) PCT Filed: Dec. 18, 2003
(86) PCT No.: PCT/US03/40745

§ 371 Date: Dec. 19, 2006

Related U.S. Application Data
(60) Provisional application No. 60/435,123, filed on Dec. 19, 2002.

(51) Int. Cl. A61K 31/5377; A61K 31/497; A61K 31/4965; A61K 31/496; A61K 31/4545; A61K 31/454; A61K 31/4439; A61K 31/427; A61K 31/22; C07D 417/02; C07D 403/02

(52) U.S. Cl. 514/237.5; 514/317; 514/357; 514/365; 514/326; 514/252.1; 514/408; 514/318; 514/256; 514/252.12; 514/524; 544/162; 544/406; 544/399; 544/360; 514/253.01; 546/194; 546/207; 544/333; 546/278.1; 548/200; 548/202; 544/392; 514/255.01

(57) ABSTRACT

Disclosed are certain substituted N-phenylsulfonamide derivatives and related compounds. These compounds are useful as bradykinin antagonists to relieve adverse symptoms in mammals mediated, at least in part, by bradykinin including pain, inflammation, bronchoconstriction, cerebral edema, etc.

### BACKGROUND OF THE INVENTION

1. Field of the Invention

This invention is directed to certain substituted N-phenylsulfonamide derivatives and related compounds. These compounds are useful as bradykinin antagonists to relieve adverse symptoms in mammals mediated, at least in part, by bradykinin including pain, inflammtion, bronchoconstriction, cerebral edema, etc.

This invention is also directed to pharmaceutical compositions comprising such N-phenylsulfonamide derivatives and related compounds as well as to method using such compounds.

### REFERENCES

The following literature and patent publications are cited in this application as superscript numbers.
¹ J. G. Menke, et al., J. Biol. Chem., 269(34):21583-2158 (1994)
² J. F. Hess, Biochem. Human B₂ Receptor, Biophys. Res. Commun., 184:260-268 (1992)
³ Burch, et al., “Bradykinin Receptor Antagonists”, J. Med. Chem., 30:237-269 (1990).
⁴ Clark, W. G. “Kinins and the Peripheral Central Nervous Systems”, Handbook of Experimental Pharmacology, Vol. XXV: Bradykinin, Kallidin, and Kallikrein. Erdo, E. G. (Ed.), 311-322 (1979).
⁵ Ammons, W. S., et al., “Effects of Intracardiac Bradykinin on T₂-T₅ Medial Spinothalamic Cells”, The American Physiological Society, 0363-6119 (1985).
⁶ Costello, A. H. et al., “Suppression of Carageenan-Induced Hyperalgesia, Hyperthermia and Edema by a Bradykinin Antagonist”, European Journal of Pharmacology, 171:259-263 (1989).
⁷ Laneuville, et al., “Bradykinin Analogue Blocks Bradykinin-induced Inhibition of a Spinal Nociceptive Reflex in the Rat”, European Journal of Pharmacology, 137:281-285 (1987).
⁸ Steranka, et al., “Antinociceptive Effects of Bradykinin Antagonists”, European Journal of Pharmacology, 16:261-262 (1987).
⁹ Steranka, et al., “Bradykinin as a Pain Mediator: Receptors are Localized to Sensory Neurons, and Antagonists have Analgesic Actions”, Neurobiology, 85:3245-3249 (1987).
¹⁰ Whalley, et al., in Naunyn Schmiederberg's Arch. Pharmacol., 336:652-655 (1987).
¹¹ Back, et al., “Determination of Components of the Kallikrein-Kinin System in the Cerebrospinal Fluid of Patients with Various Diseases”, Res. Clin.Stud. Headaches, 3:219-226 (1972).
¹² Ness, et al., “Visceral pain: a Review of Experimental Studies”, Pain, 41:167-234 (1990).
¹³ Aasen, et al., “Plasma kallikrein Activity and Prekallikrein Levels during Endotoxin Shock in Dogs”, Eur. Surg., 10:5062(1977).
¹⁴ Aasen, et al., “Plasma Kallikrein-Kinin System in Septicemia”, Arch. Surg., 118:343-346 (1983).
¹⁵ Katori, et al., “Evidence for the Involvement of a Plasma Kallikrein/Kinin System in the Immediate Hypotension Produced by Endotoxin in Anaesthetized Rats”, Br. J. Pharmacol., 98:1383-1391 (1989).
¹⁶ Marceau, et al., “Pharmacology of Kinins: Their Relevance to Tissue Injury and Inflammation”, Gen. Pharmacol., 14:209-229 (1982).
¹⁷ Weipert, et al., Brit J. Pharm., 94:282-284 (1988).
¹⁸ Haberland, “The Role of Kininogenases, Kinin Formation and Kininogenase Inhibitor in Post Traumatic Shock and Related Conditions”, Klinische Woochen-Schrift, 56:325-331 (1978).
¹⁹ Ellis, et al., “Inhibition of Bradykinin-and Kallikrein-Induced Cerebral Arteriolar Dilation by Specific Bradykinin Antagonist”, Stroke, 18:792-795 (1987).
²⁰ Kamitani, et al., “Evidence for a Possible Role of the Brain Kallikrein-Kinin System in the Modulation of the Cerebral Circulation”, Circ. Res., 57:545-552 (1985).
²¹ Barnes, “Inflammatory Mediator Receptors and Asthma”, Am. Rev. Respir. Dis., 135:S26-S31 (1987).
²² Burch, et al., “Bradykinin Receptor Antagonists”, J. Med. Chem., 30:237-269 (1990).
²³ Fuller, et al., “Brakykinin-induced Bronchoconstriction in Humans”, Am. Rev. Respir. Dis., 135:176-180 (1987).
²⁴ Jin, et al., “Inhibition of Bradykinin-Induced Bronchoconstriction in the Guinea-Pig by a Synthetic B₂ Receptor Antagonist”, Br. J. Pharmacol., 97:598-602 (1989).
²⁵ Polosa, et al., “Contribution of Histamine and Prostanoids to Bronchoconstriction Provoked by Inhaled Bradykinin in Atopic Asthma”, Allergy, 45:174-182 (1990).
²⁶ Baumgarten, et al., “Concentrations of Glandular Kallikrein in Human Nasal Secretions Increase During Experimentally Induced Allergic Rhinitis”, J. Immunology, 137:1323-1328 (1986).
²⁷ Proud, et al., “Nasal Provocation with Bradykinin Induces Symptoms of Rhinitis and a Sore Throat”, Am. Rev. Respir Dis., 137:613-616 (1988).
²⁸ Steward and Vavrek in “Chemistry of Peptide Bradykinin Antagonists” Basic and Chemical Research, R. M. Burch (Ed.), pages 51-96 (1991).
²⁹ Seabrook, et al., Expression of B1 and B2 Bradykinin Receptor mRNA and Their Functional Roles in Sympathetic Ganglia and Sensory Dorsal Root Ganglia Neurons from Wild-type and B2 Receptor Knockout Mice, Neuropharmacology, 36(7):1009-17 (1997)
³⁰ Elguero, et al., Nonconventional Analgesics: Bradykinin Antagonists, An. R. Acad. Farm., 63(1):173-90 (Spa) (1997)
³¹ McManus, U.S. Pat. No. 3,654,275, Quinoxalinecarboxamide Antiinflammatory Agents, issued Apr. 4, 1972
³² Grant, et al., U.S. Provisional Patent Application Ser. No. 60/378,206, Sulfonylquinoxalone Acetamide Derivatives and Related Compounds as Bradykinin Antagonists, filed May 3, 2002

All of the above identified publications are herein incorporated by reference in their entirety to the same extent as if each individual publication was specifically and individually incorporated by reference in its entirety.

### STATE OF THE ART

Bradykinin (BK) is known to be one of the most potent naturally occurring stimulators of C-fiber afferents mediating pain. It also is a potent vasodilator, edema-producing agent, and stimulator of various vascular and non-vascular smooth muscles in tissues such as uterus, gut and bronchiole. The kinin/kininogen activation pathway has also been described as playing a pivotal role in a variety of physiologic and pathophysiologic processes, being one of the first systems to be activated in the inflammatory response and one of the most potent simulators of: (i) phospholipase A₂ and, hence, the generation of prostaglandins and leukotrienes; and (ii) phospholipase C and thus, the release of inositol phosphates and diacylgylcerol. These effects are mediated predominantly via activation of BK receptors of the BK₂ type.

Bradykinin (BK) is a peptide composed of nine amino acids (Arg¹-Pro²-Pro³-Gly⁴-Phe⁵-Ser⁶-Pro⁷-Phe⁸-Arg⁹) (SEQ. ID. NO. 1) which, along with lysyl-BK (kallidin), is released from precursor kininogens by proteases termed kallikreins. Plasma kallikrein circulates as an inactive zymogen, from which active kallikrein is released by Hageman factor. Tissue kallikrein appears to be located predominantly on the outer surface of epithelial cell membranes at sites thought to be involved in transcellular-electrolyte transport.

B2 receptors are receptors for bradykinin and kallidin; they predominate and are normally found in most tissues. B1 receptors are specific for [des-Arg⁹] bradykinin and [des-Arg¹⁰] kallidin. The B1 subtype is induced by inflammatory processes. Bradykinin receptors have been cloned for different species, notably the human B1 receptor (see J. G. Menke et al.¹, and human B2 receptor J. F. Hess²).

The distribution of receptor B1 is very limited since this receptor is only expressed during states of inflammation. Two generations of peptidic antagonists of the B2 receptor have been developed. The second generation has compounds two orders of magnitude more potent as analgesics than first generation compounds and the most important derivative was icatibant. The first non-peptidic antagonist of the B2 receptor, described in 1993, has two phosphonium cations separated by a modified amino acid. Many derivatives of this di-cationic compound have been prepared. Another non-peptidic compound antagonist of B2 is the natural product Martinelline. See Elguero.³⁰ See also Seabrook.²⁹

Two major kinin precursor proteins, high molecular weight and low molecular weight kininogen are synthesized in the liver, circulate in plasma, and are found in secretions such as urine and nasal fluid. High molecular weight kininogen is cleaved by plasma kallikrein, yielding BK, or by tissue kallikrein, yielding kallidin. Low molecular weight kininogen, however, is a substrate only for tissue kallikrein. In addition, some conversion of kallidin to BK may occur inasmuch as the amino terminal lysine residue of kallidin is removed by plasma aminopeptidases. Plasma half-lives for kinins are approximately 15 seconds, with a single passage through the pulmonary vascular bed resulting in 80-90% destruction. The principle catabolic enzyme in vascular beds is the dipeptidyl carboxypeptidase kininase II or angiotensin-converting enzyme (ACE). A slower acting enzyme, kininase I, or carboxypeptidase N, which removes the carboxyl terminal Arg, circulates in plasma in great abundance. This suggests that it may be the more important catabolic enzyme physiologically. Des-Arg⁹-bradykinin as well as des-Arg¹⁰-kallidin formed by kininase I acting on BK or kallidin, respectively, are acting BK₁ receptor agonists, but are relatively inactive at the more abundant BK₂ receptor at which both BK and kallidin are potent agonists.

Direct application of bradykinin to denuded skin or intra-arterial or visceral injection results in the sensation of pain in mammals including humans. Kinin-like materials have been isolated from inflammatory sites produced by a variety of stimuli. In addition, bradykinin receptors have been localized to nociceptive peripheral nerve pathways and BK has been demonstrated to stimulate central fibers mediating pain sensation. Bradykinin has also been shown to be capable of causing hyperalgesia in animal models of pain. See, Burch, et al,³ and Clark, W. G.⁴

These observations have led to considerable attention being focused on the use of BK antagonists as analgesics. A number of studies have demonstrated that bradykinin antagonists are capable of blocking or ameliorating both pain as well as hyperalgesia in mammals including humans. See, Ammons, W. S., et al.⁵, Clark, W. G.⁴, Costello, A. H., et al.⁶, Laneuville, et al.⁷, Steranka, et al.⁸ and Steranka, et al.⁹.

Currently accepted therapeutic approaches to analgesia have significant limitations. While mild to moderate pain can be alleviated with the use of non-steroidal anti-inflammatory drugs and other mild analgesics, severe pain such as that accompanying surgical procedures, burns and severe trauma requires the use of narcotic analgesics. These drugs carry the limitations of abuse potential, physical and psychological dependence, altered mental status and respiratory depression which significantly limit their usefulness.

Prior efforts in the field of BK antagonists indicate that such antagonists can be useful in a variety of roles. These include use in the treatment of burns, perioperative pain, migraine and other forms of pain, shock, central nervous system injury, asthma, rhinitis, premature labor, inflammatory arthritis, inflammatory bowel disease, neuropathic pain, etc. For example, Whalley, et al.¹⁰ has demonstrated that BK antagonists are capable of blocking BK-induced pain in a human blister base model. This suggests that topical application of such antagonists would be capable of inhibiting pain in burned skin, e.g., in severely burned patients that require large doses of narcotics over long periods of time and for the local treatment of relatively minor burns or other forms of local skin injury.

The management of perioperative pain requires the use of adequate doses of narcotic analgesics to alleviate pain while not inducing excessive respiratory depression. Post-operative narcotic-induced hypoventilation predisposes patients to collapse of segments of the lungs, a common cause of post-operative fever, and frequently delays discontinuation of mechanical ventilation. The availability of a potent non-narcotic parenteral analgesic could be a significant addition to the treatment of perioperative pain. While no currently available BK antagonist has the appropriate pharmacodynamic profile to be used for the management of chronic pain, frequent dosing and continuous infusions are already commonly used by anesthesiologists and surgeons in the management of perioperative pain.

Several lines of evidence suggest that the kallikrein/kinin pathway may be involved in the initiation or amplification of vascular reactivity and sterile inflammation in migraine. (See, Back, et al.¹¹). Because of the limited success of both prophylactic and non-narcotic therapeutic regimens for migraine as well as the potential for narcotic dependence in these patients, the use of BK antagonists offers a highly desirable alternative approach to the therapy of migraine.

Bradykinin is produced during tissue injury and can be found in coronary sinus blood after experimental occlusion of the coronary arteries. In addition, when directly injected into the peritoneal cavity, BK produces a visceral type of pain. (See, Ness, et al.¹²). While multiple other mediators are also clearly involved in the production of pain and hyperalgesia in settings other than those described above, it is also believed that antagonists of BK have a place in the alleviation of such forms of pain as well.

Shock related to bacterial infections is a major health problem. It is estimated that 400,000 cases of bacterial sepsis occur in the United States yearly; of those 200,000 progress to shock, and 50% of these patients die. Current therapy is supportive, with some suggestion in recent studies that monoclonal antibodies to Gram-negative endotoxin may have a positive effect on disease outcome. Mortality is still high, even in the face of this specific therapy, and a significant percentage of patients with sepsis are infected with Gram-positive organisms which would not be amenable to anti-endotoxin therapy.

Multiple studies have suggested a role for the kallikrein/kinin system in the production of shock associated with endotoxin. See, Aasen, et al.¹³, Aasen, et al.¹⁴, Katori, et al.¹⁵ and Marceau, et al.¹⁶. Recent studies using newly available BK antagonists have demonstrated in animal models that these compounds can profoundly affect the progress of endotoxic shock. (See, Weipert, et al.¹⁷). Less data is available regarding the role of BK and other mediators in the production of septic shock due to Gram-positive organisms. However, it appears likely that similar mechanisms are involved. Shock secondary to trauma, while frequently due to blood loss, is also accompanied by activation of the kallikrein/kinin system. (See, Haberland¹⁸.)

Numerous studies have also demonstrated significant levels of activity of the kallikrein/kinin system in the brain. Both kallikrein and BK dilate cerebral vessels in animal models of CNS injury. (See Ellis, et al.¹⁹ and Kamitani, et al.²⁰). Bradykinin antagonists have also been shown to reduce cerebral edema in animals after brain trauma. Based on the above, it is believed that BK antagonists should be useful in the management of stroke and head trauma.

Other studies have demonstrated that BK receptors are present in the lung, that BK can cause bronchoconstriction in both animals and man and that a heightened sensitivity to the bronchoconstrictive effect of BK is present in asthmatics. Some studies have been able to demonstrate inhibition of both BK and allergen-induced bronchoconstriction in animal models using BK antagonists. These studies indicate a potential role for the use of BK antagonists as clinical agents in the treatment of asthma. (See Barnes²¹, Burch, et al.²², Fuller, et al.²³, Jin, et al.²⁴ and Polosa, et al.²⁵.) Bradykinin has also been implicated in the production of histamine and prostanoids to bronchoconstriction provoked by inhaled bradykinin in atopic asthma.²⁵ Bradykinin has also been implicated in the production of symptoms in both allergic and viral rhinitis. These studies include the demonstration of both kallikrein and BK in nasal lavage fluids and that levels of these substances correlate well with symptoms of rhinitis. (See, Baumgarten, et al.²⁶, Jin, et al.²⁴, and Proud, et al.²⁷)

In addition, studies have demonstrated that BK itself can cause symptoms of rhinitis. Stewart and Vavrek²⁸ discuss peptide BK antagonists and their use against effects of BK.

A great deal of research effort has been expended towards developing such antagonists with improved properties. However, notwithstanding extensive efforts to find such improved BK antagonists, there remains a need for additional and more effective BK antagonists. Two of the major problems with presently available BK antagonists are their low levels of potency and their extremely short durations of activity. Thus there is a special need for BK antagonists having increased potency and for duration of action.

U.S. Pat. No. 3,654,275³¹ teaches that certain 1,2,3,4-tetrahydro-1-acyl-3-oxo-2-quinoxalinecarboxamides have anti-inflammatory activity and describes the preparation of certain intermediates which can also be used as intermediates in the preparation of the compounds hereafter described.

In addition, U.S. Provisional Patent Application Ser. No. 60/378,206, filed May 3, 2002 discloses a variety of sulfonylquinoxalone acetamide derivatives as BK antagonists.³²

In view of the above, compounds which are bradykinin antagonists would be particularly advantageous in treating those diseases mediated by bradykinin.

### SUMMARY OF THE INVENTION

This invention is directed, in part, to compounds which are bradykinin antagonists and are useful to treat diseases or relieve adverse symptoms associated with disease conditions in mammals mediated at least in part by bradykinin. Certain of the compounds exhibit increased potency and are expected to also exhibit an increased duration of action.

The present invention provides compounds of Formula I: 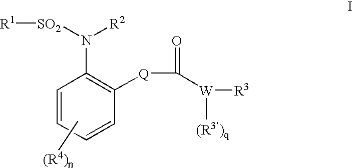
wherein

Q is selected from the group consisting of C₂-C₃ alkylene, C₂-C₃ alkenylene and C₂-C₃ alkynylene;

W is selected from the group consisting of O, S, and N, wherein: when W is O or S, then q is zero; and when W is N, then q is one;

R¹ is selected from the group consisting of aryl, substituted aryl, heteroaryl, substituted heteroaryl, heterocyclic and substituted heterocyclic;

R² is selected from the group consisting of hydrogen, alkyl, cycloalkyl, aryl, aralkyl, heteroaryl, heteroaralkyl and heterocyclic;

R³ and R^{3′} are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic and substituted heterocyclic, or R³ and R^{3′} together with the nitrogen atom to which they are attached form a heteroaryl, substituted heteroaryl, heterocyclic, or substituted heterocyclic;

each R⁴ is independently selected from the group consisting of alkyl, amino, substituted amino, cycloalkyl, alkoxy, aryl, heteroaryl, heterocyclic, acyl, halogen, nitro, cyano, hydroxy, carboxy, —C(O)OR¹⁰ wherein R¹⁰ is alkyl, substituted alkyl, aryl, or substituted aryl, and —C(O)NR¹¹R¹² wherein R¹¹ and R¹² are independently selected from the group consisting of hydrogen, alkyl, aryl, cycloalkyl, heteroaryl, and heterocyclic, or R¹¹ and R¹² together with the nitrogen atom to which they are joined form a heteroaryl, substituted heteroaryl, heterocyclic or a substituted heterocyclic group;

n is an integer of from 0 to 3;

or pharmaceutically acceptable salts, prodrugs or isomers thereof.

Preferred R¹ groups include, for example, phenyl; naphth-1-yl; 5-dimethylaminonaphth-1-yl; 2-fluorophenyl; 2-chlorophenyl; 2-cyanophenyl; 2-methylphenyl; 2-nitrophenyl; 2-trifluoromethylphenyl; 3-chlorophenyl; 4-methylphenyl(tolyl); 2,5-dibromophenyl; 4-bromo-2-ethylphenyl; 4-bromo-2-trifluoromethoxyphenyl; 2,3-dichlorophenyl; 2,4dichlorophenyl; 3,4-dichlorophenyl; 2,5dichlorophenyl; 3,5dichlorophenyl; 2,6-dichlorophenyl; 2-chloro-4-cyanophenyl; 2-chloro-4-fluorophenyl; 3-chloro-2-methylphenyl; 2-chloro-6-methylphenyl; 5-chloro-2-methoxyphenyl; 2-chloro-4-trifluoromethyl-phenyl; 2,4difluorophenyl; 5-fluoro-2-methylphenyl; 2,5-dimethoxyphenyl; 2-methoxymethylphenyl; 2-methoxy-5-bromophenyl; 2-methoxy-5-methyl-phenyl; 2,5-dimethylphenyl; 2-methyl-5-nitrophenyl; 3,5-di(trifluoromethyl)-phenyl; 4-bromo-2,5-difluorophenyl; 2,3,4-trichlorophenyl; 2,4,5-trichlorophenyl; 2,4,6-trichlorophenyl; 2,4-dichloro-5-methylphenyl; 4-chloro-2,5-dimethylphenyl; 2,4,6-tri(iso)propylphenyl; 2,4,6-triethylphenyl; 2,3,5-trimethylchlorophenyl; 2,3,6-trimethyl-4-methoxyphenyl; 2,3,4,5,6-pentamethylphenyl; 5-chloro-1,3-dimethylpyrazol-4-yl; 2-methoxycarbonyl-thiophen-3-yl; 2,3-dimethyl-imidazol-5yl; 2-methylcarbonylamino-4-methyl-thiazol-5-yl; quinolin-8-yl; thiophen-2-yl; 1-methylimidiazol-4-yl; 3,5-dimethylisoxazol-4-yl; N-morpholino; 2,3,4-trifluoro-phenyl; 2,4-dichloro-3-methylphenyl; 2,4-dimethyl-5-chlorophenyl; 2-chloro-5-methylphenyl; 2-methyl-4-fluorophenyl; 2-phenoxyphenyl; 3-(4-methyl-phenoxy)-phenyl; 3,4-difluorophenyl; 3,4-dimethoxyphenyl; 3-chloro-4-fluorophenyl; 3-chloro-4-methylphenyl; 3-methylphenyl; and 6-chloro-5-bromopyrid-3-yl.

Particularly preferred R¹ groups include 4-chloro-2,5-dimethylphenyl and 2,3-dichlorophenyl.

For R² groups which are heteroaryl or heterocyclic, it is understood that these groups are attached to the nitrogen atom of the sulfonamide via a carbon atom. In any event, preferably, R² is hydrogen or alkyl and, more preferably, methyl, ethyl, and the like.

When W is N, preferred R³ groups include, for example,

amino,

2-[N-(α-aminoacetyl)piperidyl]ethyl,

4-aminobenzyl,

2-[N-(1-amino-1-methylethylcarbonyl)piperid-4-yl]ethyl,

2-(4-aminophenyl)ethyl,

2-aminothiazol-5-ylmethyl,

(2-aminopyrid-4-yl)methyl,

benzyl,

2-bromoethyl,

1-(S)-carboxamide-2-(indol-3-yl)ethyl,

carboxamidemethyl,

1-carboxamide-2-(S)-methyl-butyl,

1-(S)-carbamyol-2-(phenyl)ethyl,

1-(R)-carboxamide-2-(phenyl)ethyl,

4-carboxybenzyl,

2-chloroethyl,

cyanomethyl,

2-(4-cyanophenyl)ethyl,

2-(4-cyanophenyl)-1-(R)-(pyrrolidin-N-ylcarbonyl)ethyl,

2-(4-cyanophenyl)-1-(S)-(pyrrolidin-N-ylcarbonyl)ethyl,

cyclohexyl,

cyclohexylmethyl,

2-(N-cyclopropylpiperidin-4-yl)ethyl,

2-(N-cyclopropylpiperidin-4yl)-1-(R)-(pyrrolidin-N-ylcarbonyl)ethyl,

1-(R)-1,3-di(benzyloxycarbonyl)propyl,

1-(S)-1,3-dicarboxamidepropyl,

(2-dimethylamino)ethyl,

2-[4-(N,N-dimethylamino]phenethyl,

3-(dimethylamino)propyl,

1-(S)-ethoxycarbonylethyl,

2-ethoxyphenyl,

ethyl,

1-(R)-(1-N-ethylaminocarbonyl)-4amino-n-butyl,

1-(S)-(1-N-ethylaminocarbonyl)-4-amino-n-butyl,

1-(R)-(1-N-ethylaminocarbonyl)-5-(t-butoxycarbonylamino)pent-5-yl,

1-(S)-(1-N-ethylaminocarbonyl)-5-(t-butoxycarbonylamino)pent-5-yl,

1-(R)-(1-N-ethylaminocarbonyl)-4-(N′-t-butoxycarbonylamino)-n-but-5-yl,

1-(S)-(1-N-ethylaminocarbonyl)-4-(N′-t-butoxycarbonylamino)-n-but-5-yl,

1-(R)-(1-N-ethylaminocarbonyl)-5-(N′-t-butoxycarbonylamino)-n-pent-5-yl

1-(S)-(1-N-ethylaminocarbonyl)-5-N′-t-butoxycarbonylamino)-n-pent-5-yl,

4-fluorophenethyl,

hydrogen,

2-hydroxyethyl,

2-(4-hydroxyphenyl)-1-(S)-(methoxycarbonyl)ethyl,

2-(4-hydroxyphenyl)-1-(S)-(isopropoxycarbonyl)ethyl,

2-(4-hydroxyphenyl)-1-(R)-(methoxycarbonyl)ethyl,

2-(N-hydroxypyrid-4-yl)ethyl,

2-(imidazol-4-yl)ethyl,

2-[4-(imidazolin-2-yl)phenyl]-1-(R)-(pyrrolidin-1-ylcarbonyl)ethyl,

2-[4-(imidazolin-2-yl)phenyl]ethyl,

2-(indol-3-yl)ethyl,

2-(indol-3-yl)-1-(S)-(methoxycarbonyl)ethyl,

2-(indol-3-yl)-1-(R)-(methoxycarbonyl)ethyl,

iso-propyl,

1-(R)-(isopropoxycarbonyl)-2-(phenyl)ethyl,

4-(methoxycarbonyl)benzyl,

1-(R)-(methoxycarbonyl)ethyl,

methoxycarbonylmethyl,

methoxycarbonylphenylmethyl,

2-methoxyethyl,

1-(R)-(methoxcarbonyl)-2-(N-methylpiperidin-4-yl)ethyl,

1-(R)-(methoxycarbonyl)-2-(N-methyl-1,2,3,6-tetrahydropyrid-4-yl)ethyl,

2-methoxyphenyl,

1-(R)-(methoxycarbonyl)-2-pyrid-4-yl)ethyl,

methyl,

2-[4-(methylcarbonylamino]phenethyl,

2-4-methylpiperazin-1-yl)ethyl,

2-(N-methylpiperidin-4-yl)ethyl,

(N-methylpiperidin-2-yl)methyl,

2-(N-methylpiperidin-2-yl)ethyl,

2-(N-methylpiperidin-3-yl)ethyl,

ethyl2-(N-methylpiperidin-4-yl)-1-(R)-(pyrrolidin-N-ylcarbonyl)ethyl,

2-[(N-methyl)pyrrolidin-2-yl]ethyl,

2-N-methyl-1,2,5,6-tetrahydropyrid-4-yl)ethyl,

2-(N-methyl-1,2,5,6-tetrahydropyrid-4yl)-1-(R)-(pyrrolidin-N-ylcarbonyl)ethyl,

3-(2-methylthiazol-5-yl)-pyrazol-5-yl,

2-(N-morpholino)ethyl,

n-hexyl,

4-nitrobenzyl,

phenethyl,

1-(R)-phenylethyl,

1-(S)-phenylethyl,

phenyl,

4-phenylbutyl,

1-(R)-2-phenylcarboxyethyl,

1-(R)-2-phenyl-1-(methoxycarbonyl)ethyl,

1-(S)-2-phenyl-1-(methoxycarbonyl)ethyl,

3-phenyl-n-propylpyl,

2-(phenyl)-1-(S)-(pyrrolidin-N-ylcarbonyl)ethyl,

2-(piperidin-N-yl)ethyl,

2-(piperidin-2-yl)ethyl,

2-(piperidin-3-yl)ethyl,

2-(piperidin-4yl)ethyl,

(piperid-1-yl)carbonylmethyl,

pyrazin-2-ylmethyl,

2-(pyrid-2-yl)ethyl,

2-(pyrid-3-yl)ethyl,

2-(pyrid-4yl-)ethyl,

(pyrid-2-yl)methyl,

(pyrid-3-yl)methyl,

(pyrid-4-yl)methyl,

2-[N-(pyrid-4-yl)]piperidin-4-yl]ethyl,

2-[N-(pyrid-3-yl)piperidin-4yl)]ethyl,

2-[N-(pyrid-2-yl)piperidin-4-yl]ethyl,

2-[N-(4-methylpyrid-2-yl)]piperidin-4-yl]ethyl,

2-[N-(3-methylpyrid-2-yl)]piperidin-4yl]ethyl,

2-(pyrid-4-yl)-1-(R)-(pyrrolidin-N-ylcarbonyl)ethyl,

1-(R)-(pyrrolidin-N-ylcarbonyl)-5-amino-n-pentyl,

1-(S)-(pyrrolidin-N-ylcarbonyl)-5-amino-n-pentyl,

1-(R)-(pyrrolidin-N-ylcarbonyl)-2-(4-biphenyl)ethyl,

1-(S)-(pyrrolidin-N-ylcarbonyl)-2-(4-biphenyl)ethyl,

1-(R)-(pyrrolidin-N-ylcarbonyl-2-(4iodophenyl)ethyl,

1-(S)-(pyrrolidin-N-ylcarbonyl-2-(4-iodophenyl)ethyl,

1-(R)-(pyrrolidin-N-carbonyl)-4-(t-butoxycarbonylamino)-n-butyl,

1-(S)-(pyrrolidin-N-carbonyl)-4-(t-butoxycarbonylamino)-n-butyl,

1-(S)-(pyrrolidin-N-ylcarbonyl)-2-[4-(2-imidazolin-2-yl)phenyl]ethyl,

2-(R)-(pyrrolidin-N-ylcarbonyl-3-phenylprop-2-yl,

1-(R)-(pyrrolidin-N-ylcarbonyl)-2-[4-(N-methylpiperidin-2-yl)phenyl)]ethyl,

1-(S)-(pyrrolidin-N-ylcarbonyl)-2-[4(N-methylpiperidin-2-yl)phenyl)]ethyl,

1-(R)-(pyrrolidin-N-ylcarbonyl)-2-[N-methyl-1,2,5,6-tetrahydro-pyridin-4-yl)-phen-4-yl)]ethyl,

1-(S)-(pyrrolidin-N-ylcarbonyl)-2-[N-methyl-1,2,5,6tetrahydro-pyridin-4-yl)-phen-4-yl)]ethyl,

1-(R)-(pyrrolidin-N-ylcarbonyl)-2-[4-piperidin-2-yl)cyclohexyl)]ethyl,

1-(S)-(pyrrolidin-N-ylcarbonyl)-2-[4-piperidin-2-yl)cyclohexyl)]ethyl,

1-(R)-(pyrrolidin-N-ylcarbonyl)-2-[N-(phenyl)piperidin-4-yl)]ethyl,

1-(S)-(pyrrolidin-N-ylcarbonyl)-2-[N-(phenyl)piperidin-4-yl)]ethyl,

1-(R)-(pyrrolidin-N-ylcarbonyl)-2-[N-(pyridinyl)piperidin-4-yl)]ethyl,

1-(S)-(pyrrolidin-N-ylcarbonyl)-2-[N-(pyridin-4-yl)piperidin-4-yl)]ethyl,

1-(R)-(pyrrolidin-N-ylcarbonyl)-2-[4-(pyridin-4-yl)phenyl)]ethyl,

1-(S)-(pyrrolidin-N-ylcarbonyl)-2-[4-(pyridin-4-yl)phenyl)]ethyl,

1-(R)-(pyrrolidin-N-ylcarbonyl)-2-[2-(pyrid-2-yl)phenyl]ethyl,

1-(S)-(pyrrolidin-N-ylcarbonyl)-2-[4-(pyrid-2-yl)phenyl]ethyl,

1-(R)-(pyrrolidin-N-ylcarbonyl)-2-[4-(pyrimidin-2-yl)phenyl]ethyl,

1-(S)-(pyrrolidin-N-ylcarbonyl)-2-[4-(pyrimidin-2-yl)phenyl]ethyl,

1-(R)-(pyrrolidin-N-ylcarbonyl)-2-[4-(N-t-butoxycarbonylpyrrol-2-yl)phenyl]ethyl,

1-(S)-(pyrrolidin-N-ylcarbonyl)-2-[4-(N-t-butoxycarbonylpyrrol-2-yl)phenyl]ethyl,

1(S)-(t-butoxycarbonyl)-2-(4-hydroxyphenyl)ethyl,

3-t-butoxycarbonyl-1-methoxycarbonylpropyl,

2-[N-(t-butoxycarbonylmethyl)piperid-4-yl]ethyl,

2-[1-(t-butoxycarbonylmethyl)piperid-4-yl)]ethyl,

1-S)-(t-butoxycarbonyl)-3-methylpropyl,

1-(R)-(t-butoxycarbonyl)-3-methylpropyl,

1-(R)-(t-butoxycarbonyl)-2-(phenyl)ethyl,

2-(N-t-butoxycarbonylmethyl)pyridin-4-yl-ethyl,

1-R-(N-pyrrolidinylcarbon-yl)-2-(4-pyridyl)ethyl,

1-S-(N-pyrrolidinylcarbon-yl)-2-(4-pyridyl)ethyl,

1-R-1-(N-piperidinylcarbonyl)ethyl,

1-S-1-(N-piperidinylcarbonyl)ethyl,

1-R-1-methyl-2-(N-piperidinyl)ethyl,

1-S-1-methyl-2-(N-piperidinyl)ethyl,

1-R-1-methyl-2-(4-methylpiperazin-1-yl)ethyl,

1-S-1-methyl-2-(4-methylpiperazin-1-yl)ethyl,

-methoxycarbonylbenzyl,

1(R)-1-[4-methylpiperazinylcarbonyl]ethyl,

1(S)-1-[4-methylpiperazinylcarbonyl]ethyl,

2-(N-(5-methyl-pyrimidin-4-yl)-piperidin-4-yl)ethyl,

2-(N-(pyrimidin-4-yl)-piperidin-4-yl)ethyl,

2-(N,N-dimethylpiperidin-4-yl)ethyl,

2-[4-(piperidinylmethyl)phenyl]ethyl,

2-oxo-5-phenyl-2,3-dihydro-1H-benzo[e][1,4]diazepin-3-yl, and

4-[1-(pyrid-4-yl)-piperazin-4-yl]phenyl.

When W is N, preferred R^{3′} groups include hydrogen, methyl, ethyl, iso-propyl, 2-methoxyethyl, pyrid-3-ylmethyl, and 2-(N,N-dimethylpiperidin-4-yl)ethyl.

In another preferred embodiment, W is N and R³ and R^{3′} are joined, together with the nitrogen atom to which they are bound, to form an optionally substituted heterocyclic including, for example, 4-(2-aminoethyl)-piperidin-1-yl; 4-[2-(N-t-butoxycarbonylamino)ethyl]piperidin-1-yl; 1-(pyridin-2-yl)piperazin-4-yl; N-morpholino; 2-methylpiperid-N-yl; 2-(S)-carboxamide-pyrrolidin-N-yl; 2-(R)-hydroxy-5-(S)-methoxycarbonyl-pyrrolidin-N-yl; 2-(R)-methoxycarbonyl-pyrrolidin-N-yl; 2-(S)-methoxy-methylpyrrolidin-1-yl; 3-(R)-(t-butoxycarbox-amido)pyrrolidin-N-yl; 3 carboxamidepiperd-N-yl; 3-hydroxypyrrolidin-N-yl; 4-acetylpiperazin-1-yl; 4-hydroxypiperid-N-yl; and 4-methylpiperazin-1-yl.

A particularly preferred R^{3′} group is hydrogen.

Preferred R⁴ groups include, for example, chloro, fluoro and methyl.

Preferably, n is zero or 2. Most preferably, n is zero (i.e., all of the R⁴ groups are hydrogen).

Q is preferably ethylene, propylene, ethenylene, propenylene, ethynylene, or propynylene. In addition, Q may be optionally substituted with a methyl or trifluoromethyl group.

In one preferred embodiment, R² is hydrogen, methyl, or ethyl; Q is ethylene or propylene; W is nitrogen; n is zero, 1 or 2; q is 1; R⁸ is methyl or hydrogen; R⁵, R⁶ and R⁷ are independently selected from hydrogen, fluoro, and chloro; and R^{3′} is hydrogen, methyl, ethyl, or isopropyl. Such compounds are represented by formula II as follows: 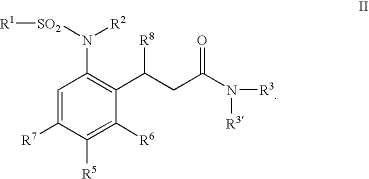
wherein R¹ and R³ are as defined above; and pharmaceutically acceptable salts thereof.

In one preferred embodiment, R² is methyl, Q is ethylene, W is nitrogen, n is zero (all R⁴ groups are hydrogen), q is one and R^{3′} is hydrogen. Such compounds are represented by formula IIa as follows: 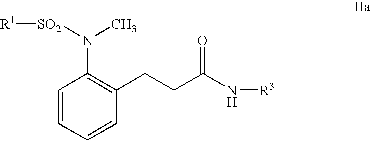
wherein R¹ and R³ are as defined above; and pharmaceutically acceptable salts thereof.

In another particularly preferred embodiment, R² is methyl or ethyl; Q is ethenylene; R⁸ is hydrogen or trifluoromethyl; W is nitrogen; n is zero (all R⁴ groups are hydrogen); q is one; and R^{3′} is hydrogen or methyl. Such compounds are represented by formula III as follows:

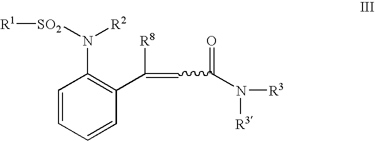

(including both cis and trans isomers) wherein R¹ and R³ are as defined above; and pharmaceutically acceptable salts thereof.

In another embodiment, R² is methyl, Q is ethenylene, W is nitrogen, n is zero (all R⁴ groups are hydrogen), q is one and R^{3′} is hydrogen. Such compounds are represented by formula IIIa as follows: 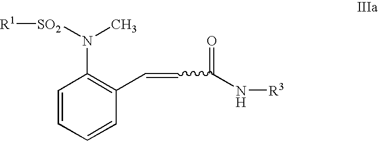
(including both cis and trans isomers) wherein R¹ and R³ are as defined above; and pharmaceutically acceptable salts thereof.

In another embodiment, R² is methyl, Q is ethynylene, W is nitrogen, n is zero, q is one and R^{3′} is hydrogen or methyl. Such compounds are represented by formula IV as follows: 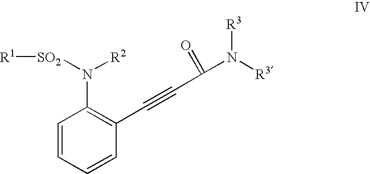
wherein R¹ and R³ are as defined above; and pharmaceutically acceptable salts thereof.

In another embodiment, R² is methyl, Q is ethynylene, W is nitrogen, n is zero, q is one and R^{3′} is hydrogen. Such compounds are represented by formula IV as follows: 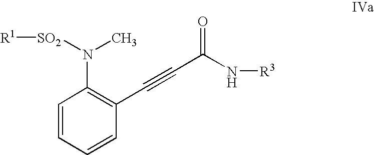
wherein R¹ and R³ are as defined above; and pharmaceutically acceptable salts thereof.

In another particularly preferred embodiment, R² is methyl, Q is propylene, W is nitrogen, n is zero, q is one and R^{3′} is hydrogen or methyl. Such compounds are represented by formula V as follows: 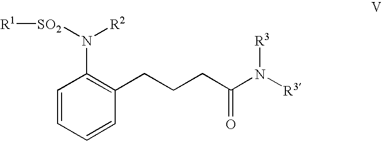
wherein R¹ and R³ are as defined above; and pharmaceutically acceptable salts thereof.

In another embodiment, R² is methyl, Q is propylene, W is nitrogen, n is zero, q is one and R^{3′} is hydrogen. Such compounds are represented by formula Va as follows:

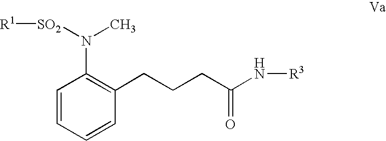

In those cases where the compounds of Formulas I-V exist as optical or geometric isomers, the above formulas are intended to represent isomer mixtures and also the individual BK antagonist.

In the cases of Formula II, the compounds exist as positional cis- or trans-isomers and Formula II is intended to represent both mixtures as well as the individual BK antagonist.

Compounds within the scope of this invention include those set forth in Tables I-IV (where in Table I, Q is ethylene; in Table II, Q is ethenylene; in Table m, Q is ethynylene; and in Table IV, Q is propylene) as follows:

TABLE I

| | |
|---|---|
| | II |
| 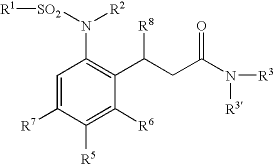 | |

(R^{3′} R⁵, R⁶, R⁷, R⁸ and are hydrogen unless otherwise noted)

| Ex # | R¹ | R⁵, R⁶, R⁷, R⁸ | R² | R³/R^{3′} |
|---|---|---|---|---|
| 1 | 2,5-dimethyl-4-chlorophenyl | | —CH₃ | 1-(R)-(N-pyrrolidinylcarbon-yl)-2-(4-py- |
| | | | | ridyl)ethyl |
| 2 | 2,5-dimethyl-4-chlorophenyl | | —CH₃ | 2-(N-methylpiperidin-4-yl)ethyl |
| 3 | 2,5-dimethyl-4-chlorophenyl | | —CH₃ | 1-(R)-1-(N-piperidinylcar-bonyl)ethyl |
| 4 | 2,5-dimethyl-4-chlorophenyl | | —CH₃ | 1-(S)-1-(N-piperidinylcar-bonyl)ethyl |
| 5 | 2,5-dimethyl-4-chlorophenyl | | —CH₃ | 2-[N-(4-methylpyrid-2-yl)piperidin-4-yl]ethyl |
| 6 | 2,5-dimethyl-4-chlorophenyl | | —CH₃ | 1-(R)-(N-pyrrolidinylcarbon-yl)-2-[(4-py- |
| | | | | ridyl)phen-4-yl]ethyl |
| 7 | 2,5-dimethyl-4-chlorophenyl | | —CH₃ | 2-(N-piperidinyl)ethyl |
| 8 | 2,5-dimethyl-4-chlorophenyl | | —CH₃ | 2-(4-pyridyl)ethyl |
| 9 | 2,5-dimethyl-4-chlorophenyl | | —CH₃ | 2-[N-(2-pyridyl)piperidin-4-yl]ethyl |
| 10 | 2,5-dimethyl-4-chlorophenyl | | —CH₃ | 2-[N-(ethyl)piperidin-4-yl]ethyl |
| 11 | 2,5-dimethyl-4-chlorophenyl | | —CH₃ | 1-(S)-1-methyl-2-(N-piperidinyl)ethyl |
| 12 | 2,5-dimethyl-4-chlorophenyl | | —CH₃ | 2-{N″-(pyrid-4-yl)piperidin-4-yl}ethyl |
| 13 | 2,5-dimethyl-4-chlorophenyl | | —CH₃ | 1-(R)-1-methyl-2-(N-piperidinyl)ethyl |
| 14 | 2,5-dimethyl-4-chlorophenyl | | —CH₃ | 2-[N-(3-methylpyrid-2-yl)piperidin-4-yl]ethyl |
| 15 | 2,5-dimethyl-4-chlorophenyl | | —CH₃ | 1-(S)-1-methyl-2-(4-methylpiperazin-1-yl)ethyl |
| 16 | 2,5-dimethyl-4-chlorophenyl | | —CH₃ | 1-(R)-1-methyl-2-(4-methylpiperazin-1-yl)ethyl |
| 17 | 2,5-dimethyl-4-chlorophenyl | | —CH₃ | R^{3′} = —CH₃ |
| | | | | R³ = 2-(N-methyl-piperidin-4-yl)ethyl |
| 18 | 2,3-dichloro-phenyl | | —CH₃ | R^{3′} = —CH₃ |
| | | | | R³ = 2-(N-methyl-piperidin-4-yl)ethyl |
| 19 | 2,5-dimethyl-4-chlorophenyl | | —CH₃ | —(R,S)-methoxycarbonylbenzyl |
| 20 | 3,4-dichloro-phenyl | | —CH₃ | —(R,S)-methoxycarbonylbenzyl |
| 21 | 2,5-dimethyl-4-chlorophenyl | | —C₂H₅ | 2-(N-ethylpiperidin-4-yl)ethyl |
| 66 | 2,5-dimethyl-4-chlorophenyl | | H | 2-(N-methylpiperidin-4-yl)ethyl |
| 67 | 2,3-dichlorophenyl | | H | 2-(N-methylpiperidin-4-yl)ethyl |
| 68 | 2,5-dimethyl-4-chlorophenyl | | CH₃ | 1(R or S)-1-[4-eth- |
| | | | | ylpiperazinylcarbonyl]ethyl |
| 69 | 2,5-dimethyl-4-chlorophenyl | | CH₃ | 1(S or R)-1-[4-methyl |
| | | | | piperazinylcarbonyl]ethyl |
| 70 | 2,5-dimethyl-4-chlorophenyl | | CH₃ | 2-(N-(3-methylpyrid-2-yl)-piperidin-4-yl)ethyl |
| 71 | 2,5-dimethyl-4-chlorophenyl | | CH₃ | 2-(N,N-dimethylpiperidin-4-yl)ethyl |
| 72 | 2,5-dimethyl-4-chlorophenyl | R⁶ and | H | 2-(N-methylpiperidin-4-yl)ethyl |
| | | R⁷ = Cl | | R^{3′} = methyl |
| 73 | 2,4-dichloro-3-methylphenyl | R⁶ and | H | 2-(N-methylpiperidin-4-yl)ethyl |
| | | R⁷ = Cl | | R3′ = methyl |
| 74 | 2,5-dimethyl-4-chlorophenyl | | CH₂CH₃ | 2-(N-(pyrid-4-yl)-piperidin-4-yl)ethyl |
| | | | | R3′ = methyl |
| 75 | 3-(4-methylphenoxy)-phenyl | | CH₃ | 2-(N-methylpiperidin-4-yl)ethyl |
| 76 | 2,5-dimethyl-4-chlorophenyl | | CH₂CH₃ | 2-(N-(pyrid-4-yl)-piperidin-4-yl)ethyl |
| | | | | R3′ = ethyl |
| 77 | 2,5-dimethyl-4-chlorophenyl | | CH₂CH₃ | 2-oxo-5-phenyl-2,3-dihydro-1H-ben- |
| | | | | zo[e][1,4]diazepin-3-yl |
| 78 | 2,5-dimethyl-4-chlorophenyl | R⁵ and | H | 2-(N-methylpiperidin-4-yl)ethyl |
| | | R⁶ = Cl | | R3′ = methyl |
| 79 | 2,5-dimethyl-4-chlorophenyl | R⁶ and | CH₃ | 2-(N-methylpiperidin-4-yl)ethyl |
| | | R⁷ = Cl | | |
| 80 | 2,5-dimethyl-4-chlorophenyl | R⁵ and | H | 2-(N-methylpiperidin-4-yl)ethyl |
| | | R⁶ = F | | R3′ = methyl |
| 81 | 2,5-dimethyl-4-chlorophenyl | | CH₂CH₃ | 2-(N-methylpiperidin-4-yl)ethyl |
| | | | | R3′ = isopropyl |
| 82 | 2,5-dimethyl-4-chlorophenyl | R⁵ and | CH₃ | 2-(N-methylpiperidin-4-yl)ethyl |
| | | R⁶ = F | | R3′ = methyl |
| 83 | 2,5-dimethyl-4-chlorophenyl | R⁸ = | CH₃ | 2-(N-(pyrid-4-yl)-piperidin-4-yl)ethyl |
| | | CH₃ | | |
| 84 | 2,5-dimethyl-4-chlorophenyl | R⁸ = | CH₃ | 2-(N-(pyrid-4-yl)-piperidin-4-yl)ethyl |
| | | CH₃ | | |
| 85 | 2,5-dimethyl-4-chlorophenyl | R⁸ = | CH₃ | 2-(N-(pyrid-4-yl)-piperidin-4-yl)ethyl |
| | | CH₃ | | |
| 86 | 4-methylphenyl | | H | 2-(N,N-dimethylpiperidin-4-yl)ethyl |
| 87 | 3-chloro-4-methylphenyl | | H | 2-(N,N-dimethylpiperidin-4-yl)ethyl |
| 88 | 3-chloro-2-methylphenyl | | H | 2-(N,N-dimethylpiperidin-4-yl)ethyl |
| 89 | 3,4-dichlorophenyl | | H | 2-(N,N-dimethylpiperidin-4-yl)ethyl |
| 90 | 2-fluorophenyl | | H | 2-(N,N-dimethylpiperidin-4-yl)ethyl |
| 91 | 2,5-dimethyl-4-chlorophenyl | | H | 2-(N-methylpiperidin-4-yl)ethyl |
| 92 | 2,5-dimethyl-4-chlorophenyl | | H | 2-(N,N-dimethylpiperidin-4-yl)ethyl |
| 93 | 2,5-dimethyl-4-chlorophenyl | | CH₃ | 2-(N-(4-methylpyrid-2-yl)-piperidin-4-yl)ethyl |
| 94 | 2,4-dichlorophenyl | | CH₃ | 2-(N,N-dimethylpiperidin-4-yl)ethyl |
| 95 | 2,5-dimethyl-4-chlorophenyl | | CH₂CH₃ | 2-(N-methylpiperidin-4-yl)ethyl |
| 96 | 2,5-dimethyl-4-chlorophenyl | | CH₂CH₃ | 2-(N-methylpiperidin-2-yl)ethyl |
| 97 | 2,5-dimethyl-4-chlorophenyl | | CH₂CH₃ | 2-(N-methylpiperidin-3-yl)ethyl |
| 98 | phenyl | | CH₃ | 2-(N,N-dimethylpiperidin-4-yl)ethyl |
| 99 | 2,3-dichlorophenyl | | CH₃ | 2-(N,N-dimethylpiperidin-4-yl)ethyl |
| 100 | 2-chlorophenyl | | CH₃ | 2-(N,N-dimethylpiperidin-4-yl)ethyl |
| 101 | 2-methylphenyl | | CH₃ | 2-(N,N-dimethylpiperidin-4-yl)ethyl |
| 102 | 3-chlorophenyl | | CH₃ | 2-(N,N-dimethylpiperidin-4-yl)ethyl |
| 103 | 3-methylphenyl | | CH₃ | 2-(N,N-dimethylpiperidin-4-yl)ethyl |
| 104 | 2,4,5-trichlorophenyl | | CH₃ | 2-(N,N-dimethylpiperidin-4-yl)ethyl |
| 105 | 2,4-dimethyl-5-chlorophenyl | | CH₃ | 2-(N,N-dimethylpiperidin-4-yl)ethyl |
| 106 | 2,5-dimethylphenyl | | CH₃ | 2-(N,N-dimethylpiperidin-4-yl)ethyl |
| 107 | 2,5-dichlorophenyl | | CH₃ | 2-(N,N-dimethylpiperidin-4-yl)ethyl |
| 108 | 2,6-dichlorophenyl | | CH₃ | 2-(N,N-dimethylpiperidin-4-yl)ethyl |
| 109 | 2-methoxy-5-chlorophenyl | | CH₃ | 2-(N,N-dimethylpiperidin-4-yl)ethyl |
| 110 | 2-methyl-5-fluorophenyl | | CH₃ | 2-(N,N-dimethylpiperidin-4-yl)ethyl |
| 111 | 2-chloro-5-methylphenyl | | CH₃ | 2-(N,N-dimethylpiperidin-4-yl)ethyl |
| 112 | 3-fluoro-4-methylphenyl | | CH₃ | 2-(N,N-dimethylpiperidin-4-yl)ethyl |
| 113 | naphthyl | | CH₃ | 2-(N,N-dimethylpiperidin-4-yl)ethyl |
| 114 | 3,4-difluorophenyl | | CH₃ | 2-(N,N-dimethylpiperidin-4-yl)ethyl |
| 115 | 3-chloro-4-fluorophenyl | | CH₃ | 2-(N,N-dimethylpiperidin-4-yl)ethyl |
| 116 | 3,4-dimethoxyphenyl | | CH₃ | 2-(N,N-dimethylpiperidin-4-yl)ethyl |
| 117 | 2-chloro-4-cyanophenyl | | CH₃ | 2-(N,N-dimethylpiperidin-4-yl)ethyl |
| 118 | 2,5-dimethyl-4-chlorophenyl | | CH₃ | 2-(N,N-dimethylpiperidin-4-yl)ethyl |
| 119 | 2,5-dimethyl-4-chlorophenyl | | CH₃ | 2-(N-(4-methylpyrid-2-yl)-piperidin-4-yl)ethyl |
| | | | | R3′ = methyl |
| 120 | 2,4,6-trimethylphenyl | | CH₃ | 2-(N,N-dimethylpiperidin-4-yl)ethyl |
| 121 | naphthyl | | CH₃ | 2-(N,N-dimethylpiperidin-4-yl)ethyl |
| 122 | 4-chlorobenzo[c][1,2,5]oxa- | | CH₃ | 2-(N,N-dimethylpiperidin-4-yl)ethyl |
| | diazol-7-yl | | | |
| 123 | 2-phenoxyphenyl | | CH₃ | 2-(N,N-dimethylpiperidin-4-yl)ethyl |
| 124 | 2,3,4-trifluorophenyl | | CH₃ | 2-(N,N-dimethylpiperidin-4-yl)ethyl |
| 125 | 2-chloro-4-(tri- | | CH₃ | 2-(N,N-dimethylpiperidin-4-yl)ethyl |
| | fluoromethyl)phenyl | | | |
| 126 | 2-methyl-4-fluorophenyl | | CH₃ | 2-(N,N-dimethylpiperidin-4-yl)ethyl |
| 127 | 6-chloro-5-bromopyrid-3-yl | | CH₃ | 2-(N,N-dimethylpiperidin-4-yl)ethyl |
| 128 | 3,5-dichlorophenyl | | CH₃ | 2-(N,N-dimethylpiperidin-4-yl)ethyl |
| 129 | 3,5-dichlorophenyl | | CH₃ | 2-(N,N-dimethylpiperidin-4-yl)ethyl |
| 130 | 2,5-dimethyl-4-chlorophenyl | | CH₂CH₃ | 2-(N-(pyrimidin-4-yl)-piperidin-4-yl)ethyl |
| 131 | 2,5-dimethyl-4-chlorophenyl | | CH₂CH₃ | 2-(N-(5-methyl-pyrimidin-4-yl)-pipe- |
| | | | | ridin-4-yl)ethyl |
| 132 | 2,5-dimethyl-4-chlorophenyl | | CH₃ | 2-[4-(piperidinylmethyl)phenyl]ethyl |
| 133 | 2,5-dimethyl-4-chlorophenyl | | CH₃ | |
| 134 | 2,6-dichlorophenyl | | CH₂CH₃ | 2-(N-methylpiperidin-4-yl)ethyl |

Particularly preferred compounds include the following compounds and pharmaceutically acceptable salts thereof:

3-[2′-{(4″-chloro-2″, 5″-dimethylbenzene)-N′-methylsulfonamido}-phenyl]-N-[1-(R)-1-(N″-pyrrolidinylcarbonyl)-2-(4-pyridyl)eth-1-yl]propionamide (1);

3-[2′-{(4″-chloro-2″,5″-dimethylbenzene)-N′-methylsulfonamido}-phenyl]-N-[2-(N″-methylpiperidin-4-yl)eth-1-yl]propionamide (2);

3-[2′-{(4″-chloro-2″,5″-dimethylbenzene)-N′-methylsulfonamido}-phenyl]-N-[1-(R)-1-(N″-piperidinylcarbonyl]eth-1-yl]propionamide (3);

3-[2′-{(4″-chloro-2″,5″-dimethylbenzene)-N′-methylsulfonamido}-phenyl]-N-[1-(S)-1-(N″-piperidinylcarbonyl]eth-1-yl]propionamide (4);

3-[2′-{(4″-chloro-2″,5″-dimethylbenzene)-N′-methylsulfonamido}-phenyl]-N-[2-{N″-(4-methylpyrid-2-yl)}piperidin-4-yl]eth-1-yl]propion-amide (5);

3-[2′-{(4″-chloro-2″,5″-dimethylbenzene)-N′-methylsulfonamido}-phenyl]-N-[1-(R)-1-(N″-pyrrolidinylcarbonyl)-2-(4-pyridylphen-4-yl)eth-1-yl]propionamide (6);

3-[2′-{(4″-chloro-2″,5″-dimethylbenzene)-N′-methylsulfonamido}-phenyl]-N-[(2-N″-piperidinyl)eth-1-yl]propionamide (7);

3-[2′-{(4″-chloro-2″,5″-dimethylbenzene)-N′-methylsulfonamido}-phenyl]-N-[(2-pyrid-4-yl)eth-1-yl]propionamide (8);

3-[2′-{(4″-chloro-2″,5″-dimethylbenzene)-N′-methylsulfonamido}-phenyl]-N-[2-{N″-{(2-pyridyl)piperidin-4-yl}eth-1-yl]propionamide (9);

3-[2′-{(4″-chloro-2″,5″-dimethylbenzene)-N′-methylsulfonamido}-phenyl]-N-[2-(N″-ethylpiperidin-4-yl)eth-1-yl]propionamide (10);

3-[2′-{(4″-chloro-2″,5″-dimethylbenzene)-N′-methylsulfonamido}-phenyl]-N-[1-(S)-1-methyl-2-(N″-piperidinyl)eth-1-yl]propionamide (11);

3-[2′-{(4″-chloro-2″,5″-dimethylbenzene)-N′-methylsulfonamido}-phenyl]-N-[2-{N″-(pyrid-4-yl}piperidin-4-yl)eth-1-yl]propionamide (12);

3-[2′-{(4″-chloro-2″,5″-dimethylbenzene)-N′-methylsulfonamido}-phenyl]-N-[1-(R)-1-methyl-2-(N″-piperidinyl)eth-1-yl]propionamide (13);

3-[2′-{(4″-chloro-2″,5″-dimethylbenzene)-N′-methylsulfonamido}-phenyl]-N-[2-{N″-(3-methylpyrid-2-yl}piperidin-4-yl)eth-1-yl]propion-amide (14);

3-[2′-{(4″-chloro-2″,5″-dimethylbenzene)-N′-methylsulfonamido}-phenyl]-N-[1-(S)-1-methyl-2-(4-methylpiperazin-1-yl)eth-1-yl]propion-amide (15);

3-[2′-{(4″-chloro-2″,5″-dimethylbenzene)-N′-methylsulfonamido}-phenyl]-N-[1-(R)-1-methyl-2-(4-methylpiperazin-1-yl)eth-1-yl]propionamide (16);

3-[2′-{(4″-chloro-2″,5″-dimethylbenzene)-N′-methylsulfonamido}-phenyl]-N-methyl-N-2-(N″-methylpiperidin-4-yl)eth-1-yl]propionamide (17);

3-[2′-{(2″,3″-dichlorobenzene)-N′-methylsulfonamido}phenyl]-N-methyl-N-2-[(N″-methylpiperidin-4-yl)eth-1-yl]propionamide (18);

3-[2′-{(2″,5″-dimethyl-4″-chlorobenzene)-N′-methylsulfonamido}-phenyl]-N-[(α-(R,S)-methoxycarbonyl)benzyl]propionamide (19);

3-[2′-{(2″,3″-dichlorobenzene)-N′-methylsulfonamido}phenyl]-N-N-[(α-(R,S)-methoxycarbonyl)benzyl]propionamide (20);

3-[2′-{(4″-chloro-2″,5″-dimethylbenzene)-N′-ethylsulfonamido}-phenyl]-N-[2-(N″-ethylpiperdin-4-yl)eth-1-yl]propionamide (21);

3-[2′-{(4″-chloro-2″,5″-dimethylbenzene)sulfonamido}-phenyl]-N-[2-(N″-methylpiperidin-4-yl)ethyl]propionamide (66);

3-[2′-{(2″,3″-dichlorobenzene)sulfonamido}-phenyl]-N-[2-(N″-methylpiperidin-4-yl)ethyl]propionamide (67);

3-[2′-{(4″-chloro-2″,5″-dimethylbenzene)-N′-methylsulfonamido}-phenyl]-N-[1(R or S)-1-(4-methyl piperazinylcarbonyl)ethyl]propion-amide (68);

3-[2′-{(4″-chloro-2″,5″-dimethylbenzene)-N′-methylsulfonamido}-phenyl-]-N-[1(S or R)-1-(4-methyl piperazinylcarbonyl)ethyl]propion-amide (69);

3-[2′-{(4″-chloro-2″,5″-dimethylbenzene)-N′-methylsulfonamido}-phenyl]-N-[2-{N″-(3-methylpyrid-2-yl)}piperidin-4yl]ethyl]propion-amide (70);

3-[2′-{(4″-chloro-2″,5″-dimethylbenzene)-N′-methylsulfonamido}-phenyl]-N-[2-(N″,N″-dimethylpiperidin-4-yl)ethyl]propionamide (71);

3-[2′-{(4″-chloro-2″,5″-dimethylbenzene)sulfonamido}-4,5-dichlorophenyl]-N-methyl-N-[2-(N″-methylpiperidin-4-yl)ethyl]propionamide (72);

3-[2′-{(3″-methyl-2″,4″-dichlorobenzene)sulfonamido}-4,5-dichlorophenyl]-N-methyl-N-[2-(N″-methylpiperidin-4-yl)ethyl]propionamide (73);

3-[2′-{(4″-chloro-2″,5″-dimethylbenzene)-N′-ethylsulfonamido}phenyl]-N-methyl-N-[2-[N″-(pyrid-2-yl)piperidin-4-yl]ethyl]propionamide (74);

3-[2′-{(3″-(4″″-methylphenoxy)benzene)-N′-methylsulfonamido}phenyl]-N-[2-(N″-methylpiperidin-4-yl)ethyl]propionamide (75);

3-[2′-{(4″-chloro-2″,5″-dimethylbenzene)-N′-ethylsulfonamido}phenyl]-N-ethyl-N-[2-[N″-(pyrid-2-yl)piperidin-4-yl]ethyl]propionamide (76);

3-[2′-{(4″-chloro-2″,5″-dimethylbenzene)-N′-ethylsulfonamido}phenyl]-N-[2-oxo-5-phenyl-2,3-dihydro-1H-benzo[e][1,4]diazepin-3-yl]propionamide (77);

3-[2′-{(4″-chloro-2″,5″-dimethylbenzene)-N′-methylsulfonamido}-5,6-dichlorophenyl]-N-methyl-N-[2-(N″-methylpiperidin-4-yl)ethyl]propionamide (78);

3-[2′-{(4″-chloro-2″,5″-dimethylbenzene)-N′-methylsulfonamido}-4,5-dichlorophenyl]-N-[2-(N″-methylpiperidin-4-yl)ethyl]propionamide (79);

3-[2′-{(4″-chloro-2″,5″-dimethylbenzene)-N′-methylsulfonamido}-5,6-difluorophenyl]-N-methyl-N-[2-(N″-methylpiperidin-4-yl)ethyl]propionamide (80);

3-[2′-{(4″-chloro-2″,5″-dimethylbenzene)-N′-ethylsulfonamido}phenyl]-N-isopropyl-N-]2-(N″-methylpiperidin-4-yl)ethyl]propionamide (81);

3-[2′-{(4″-chloro-2″,5″-dimethylbenzene)-N′-methylsulfonamido}-5,6-difluorophenyl]-N-methyl-N-[2-(N″-methylpiperidin-4-yl)ethyl]propionamide (82);

3-methyl-3-[2′-{(4″-chloro-2″,5″-dimethylbenzene)-N′-methylsulfonamido)-phenyl]-N-[2-{N″-(pyrid-4-yl}piperidin-4-yl)ethyl]propionamide(Racemic mixture) (83);

3-methyl-3-[2′-{(4″-chloro-2″,5″dimethylbenzene)-N′-methylsulfonamido}-phenyl]-N-[2-{N″-(pyrid-4-yl}piperidin-4-yl)ethyl]propionamide (Isomer A, of racemic mixture) (84);

3-methyl-3-[2′-{(4″-chloro-2″,5″-dimethylbenzene)-N′-methylsulfonamido}-phenyl]-N-[2-{N″-(pyrid-4-yl}piperidin-4-yl)ethyl]propionamide (Isomer B, of racemic mixture) (85);

3-[2′-{(4″-methylbenzene)sulfonamido}-phenyl]-N-[2-(N″,N″-dimethylpiperidin-4-yl)ethyl]propionamide (86);

3-[2′-{(3″-chloro-4″-methylbenzene)sulfonamido}-phenyl]-N-[2-(N″,N″-dimethylpiperidin-4-yl)ethyl]propionamide (87);

3-[2′-{(2″-methyl-3″-chlorobenzene)sulfonamido}-phenyl]-N-[2-(N″,N″-dimethylpiperidin-4-yl)ethyl]propionamide (88);

3-[2′-{(3″,4″-dichlorobenzene)-N′-methylsulfonamido}-phenyl]-N-[2-(N″,N″-dimethylpiperidin-4-yl)ethyl]propionamide (89);

3-[2′-{(2″-fluorobenzene)sulfonamido}-phenyl]-N-[2-(N″,N″-dimethylpiperidin-4-yl)ethyl]propionamide (90);

3-methyl-3-[2′-{(4″-chloro-2″,5″-dimethylbenzene)-N′-methylsulfonamido}-phenyl]-N-[2-(N″-methylpiperidin-4-yl)ethyl]propionamide (91);

3-[2-{(4″-chloro-2″,5″-dimethylbenzene)sulfonamido}-phenyl]-N-[2-(N″,N″-dimethylpiperidin-4-yl)ethyl]propionamide (92);

3-methyl-3-[2′-{(4″-chloro-2″,5″-dimethylbenzene)-N′-methylsulfonamido}-phenyl]-N-[2-{N″-(5-methylpyrid-2-yl)}piperidin-4-yl]ethyl]propionamide (93);

3-[2′-{(2″,4″-dichlorobenzene)-N′-methylsulfonamido}-phenyl]-N-[2-(N″,N″-dimethylpiperidin-4-yl)ethyl]propionamide (94);

3-[2′-{(4″-chloro-2″,5″-dimethylbenzene)-N′-ethylsulfonamido}-phenyl]-N-[2-(N″-methylpiperidin-4-yl)ethyl]propionamide (95);

3-[2′-{(4″-chloro-2″,5″-dimethylbenzene)-N′-ethylsulfonamido}-phenyl]-N-[2-(N″-methylpiperidin-2-yl)ethyl]propionamide (96);

3-[2′-{(4″-chloro-2″,5″-dimethylbenzene)-N′-ethylsulfonamido}-phenyl]-N-[2-(N″-methylpiperidin-3-yl)ethyl]propionamide (97);

3-[2′-(benzene-N′-methylsulfonamido}-phenyl]-N-[2-(N″,N″-dimethylpiperidin-4-yl)ethyl]propionamide (98);

3-[2′-{(2″,3″-dichlorobenzene)-N′-methylsulfonamido}-phenyl]-N-[2-(N″,N″-dimethylpiperidin-4yl)ethyl]propionamide (99);

3-[2′-{(2″-chlorobenzene)-N′-methylsulfonamido}-phenyl]-N-[2-(N″,N″-dimethylpiperidin-4-yl)ethyl]propionamide (100);

3-[2′-{(2″-methylbenzene)-N′-methylsulfonamido}-phenyl]-N-[2-(N″, N″-dimethylpiperidin-4-yl)ethyl]propionamide (101);

3-[2′-{(3″-chlorobenzene)-N′-methylsulfonamido}-phenyl]-N-[2-(N″,N″-dimethylpiperidin-4-yl)ethyl]propionamide (102);

3-[2′-{(3″-methylbenzene)-N′-methylsulfonamido}-phenyl]-N-[2-(N″,N″-dimethylpiperidin-4-yl)ethyl]propionamide (103);

3-[2′-{(2″,4″,5″-trichlorobenzene)-N′-methylsulfonamido}-phenyl]-N-[2-(N″,N″-dimethylpiperidin-4-yl)ethyl]propionamide (104);

3-[2′-{(2″,4″-dichloro-5″-methylbenzene)-N′-methylsulfonamido}-phenyl]-N-[2-(N″,N″-dimethylpiperidin-4-yl)ethyl]propionamide (105);

3-[2′-{(2″,5″-dimethylbenzene)-N′-methylsulfonamido}-phenyl]-N-[2-(N″,N″-dimethylpiperidin-4-yl)ethyl]propionamide (106);

3-[2′-{(2″,5″-dichlorobenzene)-N′-methylsulfonamido}-phenyl]-N-[2-(N″,N″-dimethylpiperidin-4-yl)ethyl]propionamide (107);

3-[2′-{(2″,6″-dichlorobenzene)-N′-methylsulfonamido}-phenyl]-N-[2-(N″,N″-dimethylpiperidin-4-yl)ethyl]propionamide (108);

3-[2′-{(2″-methoxy-5″-chlorobenzene)-N′-methylsulfonamido}-phenyl]-N-[2-(N″,N″-dimethylpiperidin-4-yl)ethyl]propionamide (109);

3-[2′-{(2″-methyl-5″-fluorobenzene)-N′-methylsulfonamido}-phenyl]-N-[2-(N″,N″-dimethylpiperidin-4-yl)ethyl]propionamide (110);

3-[2′-{(2″-chloro-6″-methylbenzene)-N″-methylsulfonamido}-phenyl]-N-[2-(N″,N″-dimethylpiperidin-4-yl)ethyl]propionamide (111);

3-[2′-{(3″-fluoro-4″-methylbenzene)-N′-methylsulfonamido}-phenyl]-N-[2-(N″,N″-dimethylpiperidin-4-yl)ethyl]propionamide (112);

3-[2′-{(naphthal-2-yl)-N′-methylsulfonamido}-phenyl]-N-[2-(N″,N″-dimethylpiperdin-4-yl)ethyl]propionamide (113);

3-[2′-{(3″,4″-difluorobenzene)-N′-methylsulfonamido}-phenyl]-N-[2-(N″,N″-dimethylpiperidin-4-yl)ethyl]propionamide (114);

3-[2′-{(3″-chloro-4″-fluorobenzene)-N′-methylsulfonamido}-phenyl]-N-[2-(N″,N″dimethylpiperidin-4-yl)ethyl]propionamide (115);

3-[2′-{(3″,4″-dimethoxybenzene)-N′-methylsulfonamido}-phenyl]-N-[2-(N″,N″-dimethylpiperidin-4-yl)ethyl]propionamide (116);

3-[2′-{(2″-chloro-4″-cyanobenzene)-N′-methylsulfonamido}-phenyl]-N-[2-(N″,N″-dimethylpiperidin-4-yl)ethyl]propionamide (117);

3-[2′-{(2″,4″-dichloro-5″-methylbenzene)-N′-methylsulfonamido}-phenyl]-N-[2-(N″,N″-dimethylpiperidin-4-yl)ethyl]propionamide (118);

3-[2′-{(4″-chloro-2″,5″-dimethylbenzene)-N′-methylsulfonamido}-phenyl]-N-methyl-N-[2-{N″-(4-methylpyrid-2-yl)}piperidin-4-yl]ethyl]propionamide (119);

3-[2′-{(4-chloro-2″,5″-dimethylbenzene)-N′-methylsulfonamido}-phenyl]-N-[2-(N″,N″-dimethylpiperidin-4-yl)ethyl]propionamide (120);

3-[2′-{(naphthalyl)-N′-methylsulfonamido}-phenyl]-N-[2-(N″,N″-dimethylpiperidin-4-yl)ethyl]propionamide (121);

3-[2′-{(4-chlorobenzo[c][1,2,5]oxadiazol-7-yl)-N′-methylsulfonamido}-phenyl]-N-[2-(N″,N″-dimethylpiperidin-4-yl)ethyl]propionamide (122);

3-[2′-{(2″-phenoxybenzene)-N′-methylsulfonamido}-phenyl]-N-[2-(N″,N″-dimethylpiperidin-4-yl)ethyl]propionamide (123);

3-[2′-{(2″,3″,4″-trifluorobenzene)-N′-methylsulfonamido}-phenyl]-N-[2-(N″,N″-dimethylpiperidin-4-yl)ethyl]propionamide (124);

3-[2′-{(2″-chloro-4″-trifluoromethylbenzene)-N′-methylsulfonamido}-phenyl]-N-[2-(N″,N″-dimethylpiperidin-4-yl)ethyl]propionamide (125);

3-[2′-{(2″-methyl-4″-fluorobenzene)-N′-methylsulfonamido}-phenyl]-N-[2-(N″,N″-dimethylpiperidin-4-yl)ethyl]propionamide (126);

3-2′-{(3″,5″-dichloropryid-2-yl)-N′-methylsulfonamido}-phenyl]-N-[2-(N″,N″-dimethylpiperidin-4-yl)ethyl]propionamide (127);

3-[2′-{(2″,3″,4″-trichlorobenzene)-N′-methylsulfonamido}-phenyl]-N-[2-(N″,N″-dimethylpiperidin-4-yl)ethyl]propionamide (128);

3-[2′-{(3″,5″-dichlorobenzene)-N′-methylsulfonamido}-phenyl]-N-[2-(N″,N″-dimethylpiperidin-4-yl)ethyl]propionamide (129);

3-[2′-{(4″-chloro-2″,5″-dimethylbenzene)-N′-ethylsulfonamido}phenyl]-N-methyl-N-[2-[N″-(pyrimidin-4-yl)piperidin-4-yl]ethyl]propionamide (130);

3-(2′-{(4″-chloro-2″,5″-dimethylbenzene)-N′-ethylsulfonamido}phenyl]-N-methyl-N-[2-[N″-(5-methylpyrimidin-4-yl)piperidin-4-yl]ethyl]propionamide (131);

3-[2′-{(4″-chloro-2″,5″-dimethylbenzene)-N′-methylsufonamido}phenyl]-N-methyl-N-[2-[4-(piperidinylmethyl)phenyl]ethyl]propionamide (132);

3-[2′-{(4″-chloro-2″,5″-dimethylbenzene)-N′-methylsulfonamido}phenyl]-N-methyl-N-{2-[3H-pyrrolo[3,2-b]pyrid-2-yl]ethyl}propionamide (133); and

3-[2′-{(2″,6″-dichlorobenzene)-N′-ethylsulfonamido}-phenyl]-N-[2-(N″-methylpiperidin-4-yl)ethyl]propionamide (134)

and pharmaceutically acceptable salts thereof.

TABLE II

| | |
|---|---|
| | III |
| 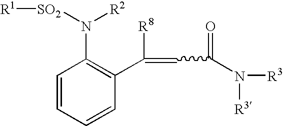 | |

(R^{3′} and R⁸ are hydrogen unless otherwise noted)

| Ex # | R¹ | R⁸ | R² | R³ |
|---|---|---|---|---|
| 22 | 2,5-dimethyl-4-chlorophenyl | | —CH₃ | 1-(R)-(N-pyrrolidinylcarbon-yl)-2-(4-py- |
| | | | | ridyl)ethyl |
| 23 | 2,5-dimethyl-4-chlorophenyl | | —CH₃ | 2-(N-methylpiperidin-4-yl)ethyl |
| 24 | 2,5-dimethyl-4-chlorophenyl | | —CH₃ | 1-(R)-1-(N-piperidinylcar-bonyl)ethyl |
| 25 | 2,5-dimethyl-4-chlorophenyl | | —CH₃ | 1-(S)-1-(N-piperidinylcar-bonyl)ethyl |
| 26 | 2,5-dimethyl-4-chlorophenyl | | —CH₃ | 2-[N-(4-methylpyrid-2-yl)piperidin-4-yl]ethyl |
| 27 | 2,5-dimethyl-4-chlorophenyl | | —CH₃ | 1-(R)-(N-pyrrolidinylcarbon-yl)-2-[(4-py- |
| | | | | ridyl)phen-4-yl]ethyl |
| 28 | 2,5-dimethyl-4-chlorophenyl | | —CH₃ | 2-(N-piperidinyl)ethyl |
| 29 | 2,5-dimethyl-4-chlorophenyl | | —CH₃ | 2-(4-pyridyl)ethyl |
| 30 | 2,5-dimethyl-4-chlorophenyl | | —CH₃ | 2-[N-(2-pyridyl)piperidin-4-yl]ethyl |
| 31 | 2,5-dimethyl-4-chlorophenyl | | —CH₃ | 2-[N-(ethyl)piperidin-4-yl]ethyl |
| 32 | 2,5-dimethyl-4-chlorophenyl | | —CH₃ | 1-(S)-1-methyl-2-(N-piperidinyl)ethyl |
| 33 | 2,5-dimethyl-4-chlorophenyl | | —CH₃ | 2-{N″-(pyrid-4-yl)piperidin-4-yl}ethyl |
| 34 | 2,5-dimethyl-4-chlorophenyl | | —CH₃ | 1-(R)-1-methyl-2-(N-piperidinyl)ethyl |
| 35 | 2,5-dimethyl-4-chlorophenyl | | —CH₃ | 2-[N-(3-methylpyrid-2-yl)piperidin-4-yl]ethyl |
| 36 | 2,5-dimethyl-4-chlorophenyl | | —CH₃ | 1-(S)-1-methyl-2-(4-methylpiperazin-1-yl)ethyl |
| 37 | 2,5-dimethyl-4-chlorophenyl | | —CH₃ | 1-(R)-1-methyl-2-(4-methylpiperazin-1-yl)ethyl |
| 38 | 2,5-dimethyl-4-chlorophenyl | | —CH₃ | R^{3′} = —CH₃ |
| | | | | R³ = 2-(N-methyl-piperidin-4-yl)ethyl |
| 39 | 2,3-dichloro-phenyl | | —CH₃ | R^{3′} = —CH₃ |
| | | | | R³ = 2-(N-methyl-piperidin-4-yl)ethyl |
| 40 | 2,5-dimethyl-4-chlorophenyl | | —CH₃ | —(R,S)-methoxycarbonylbenzyl |
| 41 | 2,3-dichloro-phenyl | | —CH₃ | —(R,S)-methoxycarbonylbenzyl |
| 42 | 2,5-dimethyl-4-chlorophenyl | | —C₂H₅ | 2-N-ethylpiperidin-4-yl)ethyl |
| 135 | 2,6-dichlorophenyl | | CH₂CH₃ | 2-(N-methylpiperidin-4-yl)ethyl |
| 136 | 2,6-dichlorophenyl | | CH₂CH₃ | 4-[1-(pyrid-4-yl)-piperazin-4-yl]phenyl |
| 137 | 2,6-dichlorophenyl | | CH₂CH₃ | 2-(N-(pyrid-4-yl)-piperidin-4-yl)ethyl |
| 138 | 2,5-dimethyl-4-chlorophenyl | CF₃ | CH₃ | 2-(N-(pyrid-4-yl)-piperidin-4-yl)ethyl |

Particularly preferred compounds include the following compounds and pharmaceutically acceptable salts thereof:

3-[2′-{(4″-chloro-2″,5″-dimethylbenzene)-N′-methylsulfonamido}-phenyl]-N-[1-(R)-1-(N″-pyrrolidinylcarbonyl)-2-(4-pyridyl)eth-1-yl]acrylamide (22);

3-[2′-{(4″-chloro-2″,5″-dimethylbenzene)-N′-methylsulfonamido}-phenyl]-N-[2-(N″-methylpiperidin-4-yl)eth-1-yl]acrylamide (23);

3-[2′-{(4″-chloro-2″,5″-dimethylbenzene)-N′-methylsulfonamido}-phenyl]-N-[1-(R)-1-(N″-piperidinylcarbonyl]eth-1-yl]acrylamide (24);

3-[2′-{(4″-chloro-2″,5″-dimethylbenzene)-N′-methylsulfonamido}-phenyl]-N-[1-(S)-1-(N″-piperidinylcarbony]eth-1-yl]acrylamide (25);

3-[2′-{(4″-chloro-2″,5″-dimethylbenzene)-N′-methylsulfonamido}-phenyl]-N-[2-{N″-(4-methylpyrid-2-yl)}piperidin-4-yl]eth-1-yl]acryl-mide (26);

3-[2′-{(4″-chloro-2″,5″-dimethylbenzene)-N′-methylsulfonamido}-phenyl]-N-[1-(R)-1-(N″-pyrrolidinylcarbonyl)-2-(4-pyridylphen-4-yl)eth-1-yl]acrylamide (27);

3-[2′-{(4″-chloro-2″,5″-dimethylbenzene)-N′-methylsulfonamido}-phenyl]-N-[(2-N″-piperidinyl)eth-1-yl]acrylamide (28);

3-[2′-{(4″-chloro-2″,5″-dimethylbenzene)-N′-methylsulfonamido}-phenyl]-N-[(2-pyrid-4-yl)eth-1-yl]acrylamide (29);

3-[2′-{(4″-chloro-2″,5″-dimethylbenzene)-N′-methylsulfonamido}-phenyl]-N-[2-{N″-{(2-pyridyl)piperidin-4-yl}eth-1-yl]acrylamide (30);

3-[2′-{(4″-chloro-2″,5″-dimethylbenzene)-N′-methylsulfonamido}-phenyl]-N-(2-(N″-ethylpiperidin-4-yl)eth-1-yl]acrylamide (31);

3-[2′-{(4″-chloro-2″,5″-dimethylbenzene)-N′-methylsulfonamido}-phenyl]-N-[1-(S)-1-methyl-2-N″-piperidinyl)eth-1-yl]acrylamide (32);

3-[2′-{(4″-chloro-2″,5″-dimethylbenzene)-N′-methylsulfonamido}-phenyl]-N-[2-{N″-(pyrid-4-yl}piperidin-4-yl)eth-1-yl]acrylamide (33);

3-[2′-{(4″-chloro-2″,5″-dimethylbenzene)-N′-methylsulfonamido}-phenyl]-N-[1-(R)-1-methyl-2-(N″-piperidinyl)eth-1-yl]acrylamide (34);

3-[2′-{(4″-chloro-2″,5″-dimethylbenzene)-N′-methylsulfonamido}-phenyl]-N-[2-{N″-(3-methylpyrid-2-yl}piperidin-4-yl)eth-1-yl]acrylamide (35);

3-[2′-{(4″-chloro-2″,5″-dimethylbenzene)-N′-methylsulfonamido}-phenyl]-N-[1-(S)-1-methyl-2-(4-methylpiperazin-1-yl)eth-1-yl]acryl-amide (36);

3-[2′-{(4″-chloro-2″,5″-dimethylbenzene)-N′-methylsulfonamido}-phenyl]-N-[1-(R)-1-methyl-2-(4-methylpiperazin-1-yl)eth-1-yl]acryl-amide (37);

3-[2′-{(4″-chloro-2″,5″-dimethylbenzene)-N′-methylsulfonamido}-phenyl]-N-methyl-N-2-(N″-methylpiperidin-4-yl)eth-1-yl]acrylamide (38);

3-[2′-{(2″,3″-dichlorobenzene)-N′-methylsulfonamido}phenyl]-N-methyl-N-2-[(N″-methylpiperidin-4-yl)eth-1-yl]acrylamide (39);

3-[2′-{(2″,5″-dimethyl-4″-chlorobenzene)-N′-methylsulfonamido}-phenyl]-N-[(α-(R,S)-methoxycarbonyl)benzyl]acrylamide (40);

3-[2′-{(2″,3″-dichlorobenzene)-N′-methylsulfonamido}phenyl]-N-N-[(α-(R,S)-methoxycarbonyl)benzyl]acrylamide (41);

3-[2′-{(4″-chloro-2″,5″-dimethylbenzene)-N′-ethylsulfonamido}-phenyl]-N-[2-(N″-ethylpiperidin-4-yl)eth-1-yl]acrylamide (42);

3-[2′-{(2″,6″-dichlorobenzene)-N′-ethylsulfonamido}-phenyl]-N-[2-(N″-methylpiperidin-4-yl)ethyl]acrylamide (135);

3-[2′-{(2″,6″-dichlorobenzene)-N′-ethylsulfonamido}-phenyl]-N-[4-N″-(pyrid-4-yl)piperazinyl)phenyl]acrylamide (136);

3-[2′-{(2″,6″-dichlorobenzene)-N′-ethylsulfonamido}-phenyl]-N-[2-{N″-(pyrid-4-yl}piperidin-4-yl)eth-1-yl]acrylamide (137); and

3-trifluoromethyl-3-[2′-{(4″-chloro-2″,5″-dimethylbenzene)-N′-methylsulfonamido}-phenyl]-N-[2-{N″-(pyrid-4-yl}piperidin-4-yl)eth-1-yl]acrylamide (138).

and pharmaceutically acceptable salts thereof.

TABLE III

| | |
|---|---|
| | IV |
| 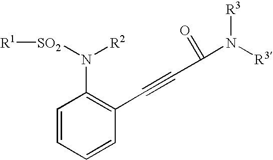 | |

(R^{3′} is hydrogen unless otherwise noted)

| Ex # | R¹ | R² | R³ |
|---|---|---|---|
| 43 | 2,5-dimethyl-4-chlorophenyl | —CH₃ | 1-(R)-(N-pyrrolidinylcarbon-yl)-2-(4-py- |
| | | | ridyl)ethyl |
| 44 | 2,5-dimethyl-4-chlorophenyl | —CH₃ | 2-(N-methylpiperidin-4-yl)ethyl |
| 45 | 2,5-dimethyl-4-chlorophenyl | —CH₃ | 1-(R)-1-(N-piperidinylcar-bonyl)ethyl |
| 46 | 2,5-dimethyl-4-chlorophenyl | —CH₃ | 1-(S)-1-(N-piperidinylcar-bonyl)ethyl |
| 47 | 2,5-dimethyl-4-chlorophenyl | —CH₃ | 2-[N-(4-methylpyrid-2-yl)piperidin-4-yl]ethyl |
| 48 | 2,5-dimethyl-4-chlorophenyl | —CH₃ | 1-(R)-(N-pyrrolidinylcarbon-yl)-2-[(4-py- |
| | | | ridyl)phen-4-yl]ethyl |
| 49 | 2,5-dimethyl-4-chlorophenyl | —CH₃ | 2-(N-piperidinyl)ethyl |
| 50 | 2,5-dimethyl-4-chlorophenyl | —CH₃ | 2-(4-pyridyl)ethyl |
| 51 | 2,5-dimethyl-4-chlorophenyl | —CH₃ | 2-[N-(2-pyridyl)piperidin-4-yl]ethyl |
| 52 | 2,5-dimethyl-4-chlorophenyl | —CH₃ | 2-[N-(ethyl)piperidin-4-yl]ethyl |
| 53 | 2,5-dimethyl-4-chlorophenyl | —CH₃ | 1-(S)-1-methyl-2-(N-piperidinyl)ethyl |
| 54 | 2,5-dimethyl-4-chlorophenyl | —CH₃ | 2-{N″-(pyrid-4-yl)piperidin-4-yl}ethyl |
| 55 | 2,5-dimethyl-4-chlorophenyl | —CH₃ | 1-(R)-1-methyl-2-(N-piperidinyl)ethyl |
| 56 | 2,5-dimethyl-4-chlorophenyl | —CH₃ | 2-[N-(3-methylpyrid-2-yl)piperidi-4-yl]ethyl |
| 57 | 2,5-dimethyl-4-chlorophenyl | —CH₃ | 1-(S)-1-methyl-2-(4-methylpiperazin-1-yl)ethyl |
| 58 | 2,5-dimethyl-4-chlorophenyl | —CH₃ | 1-(R)-1-methyl-2-(4-methylpiperazin-1-yl)ethyl |
| 59 | 2,5-dimethyl-4-chlorophenyl | —CH₃ | R^{3′} = —CH₃ |
| | | | R³ = 2-(N-methyl-piperidin-4-yl)ethyl |
| 60 | 2,3-dichloro-phenyl | —CH₃ | R^{3′} = —CH₃ |
| | | | R³ = 2-(N-methyl-piperidin-4-yl)ethyl |
| 61 | 2,5-dimethyl-4-chlorophenyl | —CH₃ | —(R,S)-methoxycarbonylbenzyl |
| 62 | 2,3-dichloro-phenyl | —CH₃ | —(R,S)-methoxycarbonylbenzyl |
| 63 | 2,5-dimethyl-4-chlorophenyl | —C₂H₅ | 2-(N-ethylpiperidin-4-yl)ethyl |

Particularly preferred compounds include the following compounds and pharmaceutically acceptable salts thereof:

3-[2′-{(4″-chloro-2″,5″-dimethylbenzene)-N′-methylsulfonamido}-phenyl]-N-[1-(R)-1-(N″-pyrrolidinylcarbonyl)-2-(4-pyridyl)eth-1-yl]propargylamide (43);

3-[2′-{(4″-chloro-2″,5″-dimethylbenzene)-N′-methylsulfonamido}-phenyl]-N-[2-(N″-methylpiperidin-4-yl)eth-1-yl]propargylamide (44);

3-[2′-{(4″-chloro-2″,5″-dimethylbenzene)-N′-methylsulfonamido}-phenyl]-N-[1-(R)-1-(N″-piperidinylcarbonyl]eth-1-yl]propargylamide (45);

3-[2′-{(4″-chloro-2″,5″-dimethylbenzene)-N′-methylsulfonamido}-phenyl]-N-[1-(S)-1-(N″-piperidinylcarbonyl]eth-1-yl]propargylamide (46);

3-[2′-{(4″-chloro-2″,5″-dimethylbenzene)-N′-methylsulfonamido}-phenyl]-N-[2-{N″-(4-methylpyrid-2-yl)}piperidin-4-yl]eth-1-yl]propargylamide (47);

3-[2′-{(4″-chloro-2″,5″-dimethylbenzene)-N′-methylsulfonamido}-phenyl]-N-[1-(N″-pyrrolidinylcarbonyl)-2-(4-pyridylphen-4-yl)eth-1-yl]propargylamide (48);

3-[2′-{(4″-chloro-2″,5″-dimethylbenzene)-N′-methylsulfonamido}-phenyl]-N-[(2-N″-piperidinyl)eth-1-yl]propargylamide (49);

3-[2′-{(4″-chloro-2″,5″-dimethylbenzene)-N′-methylsulfonamido}-phenyl]-N-[(2-pyrid-4-yl)eth-1-yl]propargylamide (50);

3-[2′-{(4″-chloro-2″,5″-dimethylbenzene)-N′-methylsulfonamido}-phenyl]-N-[2-{N″-{(2-pyridyl)piperidin-4-yl}eth-1-yl]propargylamide (51);

3-[2′-{(4″-chloro-2″,5″-dimethylbenzene)-N′-methylsulfonamido}-phenyl]-N-[2-(N″-ethylpiperidin-4-yl)eth-1-yl]propargylamide (52);

3-[2′-{(4″-chloro-2″,5″-dimethylbenzene)-N′-methylsulfonamido}-phenyl]-N-[1-(S)-1-methyl-2-(N″-piperidinyl)eth-1-yl]propargylamide (53);

3-[2′-{(4″-chloro-2″,5″-dimethylbenzene)-N′-methylsulfonamido}-phenyl]-N-[2-{N″-(pyrid-4-yl}piperidin-4-yl)eth-1-yl]propargylamide (54);

3-[2′-{(4″-chloro-2″,5″-dimethylbenzene)-N′-methylsulfonamido}-phenyl]-N-[1-(R)-1-methyl-2-(N″-piperidinyl)eth-1-yl]propargylamide (55);

3-[2′-{(4″-chloro-2″,5″-dimethylbenzene)-N′-methylsulfonamido}-phenyl]-N-[2-{N″-3-methylpyrid-2-yl}piperidin-4-yl)eth-1-yl]pro-pargylamide (56);

3-[2′-{(4″-chloro-2″,5″-dimethylbenzene)-N′-methylsulfonamido}-phenyl]-N-[1-(S)-1-methyl-2-(4-methylpiperazin-1-yl)eth-1-yl]propargylamide (57);

3-[2′-{(4″-chloro-2″,5″-dimethylbenzene)-N′-methylsulfonamido}-phenyl]-N-[1-(R)-1-methyl-2-(4-methylpiperazin-1-yl)eth-1-yl]propargylamide (58);

3-[2′-{(4″-chloro-2″,5″-dimethylbenzene)-N′-methylsulfonamido}-phenyl]-N-methyl-N-2-(N″-methylpiperidin-4-yl)eth-1-yl]propargylamide (59);

3-[2′-{(2″,3″-dichlorobenzene)-N′-methylsulfonamido}phenyl]-N-methyl-N-2-[(N″-methylpiperidin-4-yl)eth-1-yl]propargylamide (60);

3-[2′-{(2″,5″-dimethyl-4″-chlorobenzene)-N′-methylsulfonamido}-phenyl]-N-[(α-(R,S)-methoxycarbonyl)benzyl]propargylamide (61);

3-[2′-{(2″,3″-dichlorobenzene)-N′-methylsulfonamido}phenyl]-N-N-[(α-(R,S)-methoxycarbonyl)benzyl]propargylamide (62); and

3-[2′-{(4″-chloro-2″,5″-dimethylbenzene)-N′-ethylsulfonamido}-phenyl]-N-[2-(N″-ethylpiperidin-4-yl)eth-1-yl]propargylamide (63).

TABLE IV

| | |
|---|---|
| | V |
| 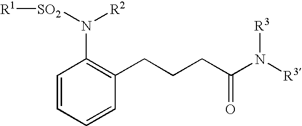 | |

(R^{3′} is hydrogen unless otherwise noted)

| Ex # | R¹ | R² | R³ |
|---|---|---|---|
| 64 | 2,3-dichloro-phenyl | —CH₃ | —(R,S)-methoxycarbonylbenzyl |
| 65 | 2,5-dimethyl-4-chloro- | —CH₃ | —(R,S)-methoxycarbonylbenzyl |
| | phenyl | | |
| 139 | 2,5-dimethyl-4-chloro- | CH₃ | 2-(N-methylpiperidin-4-yl)ethyl |
| | phenyl | | R^{3′} = methyl |

Particularly preferred compounds include the following compounds and pharmaceutically acceptable salts thereof:

3-[2′-{(2″,5″-dimethyl-4-chlorobenzene)-N′-methylsulfonamido}phenyl]-N-[(α-(R,S)-methoxycarbonyl)benzyl]butyramide (64); and

3-[2′-{(2″,3″-dichlorobenzene)-N′-methylsulfonamido}phenyl]-N-N-[(α-(R,S)-methoxycarbonyl)benzyl]butyramide (65); and

3-[2′-{(2″,5″-dimethyl-4″-chlorobenzene)-N′-methylsulfonamido}phenyl-N-methyl-N-[2-N″-methylpiperidin-4-yl)ethyl]butyramide (139);

and pharmaceutically acceptable salts thereof.

Further, references to the compounds of Formula I-V with respect to pharmaceutical applications thereof are also intended to include pharmaceutically acceptable salts of the compounds of these formulas.

The invention also provides methods for determining bradykinin levels in a biological sample which comprises contacting said biological sample with a compound of Formula I-V, at a predetermined concentration and then measuring the level of binding. Such measurements are well within the skill of the art using well known techniques such as ELISA assays and the like.

The present invention also provides a pharmaceutical composition comprising a pharmaceutically acceptable carrier and a therapeutically amount of a compound of Formula I-V or mixtures thereof effective to treat or palliate adverse symptoms associated with the presence of bradykinin in mammals.

The present invention further provides a method for treating or palliating adverse symptoms mediated at least in part by the presence or secretion of bradykinin in mammals which comprises administering a therapeutically effective amount of a compound Formula I-V or mixtures thereof or as is more generally the case the pharmaceutical composition.

The present invention provides a method for treating or ameliorating pain, hyperalgesia, hyperthermia and/or edema in mammals mediated at least in part by the release of bradykinin in such mammals which comprises a therapeutically effective amount of a compound Formula I-V or mixtures thereof or as is more generally the case the pharmaceutical composition.

The present invention provides a method for treating or ameliorating adverse symptoms mediated at least in part by the release of bradykinin relative to burns, perioperative pain, migraine, shock, central nervous system injury, asthma, rhinitis, premature labor, inflammatory arthritis, inflammatory bowel disease or neuropathic pain.

### DETAILED DESCRIPTION OF THE PREFERRED EMBODIMENTS

As noted above, this invention is directed to certain substituted N-phenyl sulfonamide derivatives and related compounds which are useful as bradykinin antagonists to relieve adverse symptoms in mammals mediated, at least in part, by bradykinin. However, prior to describing this invention in further detail, the following terms will first be defined.

Definitions

Unless otherwise expressly defined with respect to a specific occurrence of the term, the following terms as used herein shall have the following meanings regardless of whether capitalized or not:

First, for the sake of clarity, the following nomenclature system was employed in this application:

1. For compounds of formula I where Q is ethylene, these were named based on the core propionamide structure as follows:

with the conventional numbering system employed.

2. The phenyl substituent at the 3-position of the propionamide structure is shown and uses primes to distinguish over the numbering of the propionamide structure:

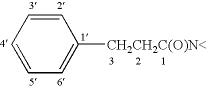

3. The substituted phenyl-N-methylsulfonamide structure found at the 2′ position provides the following structure wherein double primes are employed on the substituted phenyl of the sulfonylamide to distinguish over other numbering systems employed:

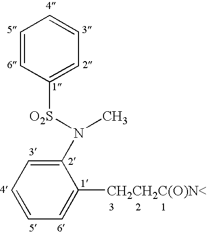

4. The substitution pattern on the amide of the propionamide employs triple primes to distinguish over the other numbering systems employed. For example, the following compound has the following number system on the substituent of the amide of the propionamide:

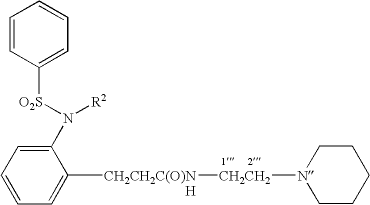

5. Nitrogen substitution off of the amino group of the propionamide is referred to as N-substituted where the substituent group is recited;

nitrogen substitution off of the amino group of the sulfonamide is referred to as N′-substituted where the substituent group is recited; and

nitrogen substitution off of an amino group of the substituent off of the propionamide is referred by N″—as shown above.

Compounds of formula I where Q is ethenylene were named similarly to the propionamide structures above but using an acrylamide core structure as shown:

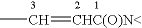

Compounds of formula I where Q is ethynylene were named similarly to the propionamide and acrylamide structures above but using a propargylamide core structure as shown:

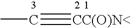

Other compounds within the scope of this invention could similarly be named.

The term “alkyl” refers to an alkyl group, of from 1 to 10 carbon atoms, more preferably, 1 to 6 carbon atoms which is exemplified by the groups methyl, ethyl, n-propyl, iso-propyl, n-butyl, t-butyl, n-hexyl, n-decyl, and the like.

The term “substituted allyl” refers to an alkyl group, of from 1 to 10 carbon atoms, more preferably, 1 to 6 carbon atoms, having from 1 to 5 substituents, preferably 1 to 3 substituents, independently selected from the group consisting of alkoxy, substituted alkoxy, acyl, acylamino, amino, substituted amino, aminoacyl, aryl, substituted aryl, aryloxy, substituted aryloxy, cyano, halogen, hydroxyl, nitro, oxo, thioxo, carboxyl, carboxylalkyl, carboxyl substituted alkyl, carboxylaryl, carboxyl substituted aryl, carboxylheteroaryl, carboxyl substituted heteroaryl, carboxylheterocyclic, carboxyl substituted heterocyclic, cycloalkyl, substituted cycloalkyl, heteroaryl, substituted heteroaryl, heterocyclic and substituted heterocyclic.

“Alkylene” refers to divalent hydrocarbon radicals of 2-3 carbon atoms which can be branched or unbranched, optionally substituted with 1 to 2 substituents selected from halo, alkyl of 1 to 3 carbon atoms (optionally substituted with from 1 to 7 halo groups, for example trifluoromethyl), benzyl, or phenyl. Examples include ethylene (—CH₂CH₂—), 1-methylethylene (—CH(CH₃)CH₂—), 2-methylethylene (—CH₂CH(CH₃)—), n-propylene (—CH₂CH₂CH₂—), and 1-trifluoromethylethylene (—CH(CF₃)CH₂—).

“Alkenyl” refers to alkenyl groups having from 2 to 10 carbon atoms and more preferably 2 to 6 carbon atoms and having at least 1 and preferably from 1-2 sites of alkenyl unmaturation.

“Substituted alkenyl” refers to alkenyl groups having from 1 to 5 substituents, preferably 1 to 3 substituents, independently selected from the group of substituents defined for substituted alkyl.

“Alkenylene” refers to divalent vinyl unsaturated hydrocarbon radicals of 2-3 carbon atoms which can be branched or uabranched, optionally substituted with 1 to 2 substituents selected from halo or alkyl of 1 to 3 carbon atoms (optionally substituted with from 1 to 7 halo groups, for example trifluoromethyl). Examples include ethenylene (—CH═CH—), 1-methylethenylene (—C(CH₃)═CH—), 2-methylethenylene (—CH═C(CH₃)—), 1-propenylene (—CH═CHCH₂—) and 2-propenylene (—CH₂CH═CH—) including both cis and trans isomers.

“Alkynyl” refers to alkynyl groups having from 2 to 10 carbon atoms and more preferably 3 to 6 carbon atoms and having at least 1 and preferably from 1-2 sites of alkynyl unsaturation.

“Substituted alkynyl” refers to alkynyl groups having from 1 to 5, preferably 1 to 3 substituents, selected from the same group of substituents as defined for substituted alkyl.

“Alkynylene” refers to divalent acetylenic unsaturated hydrocarbon radicals of 2-3 carbon atoms which includes ethynylene (—C≡C—), 1-propynylene (—C≡CCH₂—) and 2-propynylene (—CH₂C≡C—).

“Alkoxy” refers to the group “alkyl-O—” which includes, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Substituted alkoxy” refers to the group “substituted alkyl-O—”.

“Acyl” refers to the groups H—C(O)—, alkyl-C(O)—, substituted alkyl-C(O)—, cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)— provided that a nitrogen atom of the heterocyclic or substituted heterocyclic is not bound to the —C(O)— group wherein alkyl, substituted alkyl, cycloalkyl, substituted cycloalkyl, aryl, substituted aryl, heteroaryl, substituted heteroaryl, heterocyclic and substituted heterocyclic are as defined herein.

“Amino” refers to the group —NH₂.

“Substituted amino” refers to the group —NRR, where each R group is independently selected from the group consisting of hydrogen, alkyl, substituted alkyl, cycloalkyl, substituted cycloalkyl, aryl, substituted aryl, heteroaryl, substituted heteroaryl, heterocyclic, and substituted heterocyclic or, optionally, each R is joined together with the nitrogen atom bound thereto to form a heterocyclic or substituted heterocyclic group.

The “acylamino” or as a prefix “carbamoyl” or “carboxamide” or “substituted carbamoyl” or “substituted carboxamide” refers to the group —C(O)NRR where each R is independently selected from the group consisting of hydrogen, alkyl, substituted alkyl, aryl, substituted aryl, cycloalkyl, substituted cycloalkyl, heteroaryl, substituted heteroaryl, heterocyclic, substituted heterocyclic and where each R is joined to form together with the nitrogen atom a heterocyclic or substituted heterocyclic wherein alkyl, substituted alkyl, cycloalkyl, substituted cycloalkyl, aryl, substituted aryl, heteroaryl, substituted heteroaryl, heterocyclic and substituted heterocyclic are as defined herein.

“Aminoacyl” refers to the groups —NR′C(O)alkyl, —NR′C(O)substituted alkyl, —NR′C(O)cycloalkyl, —NR′C(O)substituted cycloalkyl, —NR′C(O)aryl, —NR′C(O)substituted aryl, —NR′C(O)heteroaryl, —NR′C(O)substituted heteroaryl, —NR′C(O)heterocyclic, and —NR′C(O)substituted heterocyclic where R′ is hydrogen or alkyl and wherein alkyl, substituted alkyl, cycloalkyl, substituted cycloalkyl, aryl, substituted aryl, heteroaryl, substituted heteroaryl, heterocyclic and substituted heterocyclic are defined herein.

“Aryl” or “Ar” refers to an aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like). Preferred aryls include phenyl and naphthyl.

“Substituted aryl” refers to aryl groups which are substituted with from 1 to 5, preferably 1-3, substituents selected from the group consisting of hydroxy, acyl, acylamino, alkyl, substituted alkyl, alkoxy, substituted alkoxy, amino, substituted amino, aminoacyl, aryl, substituted aryl, aryloxy, substituted aryloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cyano, cycloalkyl, substituted cycloalkyl, halo, nitro, heteroaryl, substituted heteroaryl, heterocyclic and substituted heterocyclic, wherein each of the terms is as defined herein.

“Aryloxy” refers to the group —O-aryl where aryl is as defined herein.

“Substituted aryloxy” refers to the group —O-substituted aryl where substituted aryl is as defined herein.

“Aralkyl” refers to the group -alkyl-aryl where alkyl and aryl are as defined herein. Such groups are exemplified, for example, by benzyl and phenethyl.

“Carboxyl” refers to the group —COOH and pharmaceutically acceptable salts thereof.

“Carboxylalkyl” refers to the group —COO-alkyl where alkyl is as defined herein.

“Carboxyl-substituted alkyl” refers to the group —COO-substituted alkyl here substituted alkyl is as defined herein.

“Carboxyl-cycloalkyl” refers to the group —COO-cycloalkyl where cycloalkyl is as defined herein.

“Carboxyl-substituted cycloalkyl” refers to the group —COO-substituted cycloalkyl where substituted cycloalkyl is as defined herein.

“Carboxylaryl” refer to the group —COO-aryl where aryl is as defined herein.

“Carboxyl-substituted aryl” refer to the group —COO-substituted aryl where substituted aryl is as defined herein.

“Carboxylheteroaryl” refer to the group —COO-heteroaryl where heteroaryl is as defined herein.

“Carboxyl-substituted heteroaryl” refer to the group —COO-substituted heteroaryl where substituted heteroaryl is as defined herein.

“Carboxylheterocyclic” refer to the group —COO-heterocyclic where heterocyclic is as defined herein.

“Carboxyl-substituted heterocyclic” refer to the group —COO-substituted heterocyclic where substituted heterocyclic is as defined herein.

“Cycloalkyl” refers to cyclic alkyl groups of from 3 to 10 carbon atoms having a single or multiple cyclic rings including, by way of example, cyclopropyl, cyclobutyl, cyclopentyl, cyclooctyl, adamantanyl, and the like.

“Substituted cycloalkyl” refers to a cycloalkyl group, as defined herein, having from 1 to 5, preferably 1-3 substituents independently selected from the same group of substituents as defined for substituted alkyl.

“Halo” or “halogen” refers to fluoro, chloro, bromo and iodo and preferably is either chloro or fluoro.

“Heteroaryl” refers to an aromatic group of from 1 to 10 ring carbon atoms and 1 to 4 ring heteroatoms selected from oxygen, nitrogen and sulfur within the ring. Such heteroaryl groups can have a single ring (e.g., pyridyl or furyl) or multiple condensed rings (e.g., indolizinyl or benzothienyl). Preferred heteroaryls include pyridyl, pyrrolyl, indolyl and furyl.

“Substituted heteroaryl” refers to heteroaryl groups, as defined above, which are substituted with from 1 to 3 substituents independently selected from the same group of substituents as defined for “substituted aryl”.

“Heteroaralkyl” refers to the group -alkyl-heteroaryl where alkyl and aryl are as defined herein. Such groups are exemplified by —CH₂-pyrid-4-yl.

“Heterocycle” or “heterocyclic” refers to a saturated or unsaturated group having a single ring or multiple condensed rings, from 1 to 10 ring carbon atoms and from 1 to 4 ring hetero atoms selected from nitrogen, sulfur or oxygen within the ring wherein, in fused ring systems, one or more of the rings can be aryl or heteroaryl.

“Substituted heterocyclic” refers to heterocyclic groups, as defined above, which are substituted with from 1 to 3 substituents independently selected from the group consisting of oxo (═O), thioxo (═S), plus the same group of substituents as defined for substituted aryl.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiazolidine, thiophene, benzo[b]thiophene, morpholino, thiomorpholino, piperidinyl, pyrrolidine, tetrahydrofuranyl, and the like.

It is understood that in all substituted groups defined above, polymers arrived at by defining substituents with further substitutions to themselves (e.g, substituted aryl having a substituted aryl group as a substituent which is itself substituted with a substituted aryl group, etc.) are not intended for inclusion herein. In such cases, the maximum number of such substituents is three, that is to say that each of the above definitions is constrained by a limitation that, for example, substituted aryl groups are limited to -substituted aryl-(substituted aryl)-substituted aryl.

Similarly, it is understood that the above definitions are not intended to include impossible substitution patterns (e.g., methyl substituted with 5 fluoro groups or a hydroxyl group alpha to ethenylic or acetylenic unsaturation). Such is well known to the skilled artisan.

“Pharmaceutically acceptable salt” refers to pharmaceutically acceptable salts of a compound of Formula I which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

Compound Preparation

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Second Edition, Wiley, N.Y., 1991, and references cited therein.

The compounds of this invention may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

Certain of the compounds of this invention contain vinyl unsaturation. Accordingly, if desired, such compounds can be prepared or isolated as pure cis- or trans-isomers or as enriched mixtures. All such isomers (and enriched mixtures) are included within the scope of this invention, unless otherwise indicated.

Specifically, one method for the preparation of the optionally substituted 3-(2′-sulfonamidophenyl)propionamides and related compounds (where W is N and q is one) is illustrated in Scheme (1) below: 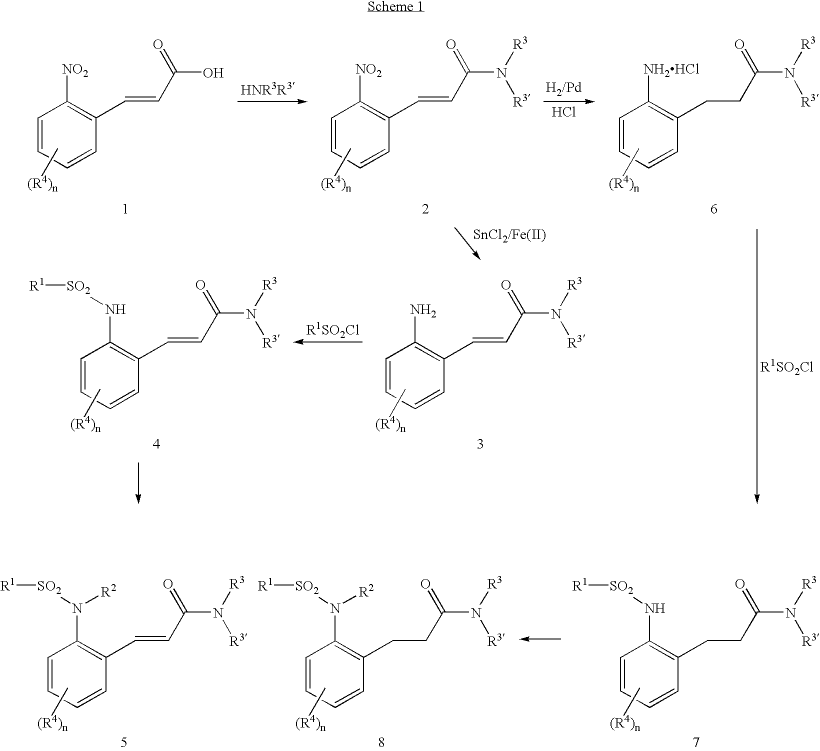
where R¹, R², R³, R^{3′}, R⁴ and n are as defined above.

Specifically, in Scheme 1, the carboxyl group of the optionally substituted 2-nitrocinnaminic acid, compound 1, (optionally substituted to the extent that n is not zero) is coupled under conventional amidation condition using a suitable amine, HNR³R^{3′}, to provide for the optionally substituted 2-nitrocinnaminamide, 2. This coupling reaction is typically conducted using well-known coupling reagents such as carbodiimides, BOP reagent (benzotriazol-1-yloxy-tris(dimethylamino)-phosphonium hexafluorophosphonate), HATU reagent [O-(7-azabenzotriazol-1-yl)-N,N,N′,N′-tetraethyluonium hexafluorophosphate], and the like. Suitable carbodiimides include, by way of example, dicyclohexylcarbodiimide (DCC), 1-(3-dimethylaminopropyl)-3-ethylcarbodiimide (EDC) and the like. If desired, polymer supported forms of carbodiimide coupling reagents may also be used including, for example, those described in Tetrahedron Letters, 34(48), 7685 (1993). Additionally, well-known coupling promoters, such as N-hydroxysuccinimide, 1-hydroxybenzotriazole and the like, may be used to facilitate the coupling reaction.

This coupling reaction is typically conducted by contacting the optionally substituted 2-nitrocinnaminic acid, 1, with about 1 to about 2 equivalents of the coupling reagent and at least one equivalent, preferably about 1 to about 1.2 equivalents of a suitable amine, HNR³R^{3′}, in an inert diluent, such as dichloromethane, chloroform, acetonitrile, tetrahydrofuran, N,N-dimethylformamide and the like. Generally, this reaction is conducted at a temperature ranging from about 0° C. to about 37° C. for about 12 to about 24 hours. Upon completion of the reaction, the optionally substituted 2-nitrocinnaminamide, compound 2, is recovered by conventional methods including neutralization, extraction, precipitation, chromatography, filtration, and the like.

Alternatively, the optionally substituted 2-nitrocinnaminic acid, compound 1, can be converted into an acid halide and the acid halide coupled with a suitable amine, HNR³R^{3′}, to provide for the optionally substituted 2-nitrocinnaminamide, compound 2. The acid halide can be prepared by contacting the optionally substituted 2-nitrocinnaminic acid, compound 1 with an inorganic acid halide, such as thionyl chloride, phosphorous trichloride, phosphorous tribromide or phosphorous pentachloride, or with oxalyl chloride under conventional conditions. Generally, this reaction is conducted using about 1 to 5 molar equivalents of the inorganic acid halide or oxalyl chloride, either neat or in an inert solvent, such as dichloromethane or carbon tetrachloride, at temperature in the range of about 0° C. to about 80° C. for about 1 to about 48 hours. A catalyst, such as DMF, may also be used in this reaction.

The acid halide is then contacted with at least one equivalent, preferably about 1.1 to about 1.5 equivalents, of the suitable amine, HNR³R^{3′}, in an inert diluent, such as dichloromethane, at a temperature ranging from about −70° C. to about 40° C. for about 1 to about 24 hours. Preferably, this reaction is conducted in the presence of a suitable base to scavenge the acid generated during the reaction. Suitable bases include, by way of example, tertiary amines, such as triethylamine, diisopropylethylamine, N-methylmorpholine and the like.

In one route illustrated in Scheme 1, the nitro group of the optionally substituted 2-nitrocinnaminamide, compound 2, is selectively reduced while retaining vinyl unsaturation in the side chain of the cinnaminamide to provide for the optionally substituted 2-aminocinnaminamide, compound 3. In this scheme, mild reduction conditions are employed which utilize either tin dichloride or Fe(II) in the presence of HCl in ether, in acetic acid as a solvent for 1 to 12 hours at from about 30° C. to about 70° C. The vinyl unsaturation in the side chain of the cinnaminamide remains trans throughout this transformation.

Upon completion of the reaction, the optionally substituted 2-amino-cinnaminamide, compound 3, is recovered by conventional methods including neutralization, extraction, precipitation, chromatography, filtration, and the like.

The optionally substituted 2-aminocinnaninamide, compound 3, is then sulfonated to provide for the optionally substituted 2-(N-sulfonamide) cinnaminamide, compound 4. The sulfonation reaction is typically effected by contacting compound 3 with about a stoichiometric amount, or slight excess, of the desired sulfonyl chloride, R¹SO₂Cl in the presence of a scavenger base, such as pyridine, and the like in an inert diluent. The reaction is typically conducted at temperatures in the range of about 0° C. to about room temperature for a period of time to effect sulfonation, which is typically 2 to 12 hours. Suitable inert solvents which can be used include, dichloromethane, and the like. The resulting optionally substituted 2-(N-sulfonamide) cinnaminamide, compound 4, can be recovered by conventional methods, such as neutralization, chromatography, filtration, crystallization, and the like, or can be used in the next step without purification or isolation.

For compounds of this invention where R² is alkyl or cycloalkyl, the optionally substituted 2-(N-sulfonamide) cinnaminamide, compound 4, is next reacted with a stoichiometric equivalent or slight excess of an alkyl iodide, or a cycloalkyl iodide under suitable conditions to provide for compound 5 (where R² is alkyl or cycloalkyl). The reaction (sometimes generically referred to herein as the alkylation reaction) is preferably conducted in the presence of a suitable base such as potassium carbonate, sodium carbonate, triethylamine, and the like to scavenge the acid generated during the reaction. The reaction is conducted in a suitable inert diluent such as acetone, dimethylformamide and the like at a temperature typically of from about 20° C. to about 75° C. for a period of typically from about 3 to about 12 hours. Alternatively, aryl boronic acid, heteroaryl boronic acid or heterocyclic boronic acid can be reacted with compound 4 in the presence of CuI/base in solvents such as dichloromethane, THF or the like to form compound 5 (where R² is aryl, heteroaryl or heterocyclic) and the vinyl unsaturation in compound 5 is in the trans orientation. The resulting optionally substituted 2-(N-substituted sulfonamide) cinnaminamide, compound 5, can be recovered by conventional methods, such as neutralization, chromatography, filtration, crystallization, and the like, or can be used in the next step without purification or isolation.

One method that can be used to obtain the cis-isomer of compound 5, is to do a cis-trans isomerization reaction using conventional conditions. The cis- and trans-compounds can then be purified using standard separation and collection techniques.

In another route illustrated in Scheme 1, the nitro group of the optionally substituted 2-nitrocinnaminamide, compound 2, is non-selectively hydrogenated relative to the vinyl unsaturation to provide for the optionally substituted 3-[2′-aminophenyl]propionamide compound 6. This reaction is conducted under conventional hydrogenation conditions employing elevated pressures of hydrogen in the presence of a suitable hydrogenation catalyst such as platinum oxide, palladium and the like in a suitable solvent such as ethyl acetate, methanol, and the like. The reaction is preferably conducted in an acidic environment such as 1N HCl and a particularly preferred solvent for this reaction is 1N HCl in ether. The reaction is conducted at a temperature typically of from about 15° C. to about 40° C. for a period of typically from about 1 to about 3 hours. The resulting optionally substituted 3-[2′-aminophenyl]propionamide compound 6 can be recovered by conventional methods, such as neutralization, chromatography, filtration, crystallization, and the like, or can be used in the next step without purification or isolation.

Subsequently, sulfonation and optional alkylation of the optionally substituted 3-[2′-aminophenyl]propionamide compound 6 proceeds in the manner described above to provide for either the optionally substituted 3-[2′-(phenylsulfonamido)phenyl]propionamide compound 7 or the optionally substituted 3-[2′-(phenyl-N-substituted sulfonylamido)phenyl]propionamide compound 8.

Alternatively, in Scheme 1, the amine compounds 3 and 6 can be treated reacted under conventional reductive amination conditions to provide for another route for the synthesis of a variety of R² alkyl or substituted alkyl variables in compounds 5 and 8 which are compounds of this invention. This alternative scheme is illustrated in Scheme 2 below which employs compound 6 for illustrative purposes only: 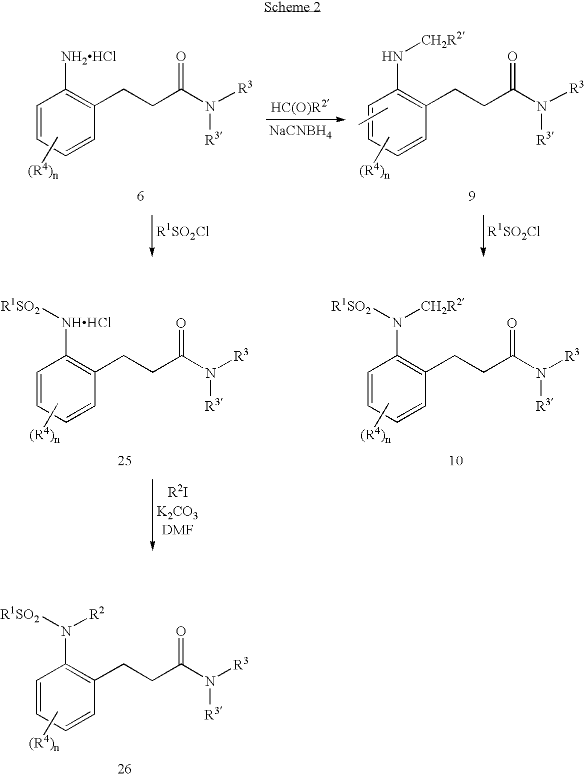
where R¹, R³, R^{3′}, R⁴ and n are as defined above.

In one route of Scheme 2, optionally substituted 3-[2′-(phenyl-N-substituted sulfonylamido)phenyl]propionamide compounds are prepared by first alkylating compound 6, followed by sulfonylation using methods that prevent alkylation to quaternary amines. More specifically in the first step, the amine of compound 6 is contacted with a suitable aldehyde, HC(O)R^{2′}, where R^{2′} is selected from allyl, aryl, aralkyl, heteroaryl or heteroaralkyl, in the presence of a suitable reducing agent such as sodium cyanoborohydride under conventional reductive amination conditions to provide for the optionally substituted 3-[2′-N—(—CH₂—R^{2′}) amino]phenyl propionamide, compound 9. The reaction is typically conducted in an inert solvent such as methanol or ethanol at a temperature of from about 0° C. to about 60° C., although preferably at room temperature. The reaction is continued until substantial completion which typically occurs within about 1 to 24 hours. The resulting product can be recovered by conventional methods, such as solvent stripping, chromatography, filtration, crystallization, and the like, or can be used in the next step without purification and/or isolation.

The optionally substituted 3-[2′-N—(—CH₂—R^{2′}) amino]phenyl propionamide, compound 9, is then sulfonated in the manner described above to provide for the optionally substituted 3-[2-phenyl-N-substituted sulfonylamidophenyl]propionamide compound 10, including a catalytic amount of DMAP.

In another route illustrated in Scheme 2, sulfonylation of compound 6 as described above is followed by reaction of the resulting compound 25 with the appropriate alkyliodide and potassium carbonate in DMF to provide for compound 26. The reaction is run at about 25° C. to about 45° C. for 1 to 5 hours, or until the reaction is substantially complete. The resulting product, compound 26, can be recovered by conventional methods, such as solvent stripping, chromatography, filtration, crystallization, and the like, or can be used in the next step without purification and/or isolation.

Scheme 3, below, illustrates an alternative synthetic pathway to the formation of compound 8. In addition, this pathway also provides for the synthesis of the optionally substituted 3-(2′-sulfonamidophenyl)propargylamides and related compounds (where W is N and q is one). 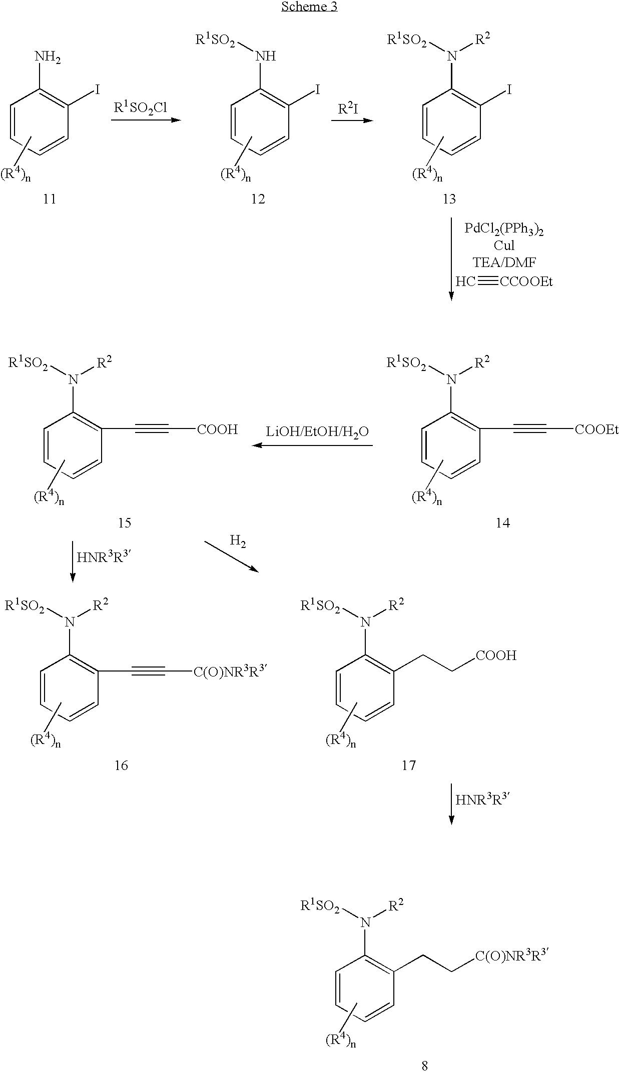
where R¹, R², R³, R^{3′}, R⁴ and n are as defined above.

Specifically, in Scheme 3, optionally substituted 2-aminoiodobenzene, compound 11, is sulfonated to provide for the optionally substituted 2-(N-sulfonamido)iodobenzene, compound 12. Subsequently and optionally, the nitrogen atom of compound 12 is then alkylated, (arylated, heteroarylated), etc. by contacting compound 12 with a stoichiometric amount or slight excess of an alkyl iodide,( aryl boronic acid, heteroaryl boronic acid), etc. Sulfonation, alkylation and recovery is conducted in a manner described above in Scheme 1.

Coupling of compound 13 and commercially available ethyl propiolate using bis(triphenylphosphine)palladium dichloride, copper iodide and triethylamine in dimethylformamide provides for the optionally substituted ethyl 3-[2-sulfonamidophenyl]propiolate, compound 14.

Conventional hydrolysis of the ester group in the optionally substituted ethyl 3-[2′-(sulfonamido)phenyl]propiolate, compound 14, using, for example, lithium hydroxide in a solvent mixture of ethanol and water provides for the optionally substituted 3-[2′-(sulfonamido)phenyl]propiolic acid, compound 15. The reaction is typically conducted at a temperature of from about 0° C. to about 60° C., although preferably at room temperature. The reaction is continued until substantial completion which typically occurs within about 1 to 24 hours. The resulting product can be recovered by conventional methods, such as solvent stripping, neutralization, chromatography, filtration, crystallization, chromatography, and the like, or can be used in the next step without purification and/or isolation.

In one embodiment, the carboxyl group of the optionally substituted 3-[2′-(sulfonamido)phenyl]propiolic acid, compound 15, is coupled under conventional amidation condition using a suitable amine, HNR³R^{3′}, to provide for the optionally substituted 3-[(2′-sulfonamido)phenyl]propargylamide, compound 16. Coupling proceeds in the manner described above in Scheme 1 and the resulting product can be recovered by conventional methods, such as solvent stripping, neutralization, chromatography, filtration, crystallization, chromatography, and the like.

In another embodiment, hydrogenation of the acetylenic unsaturation in the optionally substituted 3-[2′-(sulfonamido)phenyl]propiolic acid, compound 15, provides for the optionally substituted 3-[2′-(sulfonamido)phenyl]propionic acid, compound 17. This reaction is conducted under conventional hydrogenation conditions employing elevated pressures of hydrogen in the presence of a suitable hydrogenation catalyst such as platinum oxide, palladium and the like in a suitable solvent such as ethyl acetate, methanol, and the like. The reaction is conducted at a temperature typically of from about 15° C. to about 40° C. for a period of typically from about 1 to 6 hours. Resulting compound 17 can be recovered by conventional methods, such as neutralization, chromatography, filtration, crystallization, and the like, or can be used in the next step without purification or isolation.

The carboxyl group of compound 17 is coupled under conventional amidation condition using a suitable amine, HNR³R^{3′}, to provide for the optionally substituted 3-[2′-(sulfonamido)phenyl]propionamide, compound 8. Coupling proceeds in the manner described above in Scheme 1 and the resulting product can be recovered by conventional methods, such as solvent stripping, neutralization, chromatography, filtration, crystallization, chromatography, and the like.

Alternatively, the optionally substituted 2-aminoiodobenzene, compound 11, can be reacted under conventional reductive amination conditions to provide for another route for the synthesis of a variety of R² alkyl or substituted alkyl variables in compound 13. This alternative scheme is illustrated in Scheme 4 below:

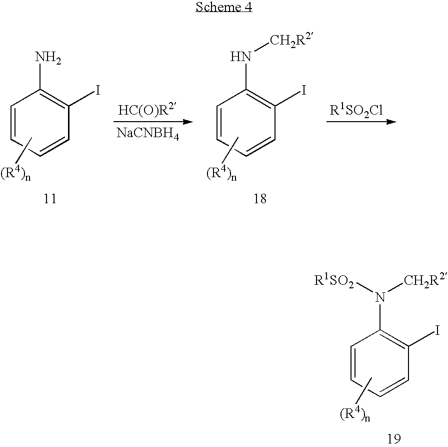

In Scheme 4, amine 11 is contacted with a suitable aldehyde, HC(O)R^{2′}, where R^{2′} is selected from alkyl, aryl, aralkyl, heteroaryl or heteroaralkyl, in the presence of a suitable reducing agent such as sodium cyanoborohydride under conventional reductive amination conditions to provide for the optionally substituted 2-(-NHCH₂R^{2′})iodobenzene, compound 18. The reaction is typically conducted in an inert solvent such as methanol or ethanol at a temperature of from about 0° C. to about 60° C., although preferably at room temperature with a few drops of acetic acid. The reaction is continued until substantial completion which typically occurs within about 1 to 24 hours. The resulting product can be recovered by conventional methods, such as solvent stripping, chromatography, filtration, crystallization, and the like, or can be used in the next step without purification and/or isolation.

Compound 18, is then sulfonated in the manner described above to provide for the optionally substituted 2-(N-substituted sulfonylamido)iodobenzene, compound 19 which can be used in-place of compound 13 in Scheme 3 to provide for compounds of this invention.

In still another alternative embodiment, optionally substituted 3-[2′-(phenyl-N-methylsulfonylamido)phenyl]propionamide, compound 24, can be prepared in a manner illustrated in Scheme 5 below: 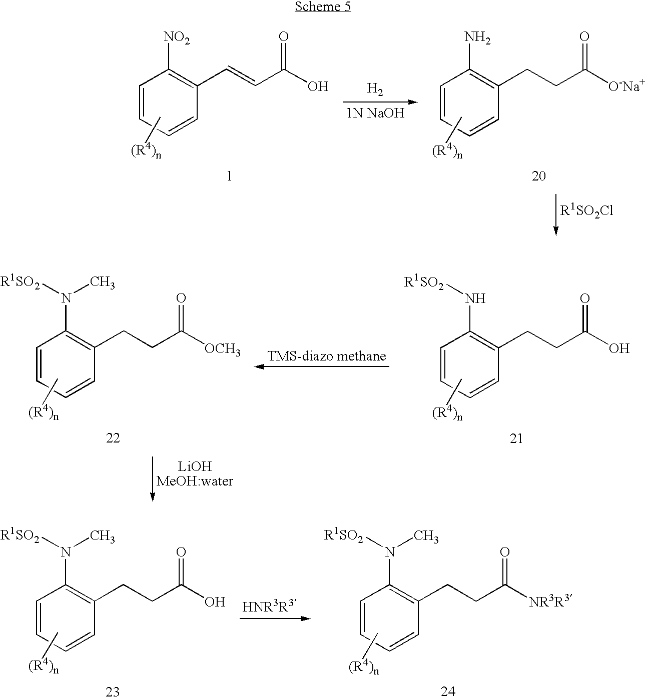
where R¹, R³, R^{3′}, R⁴ and n are as defined above and R² is methyl.

In Scheme 5, conventional hydrogenation of the optionally substituted 2-nitrocinnaminic acid, 1, yields the corresponding optionally substituted sodium 3-(2-aminophenyl)propionate, 20. The reaction is conducted under conventional hydrogenation conditions employing elevated pressures of hydrogen in the presence of a suitable hydrogenation catalyst such as platinum oxide, palladium and the like in a suitable solvent such as ethyl acetate, methanol, and the like. The reaction is conducted in a basic environment such as 1N NaOH in order to inhibit cyclization of the acid with the amino group. The reaction is conducted at a temperature typically of from about 15° C. to about 40° C. for a period of typically from about 1 to 8 hours. The resulting product, compound 20, can be recovered by conventional methods, such as neutralization, chromatography, filtration, crystallization, and the like, or can be used in the next step without purification or isolation.

Compound 20, is then sulfonated in the manner described above to provide for the optionally substituted 3-[2′-(sulfonylamido)phenyl]propionic acid, compound 21, in the manner described above in Scheme 1.

Methylation of the optionally substituted 3-[2′-(sulfonylamido)phenyl]propionic acid, compound 21, is achieved by reaction with trimethylsilyl diazomethane to provide for the optionally substituted 3-[2′-(N-methylsulfonyl-amido)phenyl]propionic acid, compound 22. The reaction is typically conducted in an inert solvent such as dichloromethane at a temperature of from about 0° C. to about 40° C. The reaction is continued until substantial completion which typically occurs within about 1 to about 8 hours. The resulting product can be recovered by conventional methods, such as solvent stripping, chromatography, filtration, crystallization, and the like, or can be used in the next step without purification and/or isolation.

Hydrolysis of compound 22 using standard conditions such as LiOH in MeOH and water will provide compound 23, which can be amidated in the manner described above in Scheme 1 to provide for compound 24.

The starting materials for the above reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

Sulfonyl chlorides of the formula R¹SO₂Cl as employed in the above reaction are either known compounds or compounds that can be prepared from known compounds by conventional synthetic procedures. Such compounds are typically prepared from the corresponding sulfonic acid, i.e., from compounds of the formula R¹—SO₃H where R¹ is as defined above, using phosphorous trichloride and phosphorous pentachloride. This reaction is generally conducted by contacting the sulfonic acid with about 2 to 5 molar equivalents of phosphorous trichloride and phosphorous pentachloride, either neat or in an inert solvent, such as dichloromethane, at temperature in the range of about 0° C. to about 80° C. for about 1 to about 48 hours to afford the sulfonyl chloride. Alternatively, the sulfonyl chlorides can be prepared from the corresponding thiol compound, i.e., from compounds of the formula R¹—SH where R is as defined herein, by treating the thiol with chlorine (Cl₂) and water under conventional reaction conditions.

Examples of sulfonyl chlorides suitable for use in this invention include, but are not limited to, benzenesulfonyl chloride, 1-naphthalenesulfonyl chloride, 2-naphthalenesulfonyl chloride, p-toluenesulfonyl chloride, α-toluenesulfonyl chloride, 4-acetamidobenzenesulfonyl chloride, 4-amidinobenzenesulfonyl chloride, 4-tert-butylbenzenesulfonyl chloride, 4-bromobenzenesulfonyl chloride, 2-carboxybenzenesulfonyl chloride, 4-cyanobenzenesulfonyl chloride, 3,4-dichlorobenzenesulfonyl chloride, 3,5-dichlorobenzenesulfonyl chloride, 3,4-dimethoxybenzenesulfonyl chloride, 3,5-ditrifluoromethylbenzenesulfonyl chloride, 4-fluorobenzenesulfonyl chloride, 4-methoxybenzenesulfonyl chloride, 2-methoxycarbonylbenzene-sulfonyl chloride, 4-methylamidobenzenesulfonyl chloride, 4-nitrobenzene-sulfonyl chloride, 4-thioamidobenzenesulfonyl chloride, 4-trifluoromethyl-benzenesulfonyl chloride, 4-trifluoromethoxybenzenesulfonyl chloride, 2,4,6-trimethylbenzenesulfonyl chloride, 2-phenylethanesulfonyl chloride, 2-thiophenesulfonyl chloride, 5-chloro-2-thiophenesulfonyl chloride, 2,5-dichloro-4-thiophenesulfonyl chloride, 2-thiazolesulfonyl chloride, 2-methyl-4-thiazolesulfonyl chloride, 1-methyl-4-imidazolesulfonyl chloride, 1-methyl-4-pyrazolesulfonyl chloride, 5-chloro-1,3-dimethyl-4-pyrazolesulfonyl chloride, 3-pyridinesulfonyl chloride, 2-pyrimidinesulfonyl chloride and the like. If desired, a sulfonyl fluoride, sulfonyl bromide or sulfonic acid anhydride may be used in place of the sulfonyl chloride in the above reactions.

Amines of the formula HNR³R^{3′} are either commercially available or can be prepared by methods well known in the art some of which are illustrated in the examples below.

2-Nitrocinnaminic acid is commercially available and methods for forming optional substitution on the phenyl group thereof acid are well known in the art.

Similarly, 2-iodoaniline is commercially available and methods for forming optional substitution on the phenyl group thereof are well known in the art.

In some cases it may be more convenient to prepare a given product compound or intermediate by preparing it from another product of Formula I or intermediate, by applying known synthesis procedures. For example, as noted above, conversion of compounds where R⁷ is hydrogen into other compounds where R⁷ is another moiety can be accomplished after formation of compounds within the scope of Formula I above.

Pharmaceutical Formulations

When employed as pharmaceuticals, the compounds of Formula I and II are usually administered in the form of pharmaceutical compositions. These compounds can be administered by a variety of routes including oral, rectal, transdermal, subcutaneous, intravenous, intramuscular, and intranasal. These compounds are effective as both injectable and oral compositions. Such compositions are prepared in a manner well known in the pharmaceutical art and comprise at least one active compound.

This invention also includes pharmaceutical compositions which contain, as the active ingredient, one or more of the compounds of formula I and II above associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound is effective over a wide dosage range and is generally administered in a pharmaceutically effective amount. It, will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, preferably orally or nasally, from devices which deliver the formulation in an appropriate manner.

The following formulation examples illustrate the pharmaceutical compositions of the present invention.

### Formulation Example 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules in 340 mg quantities.

### Formulation Example 2

A tablet formula is prepared using the ingredients below:

| | Quantity |
|---|---|
| Ingredient | (mg/capsule) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### Formulation Example 3

A dry powder inhaler formulation is prepared containing the following components:

| Ingredient | Weight % |
|---|---|
| Active Ingredient | 5 |
| Lactose | 95 |

The active mixture is mixed with the lactose and the mixture is added to a dry powder inhaling appliance.

### Formulation Example 4

Tablets, each containing 30 mg of active ingredient, are prepared as follows:

| | Quantity | |
|---|---|---|
| Ingredient | (mg/tablet) | |
| Active Ingredient | 30.0 | mg |
| Starch | 45.0 | mg |
| Microcrystalline cellulose | 35.0 | mg |
| Polyvinylpyrrolidone | 4.0 | mg |
| (as 10% solution in water) | | |
| Sodium carboxymethyl starch | 4.5 | mg |
| Magnesium stearate | 0.5 | mg |
| Talc | 1.0 | mg |
| Total | 120 | mg |

The active ingredient, starch and cellulose are passed through a No. 20 mesh U.S. sieve and mixed thoroughly. The solution of polyvinyl-pyrrolidone is mixed with the resultant powders, which are then passed through a 16 mesh U.S. sieve. The granules so produced are dried at 50° C. to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 150 mg.

### Formulation Example 5

Capsules, each containing 40 mg of medicament are made as follows:

| | Quantity |
|---|---|
| Ingredient | (mg/capsule) |
| Active Ingredient | 40.0 mg |
| Starch | 109.0 mg |
| Magnesium stearate | 1.0 mg |
| Total | 150.0 mg |

The active ingredient, cellulose, starch, and magnesium stearate are blended, passed through a No. 20 mesh U.S. sieve, and filled into hard gelatin capsules in 150 mg quantities.

### Formulation Example 6

Suppositories, each containing 25 mg of active ingredient are made as follows:

| Ingredient | Amount |
|---|---|
| Active Ingredient | 25 mg |
| Saturated fatty acid glycerides to | 2,000 mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation Example 7

Suspensions, each containing 50 mg of medicament per 5.0 mL dose are made as follows:

| Ingredient | Amount | |
|---|---|---|
| Active Ingredient | 50.0 | mg |
| Xanthan gum | 4.0 | mg |
| Sodium carboxymethyl cellulose (11%) | 50.0 | mg |
| Microcrystalline cellulose (89%) | | |
| Sucrose | 1.75 | g |
| Sodium benzoate | 10.0 | mg |
| Flavor and Color | q.v. | |
| Purified water to | 5.0 | mL |

The medicament, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation Example 8

| | Quantity |
|---|---|
| Ingredient | (mg/capsule) |
| Active Ingredient | 15.0 mg |
| Starch | 407.0 mg |
| Magnesium stearate | 3.0 mg |
| Total | 425.0 mg |

The active ingredient, cellulose, starch, and magnesium stearate are blended, passed through a No. 20 mesh U.S. sieve, and filled into hard gelatin capsules in 560 mg quantities.

### Formulation Example 9

An intravenous formulation may be prepared as follows:

| Ingredient | Quantity | |
|---|---|---|
| Active Ingredient | 250.0 | mg |
| Isotonic saline | 1000 | mL |

### Formulation Example 10

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, which is incorporated herein by reference in its entirety. Such patches may be constructed for continuous, pulsatile, or on demand delivery of pharmaceutical agents.

When it is desirable or necessary to introduce the pharmaceutical composition to the brain, either direct or indirect techniques may be employed. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 which is incorporated herein by reference in its entirety.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

Utility

The compounds of this invention are bradykinin antagonists and therefore are suitable for use in blocking or ameliorating pain as well as hyperalgesia in mammals. Pain blocked or ameliorated by the compounds of this invention include, for example, pain associated with surgical procedures, burns, trauma, migraine, and the like.

The compounds of this invention are also useful in the treatment of disease conditions in a mammal which are mediated at least in part by bradykinin. Examples of such disease conditions include asthma, rhinitis, premature labor, inflammatory arthritis, inflammatory bowel disease, endotoxic shock related to bacterial infections, central nervous system injury, back pain, neuropathic pain, spinal cord injury and the like.

As noted above, the compounds of this invention are typically administered to the mammal in the form of a pharmaceutical composition. Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985).

In order to enhance serum half-life, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka, et al., U.S. Pat. Nos. 4,235,871, 4,501,728 and 4,837,028 each of which is incorporated herein by reference.

The amount administered to the patient will vary depending upon what is being administered, the purpose of the administration, such as prophylaxis or therapy, the state of the patient, the manner of administration, and the like all of which are within the skill of the attending clinician. In therapeutic applications, compositions are administered to a patient already suffering from a disease in an amount sufficient to cure or at least partially arrest the symptoms of the disease and its complications. An amount adequate to accomplish this is defined as “therapeutically effective dose.” Amounts effective for this use will depend on the disease condition being treated as well as by the judgment of the attending clinician depending upon factors such as the severity of the inflammation, the age, weight and general condition of the patient, and the like.

The compositions administered to a patient are in the form of pharmaceutical compositions described above. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention will vary according to, for example, the particular use for which the treatment is made, the manner of administration of the compound, the health and condition of the patient, and the judgment of the prescribing physician. For example, for intravenous administration, the dose will typically be in the range of about 20 μg to about 500 μg per kilogram body weight, preferably about 100 μg to about 300 μg per kilogram body weight. Suitable dosage ranges for intranasal administration are generally about 0.1 pg to 1 mg per kilogram body weight. Effective doses can be extrapolated from dose-response curves derived from in vitro or animal model test systems.

In addition to the above, the esters and thioesters of formula I are useful intermediates in the preparation of the amides of formula I (W═N).

The following synthetic and biological examples are offered to illustrate this invention and are not to be construed in any way as limiting the scope of this invention. Unless otherwise stated, all temperatures are in degrees Celsius.

### EXAMPLES

In the examples below, the following abbreviations have the following meanings. If an abbreviation is not defined, it has its generally accepted meaning.
Boc=t-butoxycarbonyl
brd=broad doublet
brm=broad multiplet
brt=broad triplet
bs=broad singlet
dba=dibenzyledene acetone
dd=doublet of doublets
DIAD=diisopropyl azo dicarboxylate
DIEA=diisopropylethyl amine
DMAP=4-N,N-dimethylaminopyridine
DME=dimethoxyethane
DMF=N,N-dimethylformamide
DPPA=diphenylphosphoryl azide
dppf=1,1′-bis(diphenylphosphino)ferrocene
dt=doublet of triplets
EDCI=1-(3-dimethylaminopropyl)-3-ethylcarbodiimide hydrochloride
EtOH=ethanol
eq.=equivalents
g=gram
h=hours
HATU=O-(7-azabenzotriazol-1-yl)-N,N,N′,N′-tetra-ethyluonium hexafluorophosphate
HOAc=acetic acid
HOBT=1-hydroxybenzothiazole hydrate
HPLC=high performance liquid chromatography
MS=mass spectroscopy
MeOH=methanol
m=multiplet
M=molar
mg=milligram
min.=minutes
mL=milliliter
mmol=millimolar
NMR=nuclear magnetic resonance
N=normal
OAc=acetate
psi=pounds per square inch
q=quartet
rt=room temperature
R₁=retention time
s=singlet
t=triplet
TEA=triethylamine
TFA=trifluoroacetic acid
THF=tetrahydrofuran
μL=microliters

In the following examples and procedures, the term “Aldrich” indicates that the compound or reagent used in the procedure is commercially available from Aldrich Chemical Company, Inc., Milwaukee, Wis. 53233 USA; the term “Sigma” indicates that the compound or reagent is commercially available from Sigma, St. Louis Mo. 63178 USA and the term “TCI” indicates that the compound or reagent is commercially available from TCI America, Portland Oreg. 97203; the term “Frontier” or “Frontier Scientific” indicates that the compound or reagent is commercially available from Frontier Scientific, Utah, USA; “Bachem” indicates that the compound or reagent is commercially available from Bachem, Torrance, Calif., USA; the term “Lancaster” indicates that the compound or reagent is commercially available from Lancaster Synthesis, Inc., P.O. Box 100 Windham, N.H. 03087 USA; the term “Peptech” indicates that the compound or reagent is commercially available from Peptech Corporation, Cambridge, Mass. USA.

The following general procedures illustrate general synthetic pathways for preparing amine intermediates useful in preparing compounds of Formula I or for modifying the acetamide group on compounds of formula I.

### General Procedure A

### GENERAL PROCEDURE FOR THE PREPARATION OF 1,2,3,6-TETRAHYDRO-N-ALKYLPYRIDINE DERIVATIVES

A suitable starting material comprising a 2-acetamide group on an appropriate propionamide compound having a pyridine functionality attached thereto (2.92 mmol) is added to dry DMF (15 mL) and is heated with a heat-gun (if required) to form a clear solution which is then cooled to rt. Methyl iodide (5 mL, excess) is added thereto and stirring is continued for 18 h at rt. Excess DMF is removed under reduced pressure and the pyridinium salt formed is taken to the next step without further purification. The methyl iodide salt is dissolved in methanol (25 mL) and NaBH₄ (13.78 mmol) is added to it and stirred for 1 h. Excess MeOH is removed and water (50 mL) is added to the crude product and sonicated for 10 min. A solid product containing the 1,2,3,6-tetrahydro-N-methylpyridine group is filtered off or extracted with CH₂Cl₂ and used in the next step without further purification.

The remaining double bond in the 1,2,3,6-tetrahydro-N-methylpyridine group can optionally be hydrogenated to provide for the N-methylpiperidin-4-yl derivative.

### General Procedure B

### GENERAL PROCEDURE FOR THE PREPARATION OF CYCLOPROPYLPIPERIDINYL PROPIONAMIDES

3-[2′-{(4″-chloro-2″,5″-dimethylbenzene)-N′-methylsulfonamido)phenyl]-N-[(2-pyrid-4-yl)eth-1-yl]propionamide, which can be prepared by amidation of the corresponding carboxylic acid with 2-(2-aminoethyl)pyridine (TCI) in the manner described above is hydrogenated in the presence of platinum oxide (PtO₂) in methanol to provide for the corresponding piperidinyl derivative.

Sodium cyanoborohydride (1.5 mmol) is then added to a stirred solution of the piperidinyl derivative (1 mmol), with 1-ethoxy-1-trimethylsiloxy cyclopropane (1 mmol) (Aldrich) and AcOH (1 mmol) in MeOH (20 mL) at rt. After being stirred at rt, the reaction mixture is refluxed for 18 h. The excess solvent is removed and washed with saturated NaHCO₃ solution. The aqueous solution is extracted with CH₂Cl₂(2×100 mL). The combined organic layers are dried and concentrated. The resulting residue is then purified by silica gel column chromatography to afford the N-yclopropylpiperidinylethyl propionamide derivative.

### General Procedure C

### GENERAL PROCEDURE FOR THE PREPARATION OF N-PHENYLPIPERIDINYLETHYL PROPIONAMIDES

Triphenylbismuth diacetate (Ph₃Bi(OAc)₂) (1.2 eq.) and Cu(OAc)₂ (0.12 eq.) are added to a stirred solution of an appropriate N-2-(piperidin4-ylethyl)propioamide compound (1 mmol) in dichloromethane at rt and stirred for 18 h. The reaction mixture is partitioned between dichloromethane (50 mL) and water (50 mL) and stirred for 2 h. The organic layer is separated, dried and concentrated. The residue was chromatographed on silica gel affording the N-[2-(N-phenyl-piperidin-4yl)ethyl]propionamide derivative.

### General Procedure D

### GENERAL PROCEDURE FOR THE PREPARATION OF N-PYRIDYLPIPERIDINYLETHYL PROPIONAMIDES

A solution of an appropriate N-2-(piperidin-4-ylethyl)propioamide compound (0.1 mmol) and 4-chloropyridine (excess) in EtOH (5 mL) is heated in a sealed tube at 110° C. for 16 h. Excess solvent is removed and the residue purified by preparative HPLC (acetonitrile-water-0.1% TFA) and the N-[2-({N-pyrid-4-yl}piperidin-4-yl)ethyl]propionamide derivative is isolated as the TFA salt.

### General Procedure E

### GENERAL PROCEDURE FOR REMOVAL OF BOC PROTECTING GROUPS FROM AMINO GROUPS

To a stirred solution of Boc-amine (0.01 mol) in dry ethyl acetate (25 mL) at 0° C., HCl gas is bubbled for 15 min. The reaction solution is stirred for 5 h at rt after which the HCl salt is recovered by filtration. The HCl salt is used in the next step without further purification.

### General Procedure F

### GENERAL PROCEDURE FOR REMOVAL OF BOC PROTECTING GROUPS FROM AMINO GROUPS

HCl gas is bubbled for 2 h into a solution of Boc amino acid in dry MeOH (100 mL) at rt. The reaction solution is stirred for 18 h at rt after which the product is recovered upon solvent removal. The HCl salt is used in the next step without further purification.

### General Procedure G

### GENERAL PROCEDURE FOR CONVERSION OF A CYANOPHENYL GROUP TO A 4,5-DIHYDROIMIDAZOL-2-YLPHENYL GROUP

An N-[2-(p-cyanophenyl)ethyl]propionamide compound (1.57 mmol) which can be prepared in a manner as described herein is dissolved in a solution of Et₃N/pyridine (6 mL/60 mL) at rt. H₂S is bubbled through for 15 min. at rt. The reaction mixture is then capped and stirred at rt overnight. The solvent mixture is removed under reduced pressure and the resulting residue is then dissolved in a mixture of acetone/iodomethane (60 mL:5 mL). The solution is heated to reflux for 1.5 h whereupon the solvent is removed under reduced pressure. The crude material is dissolved in dry MeOH (15 mL), with Et₃N (1.0 eq.; 220 L) and ethylenediamine (1.1 eq.; 120 L). The solution is refluxed for 2 days. The solvent is evaporated under reduced pressure. The crude material can be purified by reverse phase HPLC (acetonitrile/water-0.1% TFA), and the resulting product isolated.

The process set forth in General Procedure H below is illustrated in the following reaction scheme:

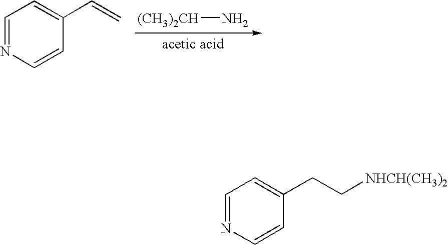

### General Procedure H

### GENERAL PROCEDURE FOR CONVERSION OF A VINYLPYRIDINE GROUP TO A 2-AMINOETHYLPYRIDINE GROUP

4-Vinyl pyridine (1.6 mL; 15 mmol) is dissolved in acetic acid (12.5 mmol; 0.72 mL) and isopropylamine (12.5 mmol; 1.06 mL). The reaction mixture is refluxed for 6 h. The solvent is evaporated under reduced pressure. To the resulting solid is added EtOAc as well as saturated NaHCO₃. The organic layer is isolated, dried over MgSO₄. The solvent is removed under reduced pressure. The desired material is isolated as a foam. H¹ NMR (CDCl₃) δ=8.4 (m, 2H); 7.05 (m, 2H); 2.75 (m, 2H); 2.65 (m, 3H); 0.99 (d, 6H). C¹³ NMR (CDCl₃) 149.87; 149.54; 149.09; 123.93; 48.19; 47.20; 35.56; 22.43. MS (API-ES)=165 (M+H).

The processes set forth in General Procedure I below are illustrated in the following reaction scheme:

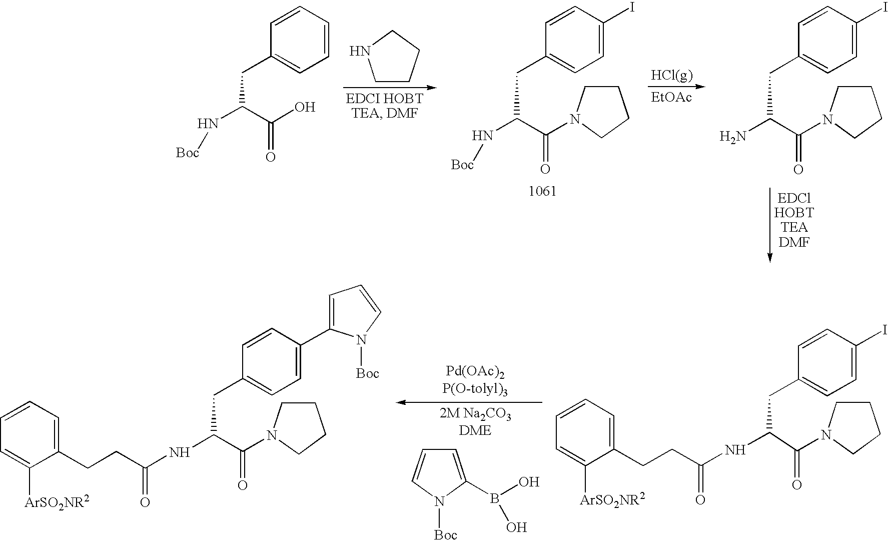

### General Procedure I

### GENERAL PROCEDURE FOR FORMING A HETEROARYL SUBSTITUENT ON A PHENYL GROUP

(D)-N-t-butoxycarbonyl-p-iodophenylalanine can be prepared by Boc protecting the commercially available p-iodophenylalanine (Aldrich). This compound can then be amidated by reaction with pyrrolidine using conventional coupling procedures to provide for 1-(R)-[1-(t-butoxycarbonyl-amino)-1-(pyrrolidin-1-ylcarbonyl)-2-(4-iodophenyl)]ethane and this amino acid derivative is sometimes referred to herein as compound 1061.

Removal of the Boc protecting group and coupling with a suitable 3-[2′-sulfonylamido)phenyl]propionic acid compound in a manner similar to that described herein affords the N-substituted propionamide compound.

This compound (0.34 mmol) is dissolved in dry DME (6 mL) under nitrogen. To this is added Pd(OAc)₂ (0.1 eq.), P(O-tolyl)₃ (0.1 eq.), 2M Na₂CO₃ (1.7 mL) and 1-(t-butoxycarbonyl)pyrrole-2-boronic acid (2 eq.) (Frontier Scientific). The reaction mixture is stirred overnight at 80° C. The solvent is removed under vacuum and EtOAc (20 mL) is added. The organic layer is washed with H₂O (10 mL, 2×), brine (10 mL, 1×) and dried over Na₂SO₄. Upon filtration, the solvent is removed under vacuum and the desired product can be purified on column chromatography (silica gel).

Optionally and subsequently, the Boc protecting group on the pyrrolyl group can be removed in the manner described above.

The processes set forth in General Procedure J below are illustrated in the following reaction scheme:

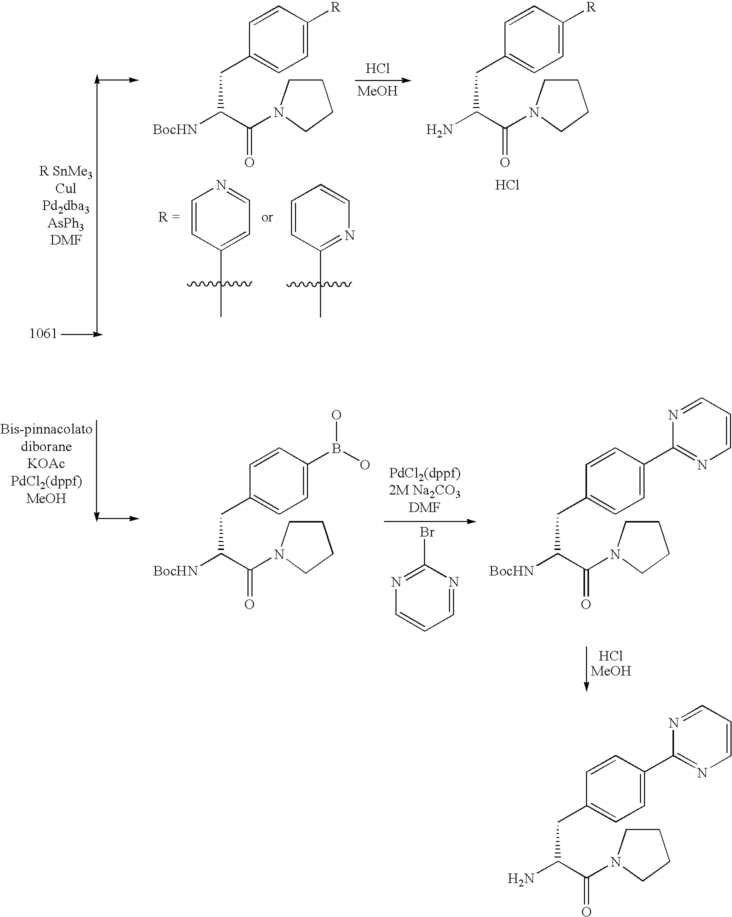

### General Procedure J

### GENERAL PROCEDURE FOR FORMING A 2- OR 4-PYRIDYL SUBSTITUENT ON A PHENYL GROUP

### Exemplified by the Preparation of 1-[(R)-1-Pyrrolidin-1-ylcarbonyl-1-amino-2-(4-(2-or 4pyridyl)phenyl]ethane

1-(R)-[1-(t-butoxycarbonylamino)-1-(pyrrolidin-1-ylcarbonyl)-2-(4-iodophenyl)]ethane (compound 1061) (300 mg, 0.68 mmol), is added to a 50 mL round-bottom flask with CuI (8% mol) in dry DMF (10 mL). The resulting solution is flushed under nitrogen for 2-3 min. Pd₂dba₃ (2% mol) (Aldrich) and AsPh₃ (16% mol) (Aldrich) are weighed together in a small vial to which 1 mL of DMF is added. This solution is added to the reaction mixture and it is flushed under nitrogen for an additional 2-3 minutes. An oil bath is heated to 60° C. and the reaction mixture is immersed into it and allowed to thermally equilibrate. The commercially available pyridyl stannane (1.15 eq.) (Frontier) is then weighed out into a small vial to which 1 mL of DMF is added and this solution is then added to the previous reaction mixture and heated at 60° C. for 6 hours. The solvent is removed under vacuum. The crude residue is dissolved in EtOAc (30 mL). The organic layer is washed with brine (10 mL, 2×), and dried over MgSO₄. Upon filtration and evaporation of the solvent under reduced pressure, the crude material is purified on column chromatography (silica gel), eluted with EtOAc-Hexanes 3:2 to afford 1-[(R)-1-(pyrrolidin-1-ylcarbonyl)-1-(t-butoxycarbonylamino)-2-(4-(2-or 4-pyridyl)phenyl]ethane in good yield.

Subsequent removal of the Boc protecting group with HCl/methanol in the manner described above provides for the title compound as the HCl salt.

### General Procedure K

### GENERAL PROCEDURE FOR FORMING A 2-PYRIMIDINYL SUBSTITUENT ON A PHENYL GROUP

### Exemplified by the Preparation of 1-[(R)-1-Pyrrolidin-1-ylcarbonyl-1-amino-2-(4-(2-pyrimidinyl)phenyl]ethane

1-(R)-[1-(t-butoxycarbonylamino)-1-(pyrrolidin-1-ylcarbonyl)-2-(4-iodophenyl)]ethane (compound 1061)(100 mg, 0.22 mmol), is dissolved in dry MeOH (5 mL) to which is added KOAc (1.5 eq.) and bis-pinnacolato diboron (1.1 eq.) (Aldrich) and the mixture is flushed under nitrogen for 5 minutes. The catalyst, PdCl₂(dppf) (0.03 eq.) (Aldrich), is then added and the reaction is heated at 60° C. overnight. The reaction mixture is filtered through Celite and condensed under vacuum. The residue is then treated with bromopyrimidine (3 eq.) (Aldrich), Na₂CO₃ (5 eq., 0.55 mL) and PdCl₂(dppf) (0.03 eq.) in DMF (1 mL) and is stirred at 80° C. overnight. The solvent is removed under vacuum. The crude residue is purified on column chromatography (silica gel), eluted with EtOAc-Hexanes, 3:2 to afford 1-[(R)-1-pyrrolidin-1-ylcarbonyl-1-(t-butoxycarbonylamino)-2-(4-(2-pyrimidinyl)phenyl]ethane in good yield.

Subsequent removal of the Boc protecting group with HCl/methanol in the manner described above provides for the title compound as the HCl salt.

The processes set forth in General Procedure L below are illustrated in the following reaction scheme:

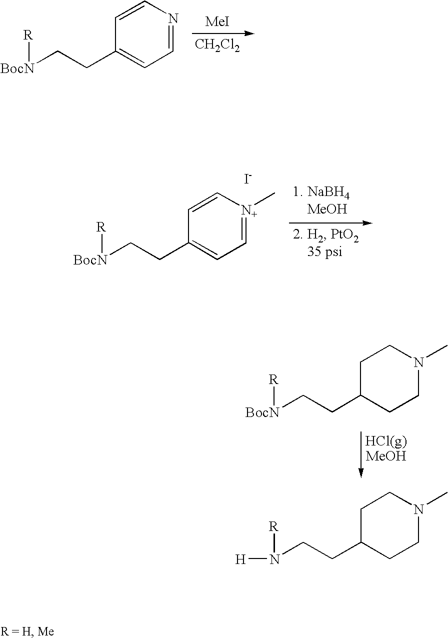

### General Procedure L

### GENERAL PROCEDURE FOR THE PREPARATION OF 1,2,3,6-TETRAHYDRO-N-(ALKYL)PYRIDINE DERIVATIVES

Boc protected 2-aminoethylpyridine (or the N-methyl analog thereof) (120 mg, 0.18 mmol), is dissolved in MeOH/CH₂Cl₂ (2:1) to make a 2.5 M solution. To this is added MeI (4 eq.) and the mixture is heated in a sealed tube for 3.5 h. The solvent is removed under vacuum and the resulting crude mixture can be used directly without purification and/or isolation.

### General Procedure M

### GENERAL PROCEDURE FOR THE REDUCTION/HYDROGENATION OF A PYRIDINIUM SALT

The methyl pyridinium iodide salt produced above, (60 mg, 0.083 mmol), is dissolved in dry MeOH (4 mL) and the resulting mixture cooled to 0° C. Excess NaBH₄ was added and the mixture is allowed to stir for 30 min. The solvent is then removed under vacuum and water (5-10 mL) is added to the crude product and sonicated for 10 min. Upon filtration, the solvent is evaporated to provide for Boc protected 2-aminoethyl-1,2,3,6-tetrahydro-pyridine in good yields.

If desired, the remaining unsaturated bond in the Boc protected 2-aminoethyl-1,2,3,6-tetrahydropyridine can be hydrogenated with hydrogen/PtO₂ maintained at about 35 psi.

The Boc protecting group of the saturated or unsaturated compound can then be removed by conventional methods (e.g., HCl/methanol).

The processes set forth in General Procedure N below are illustrated in the following reaction scheme:

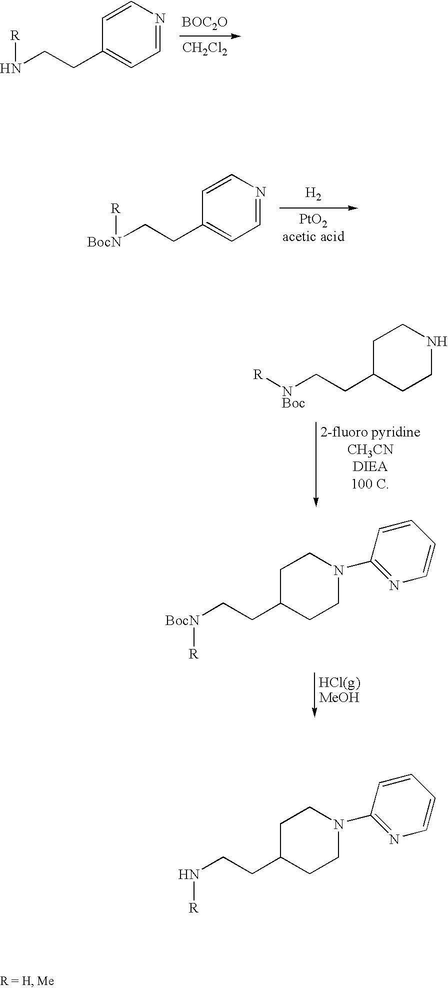

### General Procedure N

### GENERAL PROCEDURE FOR PREPARING N-(PYRID-2-YL)PIPERIDINE COMPOUNDS

### Exemplified by the Preparation of 2-[1-(pyrid-2-yl)piperidin-4-yl]ethylamine

### Step A: Synthesis of N-t-butoxycarbonyl 2-(pyrid-2-yl)ethylamine

4-Aminoethylpyridine (5.0 g, 40 mmol) and di-t-butyl dicarbonate (8.9 g, 40 mmol) are dissolved in CH₂Cl₂ (50 mL) and the resulting solution is stirred at rt for overnight. Solvent is removed under reduced pressure to afford N-t-butoxycarbonyl 2-(pyrid-2-yl) ethylamine as a reddish liquid (9.1 g, 100%).

### Step B: Synthesis of N-t-butoxycarbonyl 2-(piperidin-2-yl)ethylamine

The product from step A is mixed with PtO₂ (640 mg) in HOAc (30 mL) and hydrogenation is carried out at 58 psi on a Parr apparatus overnight. Catalyst is removed and solvent is evaporated under reduced pressure to give N-t-butoxycarbonyl 2-(piperidin-2-yl)ethylamine as a black liquid.

### Step C: Synthesis of N-t-butoxycarbonyl 2-[1-(pyrid-2-yl)piperidin-4-yl]ethylamine

To a solution of N-t-butoxycarbonyl 2-(piperidin-2-yl)ethylamine (8.1 g) and DIEA (14.1 mL) in CH₃CN (29 mL) is added 2-fluoropyridine (3.5 mL) and the resulting mixture is heated in a sealed-tube at 100° C. for three days. Solvent is removed and the crude product is purified via column chromatography (20% EtOAc/hexane) to afford 3.9 g of N-t-butoxycarbonyl 2-[1-(pyrid-2-yl)piperidin-4-yl]ethylamine. ¹H NMR (CDCl₃)=8.16 (dd, J=1.8, 5.0 Hz, 1H), 7.44-7.38 (m, 1H), 6.61 (d, J=8.7 Hz, 1H), 6.53 (dd, J=5.0, 7.2, 1H), 4.58 (bs, 1H), 4.23 (d, J=12.6 Hz, 2H), 3.15 (q, J=6.6 Hz, 2H), 2.76 (dt, J=2.7, 12.6 Hz, 2H), 1.75 (d, J=12.6 Hz, 2H), 1.55-1.35 (m, 11H), 1.28-1.15 (m, 3H);

MS: m/z (EI⁺) 306 (M⁺+H);

HPLC (CH₃CN—H₂O-0.1%TFA) (short column) Rₜ=2.27 min.

### Step D: Synthesis of 2-[1-(pyrid-2-yl)piperidin-4-yl]ethylamine

To a solution of N-t-butoxycarbonyl 2-[1-(pyrid-2-yl)piperidin-4-yl]ethylamine (3.9 g) in EtOAc (15 mL) was bubbled HCl (g) for 15 min. The suspension was then stirred under positive pressure (N₂) for 30 min. Solvent was removed under vacuum to afford the 2-[1-(pyrid-2-yl)piperidin-4-yl]ethylamine (pure) as the hydrochloride salt (white solid) (3.4 g, 98%).

The processes set forth in General Procedure O below are illustrated in the following reaction scheme:

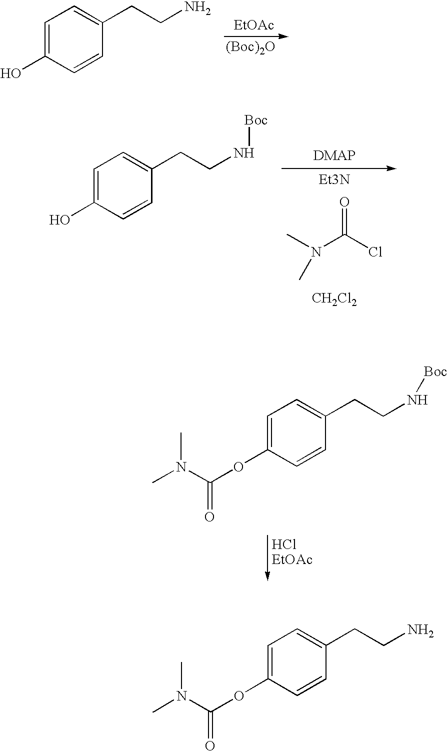

### General Procedure O

### GENERAL PROCEDURE FOR THE PREPARATION OF CARBAMOYLOXY SUBSTITUTED PHENYLETHYL AMINE COMPOUNDS

### Exemplified by the Preparation of 2-[4-(N′,N′-dimethylaminocarbonyloxy)phenyl]ethylamine

### Step A: Synthesis of N-t-butoxycarbonyl 2-(4-hydroxyphenyl)ethylamine

The amine group of 2-(4-hydroxyphenyl)ethylamine can be protected with a Boc protecting group in the manner described above to provide for N-t-butoxycarbonyl 2-(4-hydroxyphenyl)ethylamine.

### Step B: Synthesis of N-t-butoxycarbonyl 2-[4-(N′,N′-dimethylaminocarbonyloxy)phenyl]ethylamine

N-t-butoxycarbonyl 2-(4-hydroxyphenyl)ethylamine (2.53 g, 10.7 mmol), Et₃N (2.96 mL, 2 eq.), a catalytic amount of DMAP (131 mg) and dimethylcarbamyl chloride (2.0 mL, 2 eq) are mixed in CH₂Cl₂ at 0° C. The resulting mixture is stirred overnight. EtOAc is added to dilute the reaction mixture and then is washed with 1N HCl, sat.Na₂CO₃ and brine. Solvent is removed under reduced pressure to give pure t-butoxycarbonyl 2-[4-(N′,N′-dimethylaminocarbonyloxy)phenyl]ethylamine as a colorless solid.

### Step C: Synthesis of 2-[4-(N′,N′-dimethylaminocarbonyl-oxy)phenyl]ethylamine

The Boc protecting group on the t-Butoxycarbonyl 2-[4-(N′,N′-dimethylaminocarbonyloxy)phenyl]ethylamine is removed in a manner described above to provide for the title compound as a white solid, and this compound is used “as is” in the next step.

The processes set forth in General Procedure P below are illustrated in the following reaction scheme:

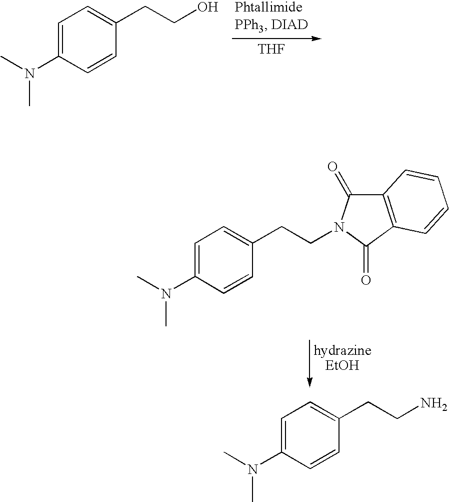

### General Procedure P

### GENERAL PROCEDURE FOR CONVERTING 2-[4-(N,N-DIMETHYLAMINOPHENYL]ETHANOL TO 2-[4-(N′,N′-DIMETHYLAMINOPHENYL]ETHYLAMINE

### Step A: Synthesis of 2-[2-(4-N,N-dimethylaminophenyl)-ethyl]-isoindole-1,3-dione

2-[4-(N,N-dimethylaminophenyl]ethanol (2.05 g, 17.4 mmol), phthalimide (2.19 g, 14.9 mmol) and PPh₃ (3.93 g, 14.9 mmol) (Aldrich) are mixed in 100 mL of THF maintained at 0° C. The mixture is then treated with DIAD (2.68 mL) (Aldrich) which was added dropwise. After stirring overnight, the solvent is removed under reduced pressure to give a pale yellow solid. The solid is triturated with EtOAc three times. The combined EtOAc layers are treated with gaseous HCl to precipitate the product, and the desired product is isolated through filtration.

### Step B: Synthesis of 2-[4-(N′,N′-dimethylaminophenyl]ethylamine

2-[2-(4-N,N-dimethylaminophenyl)-ethyl)-isoindole-1,3-dione (606 mg, 1.84 mmol) and hydrazine hydrate (30%, 0.64 mL) in ethanol is heated at 65° C. for 5 h. The precipitate is removed via filtration. The filtrate is concentrated to give the title compound as a white solid. This product is used in the next step without further purification.

The processes set forth in General Procedure Q below are illustrated in the following reaction scheme:

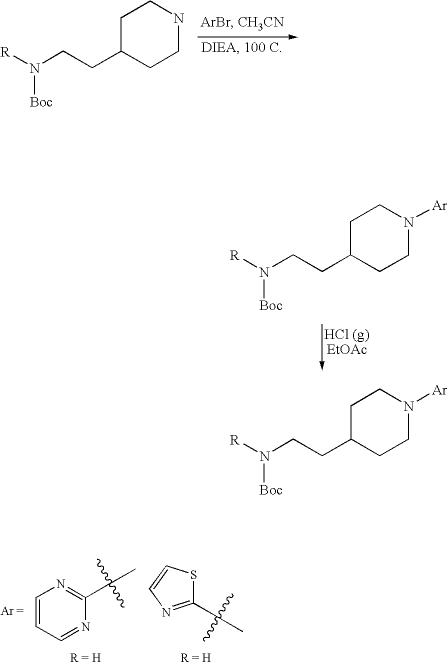

### General Procedure Q

### GENERAL PROCEDURE FOR PREPARING 2-[(1-PYRIMIDIN-2-YL)PIPERIDIN-4-YL]-ETHYLAMINE

### Step A: Synthesis of N-t-butoxycarbonyl 2-[1-(pyrimidin-2-yl)piperidin-4-yl]-ethylamine

N-t-butoxycarbonyl 2-(piperidin-4-yl)-ethylamine (as described above), DIEA (0.75 mL) and 2-bromopyrimidine (204 mg) (Aldrich) in acetonitrile (5 mL) are heated under reflux overnight. The solvent is removed under reduced pressure and the black liquid is subjected to a column chromatography, eluted with 1:1 EtOAc/hexanes, to give pure N-t-butoxy-carbonyloxy 2-[1-(pyrimidin-2-yl)piperidin-4-yl]-ethylamine as a pale yellow oil. ¹H NMR (CDCl₃)=8.21 (d, J=5.1 Hz, 2H), 6.36 (t, J=5.1 Hz, 1H), 4.64 (d, J=13.8 Hz, 2H), 3.14-3.07 (m, 2H), 2.76 (dt, J=2.7, 13.2 Hz, 1H), 1.69 (d, J=13.8 Hz, 1H), 1.57-1.30 (m, 11H), 1.20-1.03 (m, 3H);

MS: m/z (EI⁺) 307 (M⁺+H);

HPLC (CH₃CN—H₂O-0.1% TFA) (short column) Rₜ=2.63 min.

### Step B: Synthesis of 2-[(1-pyrimidin-2-yl)piperidin-4-yl]-ethylamine

The Boc protecting group on N-t-butoxy-carbonyl 2-[1-(pyrimidin-2-yl)piperidin-4yl]-ethylamine is removed as described above to afford the title compound.

The processes set forth in General Procedure R below are illustrated in the following reaction scheme:

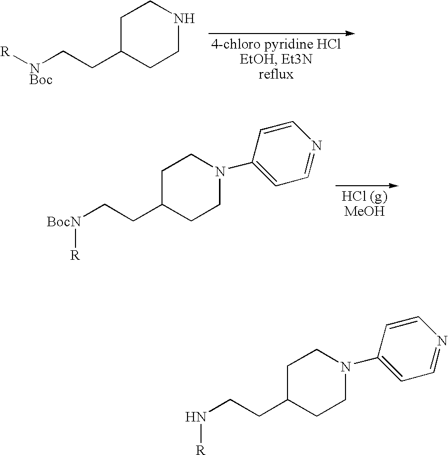

### General Procedure R

### GENERAL PROCEDURE FOR PREPARING N-(PYRID-4-YL)PIPERIDINE COMPOUNDS

### Step A: Synthesis of N-t-butoxycarbonyl 2-[1-(pyrid-4-yl)piperidin-4-yl]-ethylamine

N-t-butoxycarbonyl 2-(piperidin-4-yl)-ethylamine (prepared as above) (14.4 g, 50 mmol), 4-chloropyridine HCl (1.0 eq., 8.0 g), TEA (2.2 eq.) are mixed in ethanol, and maintained under reflux overnight. The desired compound, N-t-butoxycarbonyl 2-[1-(pyrid-4-yl)piperidin-4-yl]-ethylamine, is isolated by column chromatography, (silica gel) eluted with EtOAc and carried to the next step.

### Step B: Synthesis of 2-[1-(pyrid-4-yl)piperidin-4-yl]-ethylamine

The Boc protecting group on N-t-butoxycarbonyl 2-[1-(pyrid-4-yl)piperidinyl-4-yl]-ethylamine is then removed using procedures described above to provide the title compound.

The process set forth in General Procedure S below is illustrated in the following reaction scheme:

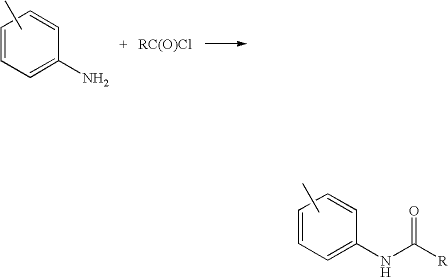

### General Procedure S

To a solution of the starting aniline (100 mg; 0.19 mmol) in dry pyridine (5 mL), is added acetic anhydride (20 μL). The mixture is stirred at rt overnight. Water (3 mL) is added to the mixture and the product was precipitated from the solution.

The following Examples illustrate the synthesis of certain intermediates and compounds of Formula I of this invention.

The compounds shown in Table I (compounds 1-21) and related compounds are made using the procedures described in the following examples.

### Example 1

### Preparation of 3[2′-{(4″-chloro-2″,5″-dimethylbenzene)-N′-methylsulfonamido)-phenyl]-N-[1″-R-1″′-(N″-pyrrolidinylcarbonyl)-2-(4-pyridyl)eth-1-yl]propionamide (1)

### Step a) Preparation of 3-(2-amino phenyl) sodium propionate

A suspension of 2-nitrocinnamic acid, (10 g, 0.05 mol, 1 eq), a catalytic amount of 10% Pd/C, NaOH (2.07 g, 0.05 mol, 1 eq), and H₂O (250 ml), were shaken on a Parr apparatus at 40 PSI for 3 hours, at room temperature. The reaction mixture was filtered through celite and evaporated under vacuum to give the title compound.

¹H NMR (DMSO-d₆) δ 6.84 (m, 2H), 6.53 (d, 1H), 6.43 (t, 1H), 5.04 (s, 2H) 2.60 (t, 2H), 2.17 (t, 2H).

### Step b) Preparation of 3-[2-(4-Chloro-2,5-dimethyl-benzenesulfon-amido)-phenyl]-propionic acid

To a solution of sodium 3-(2-amino-phenyl)-propionate (2 g, 10.64 mmol, 1 eq), in 1N NaOH (10.64 ml, 10.64 mmol, 1 eq) and H₂O (10 ml) at 0° C. was added dropwise 4-Chloro-2,5-dimethyl-benzenesulfonyl chloride (2.54 g, 10.64 mmol, 1 eq) in THF (15 ml). The mixture was stirred at this temperature for 1 hour. The organic layer was evaporated under vacuum. The aqueous mixture was acidified with 1N HCl and extracted with EtOAc (2×40 ml). The organic layers were dried over Na₂ SO₄ and the solvent removed under vacuum to give the title compound.

HPLC (CH₃CN—H₂O-0.1% TFA) (short column): Rₜ=4.38 min.

### Step c) Preparation of 3-methyl-[N-methyl-2-(4-Chloro-2,5-dimethyl-benzenesulfonamido)-phenyl]-propionate

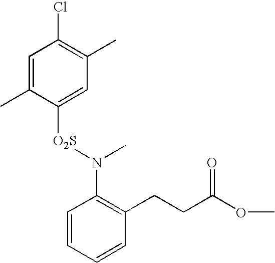

To a solution of 3-[2-(4-chloro-2,5-dimethylbenzenesulfonamido)-phenyl]-propionic acid (2.56 g crude, 6.97 mmol, 1 eq) in CH₂Cl₂: MeOH (3:1) was added dropwise trimethylsilyl diazomethane, 2.0 M solution in hexanes, (17.4 ml, 5 eq) at room temperature. The mixture was stirred overnight and quenched with acetic acid. The solvent was removed under vacuum and the crude material purified by column chromatography over silica gel with EtOAc:Hexanes (5:95) as eluent to give the title compound

HPLC (CH₃CN—H₂O-0.1% TFA) (short column): Rₜ=5.65 min.

### Step d) Preparation of 3-[N-methyl-2-(4-chloro-2,5-dimethyl-benzenesulfonamido)-phenyl]-propionic acid

3-methyl-[N-methyl-2-(4-chloro-2,5-dimethylbenzenesulfonylamino)-phenyl]-propionate was hydrolyzed using LiOH (1.0 eq) in MeOH:H₂O (1:1), at room temperature for 5 hours. The reaction mixture was condensed under vacuum and the remaining aqueous mixture cooled down via ice bath and acidified with 1N HCl, to pH 1. The resultant precipitate was isolated via filtration to give the title compound as a white solid.

¹H NMR (DMSO-d₆) δ 12.15 (s, 1H), 7.69 (s, 1H), 7.49 (s, 1H), 7.35 (d, 1H), 7.28 (t, 1H), 7.11 (t, 1H), 6.67 (d, 1H), 3.08 (m, 4H), 2.74 (m, 1H), 2.34 (s, 3H), 2.08 (s, 3H).

### Step e) Preparation of 3[2′-{(4″-chloro-2″,5″-dimethylbenzene)-N′-methylsulfonamido)-phenyl]-N-[1″′-R-1″′-(N″-pyrrolidinylcarbonyl)-2-(4-pyridyl)eth-1-yl]propionamide

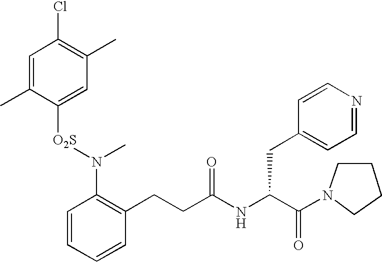

3-[N-methyl-2-(4-Chloro-2,5-dimethyl-benzenesulfonamido)-phenyl]-propionic acid (1.0 eq) and 1-(R)-[1-amino-1-(pyrrolidin-1-ylcarbonyl)-2-(4-pyridyl)]ethane (1.0 eq) were stirred at room temperature in CH₃CN. To this was added, Et₃N (3.0 eq), and after a few minutes HATU (1.0 eq). The reaction mixture was stirred overnight. EtOAc was added. The organic layer was washed with a saturated solution of NaHCO₃ and brine, dried over MgSO₄. Upon filtration, and evaporation of the solvent under reduced pressure, the crude material was purified by column chromatography (silica gel) with EtOAc, to give the title material.

HPLC (CH₃CN—H₂O-0.1% TFA) (short column): Rₜ=3.34 min.

### Example 2

### Preparation of 3[2′-{(4″-chloro-2″,5″-dimethylbenzene)-N′-methylsulfonamido)-phenyl]-N-[2-(N″-methylpiperidine-4-yl)eth-1-yl]propionamide (2)

The title compound was prepared using the procedures outlined in Example 1, substituting 2-(N(methyl)piperidin-4-yl)ethyl amine in Step e), as a TFA salt. The desired material was purified by reverse phase HPLC and isolated as a TFA salt.

HPLC (CH₃CN/water-0.1% TFA) (short column): Rₜ=3.31 min.

### Example 3

### Preparation of 3[2′-{(4″-chloro-2″,5″-dimethylbenzene)-N′-methylsulfonamido)-phenyl]-N-[1-R-1-(N″-piperidinylcarbonyl]eth-1-yl]]propionamide (3)

### Step a) Preparation of 2-R-amino 1-(R)-[1-amino-1-(pyrrolidin-1-ylcarbonyl)]ethane

2-R-tert-Butoxycarbonylaminopropionic acid was reacted with piperidine (1.0 eq) using EDCI (1.0 eq), HOBT (1.0 eq),Et₃N (3.0 eq), at ice bath temperature in CH₂Cl₂. The organic layer was washed with brine, NaHCO₃ saturated and 1N HCl. The organic layer was dried over MgSO₄. Upon evaporation of the solvent under reduced pressure, the desired material was isolated as a foam. The Boc-amine was deprotected using HCl in EtOAc, for 2 hours at room temperature. The HCl salt was obtained in quantitative yields.

### Step b) Preparation of 3[2′-{(4″-chloro-2″,5″-dimethylbenzene)-N′-methylsulfonamido)-phenyl]-N-[1-R-1-(N″-piperidinylcarbonyl]eth-1-yl]]propionamide

The title compound was prepared using the procedures outlined in Example 1, Step e), substituting the above amine prepared in Step a) above as an HCl salt. The desired material was then purified by column chromatography over silica gel with EtOAc:Hexanes:NH₄OH (50:50:1) as eluent to give the title material.

HPLC (CH₃CN—H₂O-0.1% TFA) (short column): Rₜ=4.88 min.

### Example 4

### Preparation of 3[2′-{(4″-chloro-2″,5″-dimethylbenzene)-N′-methylsulfonamido)-phenyl]-N-[1-S-1-(N″-piperidinylcarbonyl]eth-1-yl]]propionamide (4)

The title compound was prepared using the procedures outlined in Example 3, substituting 2-R-tert-butoxycarbonylamino-propionic acid with 2-S-tert-butoxy-carbonylaminopropionic acid in Step a).

The crude material was then purified by column chromatography over silica gel with EtOAc:Hexanes:NH₄OH (50:50:1) as eluent to give the title material.

HPLC (CH₃CN—H₂O-0.1% TFA) (short column): Rₜ=4.87 min.

### Example 5

### Preparation of 3[2′-{(4″-chloro-2″,5″-dimethylbenzene)-N′-methylsulfonamido)-phenyl]-N-[2-{N″-(4-methylpyrid-2-yl)}piperidin-4-yl]eth-1-yl]propionamide (5)

The title compound was prepared using the procedure outlined in Example 1, substituting with 2-(N-(4-methylpyrid-2-yl)piperidin-4-yl)ethylamine in step e) as a TFA salt. The crude material was then purified by preparatory chromatography over silica gel with 8% MeOH/CH₂Cl₂+NH₄OH as eluent to give the title material.

HPLC (CH₃CN—H₂O-0.1% TFA) (short column): Rₜ=3.72 min.

### Example 6

### Preparation of 3[2′-{(4″-chloro-2″,5″-dimethylbenzene)-N′-methylsulfonamido)-phenyl]-N-[1-R-1-(N″-pyrrolidinylcarbonyl)-2-(4-pyridylphen-4-yl)eth-1-yl]propionamide (6)

The title compound was prepared using the procedure outlined in Example 1, substituting with 1-[R-1-pyrrolidin-1-ylcarbonyl-1-amino-2-(4-pyridyl)phenyl]ethane as an HCl salt in step e). The crude material was then purified by reverse phase HPLC (acetonitrile/water-0.1% TFA) and isolated as a TFA salt to afford the title material.

HPLC (CH₃CN—H₂O-0.1% TFA) (short column): Rₜ=3.50 min.

### Example 7

### Preparation of 3[2′-{(4″-chloro-2″,5″-dimethylbenzene)-N′-methylsulfonamido)-phenyl]-N-[(2-N″-piperidinyl)eth-1-yl]propionamide (7)

The title compound was prepared using the procedure outlined in Example 1, using 2-piperidin-1-yl-ethylamine. The crude material was then purified by reverse phase HPLC (acetonitrile/water-0.1% TFA) and isolated as a TFA salt to afford the title material.

HPLC (CH₃CN—H₂O-0.1% TFA) (short column): Rₜ=3.46 min.

### Example 8

### Preparation of 3[2′-{(4″-chloro-2″,5″-dimethylbenzene)-N′-methylsulfonamido)-phenyl]-N-[(2-pyrid-4-yl)eth-1-yl]propionamide (8)

The title compound was prepared using the procedure outlined in Example 1, and 2-ethyl amino pyridine. The crude material was then purified by reverse phase HPLC (acetonitrile/water-0.1% TFA) and isolated as a TFA salt to afford the title material.

HPLC (CH₃CN—H₂O-0.1% TFA) (short column): Rₜ=3.27 min.

### Example 9

### Preparation of 3[2′-{(4″-chloro-2″,5″-dimethylbenzene)-N′-methylsulfonamido)-phenyl]-N-[(2-(N″-{(2-pyridyl)piperidin-4-yl}eth-1-yl]propionamide (9)

The title compound was prepared using the procedure outlined in Example 1, substituting 2-[1-(pyrid-2-yl)piperidin-4-yl]ethylamine, as a TFA salt in Step e). The crude material was then purified by reverse phase HPLC (acetonitrile/water-0.1% TFA) and isolated as a TFA salt to afford the title material.

HPLC (CH₃CN—H₂O-0.1% TFA) (short column): Rₜ=3.64 min.

### Example 10

### Preparation of 3[2′-{(4″-chloro-2″,5″-dimethylbenzene)-N′-methylsulfonamido)-phenyl]-N-[(2-(N″-ethylpiperidin-4-yl)eth-1-yl]propionamide (10)

### Step a) Preparation of 2-(N-ethyl piperidin-4-yl)ethylamine

The title compound was prepared from 2-aminoethyl pyridine and ethyl iodide using General Procedures L and M. The desired material was isolated as a TFA salt.

### Step b) Preparation of 3[2′-{(4″-chloro-2″,5″-dimethylbenzene)-N′-methylsulfonamido)-phenyl]-N-[-[(2-(N″-ethylpiperidin-4-yl)eth-1-yl]propionamide

The title compound was prepared using the procedure outlined in Example 1, and the amine prepared in Steb a) above. The crude material was then purified by reverse phase HPLC (acetonitrile/water-0.1% TFA) and isolated as a TFA salt to afford the title material.

HPLC (CH₃CN—H₂O-0.1% TFA) (short column): Rₜ=3.36 min.

### Example 11

### Preparation of 3[2′-{(4″-chloro-2″,5″-dimethylbenzene)-N′-methylsulfonamido)-phenyl]-N-[1-S-1-methyl-2-(N″-piperidinyl)eth-1-yl]propionamide (11)

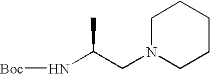

### Step a) Preparation of 2-S-[(tert-butoxycarbonyl)amino]-1-piperin-1-yl-propane

To a suspension of NaBH₄ (71 mg, 1.87 mmol, 2.4 eq) in THF (0.2 M) at 0° C. was added dropwise (1-S-Methyl-2-oxo-2-piperidin-1-yl-ethyl)-carbamic acid tert-butyl ester (200 mg, 0.78 mmol, 1 eq) in THF (0.2 M). The mixture stirred at 0° C. for 20 minutes at which point a solution of iodine (198 mg, 0.78 mmol, 1 eq) in THF (0.2 M) was added dropwise and the mixture stirred from 0° C. to room temperature overnight. The mixture was cooled via ice bath, quenched dropwise with MeOH and the solvent was removed under vacuum. The residue was dissolved in 20% NaOH and extracted with CH₂Cl₂ (2×20 ml) and CHCl₃:IPA (90:10, 1×20 ml). The organic layers were combined and dried over Na₂SO₄ and the solvent removed under vacuum to give the desired material which was used in the next step without purification.

### Step b) Preparation of 2-S-amino-1-piperin-1-yl-propane

The -boc compound in Step a) was dissolved in dichloromethane and the Boc group was removed with an excess TFA. The reaction mixture was stirred at room temperature for a few hours. The solvent was evaporated under reduced pressure, and the desired material was isolated as a di-TFA salt.

### Step c) Preparation of 3[2′-{(4″-chloro-2″,5″-dimethylbenzene)-N′-methylsulfonamido)-phenyl]-N-[1-S-1-methyl-2-{N″-piperidinyl)eth-1-yl]propionamide

The title compound was prepared using the procedure outlined in Example 1, using the above amine, as a TFA salt, in Step e). The crude material was then purified by reverse phase HPLC (acetonitrile/water-0.1% TFA) and isolated as a TFA salt to afford the title material.

HPLC (CH₃CN—H₂O-0.1% TFA) (short column): R=3.61 min

### Example 12

### Preparation of 3[2′-{(4″-chloro-2″,5″-dimethylbenzene)-N′-methylsulfonamido)-phenyl]-N-[-2-(N″-(pyrid-4-yl}piperidin-4-yl)eth-1-yl]propionamide (12)

The title compound was prepared using the procedure outlined in Example 1, using 2-[1-(pyrid-4-yl)piperidin-4-yl]ethylamine, as a TFA salt, in Step e). The crude material was then purified by column chromatography over silica gel with 1-3% MeOH/CH₂Cl₂+NH₄OH as eluent to afford the title material.

HPLC (CH₃CN—H₂O-0.1% TFA) (short column): Rₜ=3.56 min.

### Example 13

### Preparation of 3[2′-{(4″-chloro-2″,5″-dimethylbenzene)-N′-methylsulfonamido)-phenyl]-N-[1-R-1-methyl-2-(N″-piperidinyl)eth-1-yl]propionamide (13)

The title material was prepared in the same manner as Example 11, starting with (1-R-Methyl-2-oxo-2-piperidin-1-yl-ethyl)-carbamic acid tert-butyl ester. The crude material was then purified by reverse phase HPLC (acetonitrile/water-0.1% TFA) and isolated as a TFA salt to afford the title compound.

HPLC (CH₃CN—H₂O-0.1% TFA) (short column): Rₜ=3.59 min.

### Example 14

### Preparation of 3[2′-{(4″-chloro-2″,5″-dimethylbenzene)-N′-methylsulfonamido)-phenyl]-N-[2-{N″-(3-methylpyrid-2-yl}piperidin-4-yl)eth-1-yl]propionamide (14)

The title material was prepared using the procedure outlined in Example 1, substituting 2-[1-(3-methylpyrid-2-yl)piperidin-4-yl]ethylamine in Step e) as a TFA salt. The crude material was then purified by column chromatography over silica gel with 1% MeOH/CH₂Cl₂+NH₄OH as eluent to give the title material.

HPLC (CH₃CN—H₂O-0.1% TFA) (short column): Rₜ=3.70 min.

### Example 15

### Preparation of 3[2′-{(4″-chloro-2″,5″-dimethylbenzene)-N′-methylsulfon-amido)-phenyl]-N-[1-S-1-methyl-2-(4-methylpiperazin-1-yl)eth-1-yl]propionamide (15)

### Step a) Preparation of 1-oxo-2-S-[(tert-butoxycarbonyl)amino]-1-(4-methyl-piperazin-1-yl)propane

2-S-tert-Butoxycarbonylamino-propionic acid was reacted with N-methyl piperazine (1.0 eq) using EDCI (1.0 eq), HOBT (1.0 eq),Et₃N (3.0 eq), at ice bath temperature in CH₂Cl ₂. The organic layer was washed with brine, NaHCO₃ saturated. The organic layer was dried over MgSO₄. Upon evaporation of the solvent under reduced pressure, the desired material was isolated as a foam.

### Step b) Preparation of 2-S-[(tert-butoxycarbonyl)amino]-1-(4-methyl-piperazin-1-yl)propane

To a suspension of NaBH₄ (71 mg, 1.87 mmol, 2.4 eq) in THF (0.2 M) at 0° C. was added dropwise (1-S-Methyl-2-oxo-2-(4-methyl piperazine)-1-yl-ethyl)-carbamic acid tert-butyl ester (200 mg, 0.78 mmol, 1 eq) in THF (0.2 M). The mixture stirred at 0° C. for 20 minutes at which point a solution of iodine (198 mg, 0.78 mmol, 1 eq) in THF (0.2 M) was added dropwise and the mixture stirred from 0° C. to room temperature overnight. The mixture was cooled via ice bath, quenched dropwise with MeOH and the solvent was removed under vacuum. The residue was dissolved in 20% NaOH and extracted with CH₂Cl₂ (2×20 ml) and CHCl₃:IPA (90:10, 1×20 ml). The organic layers were combined and dried over Na₂ SO₄ and the solvent removed under vacuum to give the desired material which was used in the next step without purification.

### Step c) Preparation of 1-S-methyl-2-(4-methyl-piperazin-1-yl)-ethylamine

The Boc amine from Step b) was deprotected using TFA in CH₂Cl₂, at room temperature for 2 hours. The solvent was evaporated under reduced pressure to give the title amine as a di-TFA salt.

### Step d) Preparation of 3[2′-{(4″-chloro-2″,5″-dimethylbenzene)-N′-methylsulfonamido)-phenyl]-N-[1-S-1-methyl-2-(4-methylpiperazin-1-yl)eth-1-yl]propionamide

The title material was prepared using the procedure outlined in Example 1, substituting 1-S-methyl-2-(4-methyl-piperazin-1-yl)-ethylamine in Step e) as a TFA salt. The crude material was then purified by reverse phase HPLC (acetonitrile/water-0.1% TFA) to give the title material.

HPLC (CH₃CN—H₂O-0.1% TFA) (short column): Rₜ=2.98 min

### Example 16

### Preparation of 3[2′-{(4″-chloro-2″,5″-dimethylbenzene)-N′-methylsulfon-amido)-phenyl]-N-[1-R-1-methyl-2-(4-methylpiperazin-1-yl)eth-1yl]propionamide (16)

The title material was prepared using the procedure outlined in Example 15, substituting with 2-R-tert-butoxy carbonylamino propionic acid. The crude material was then purified by reverse phase HPLC (acetonitrile/water-0.1% TFA) and isolated as a TFA salt to afford the desired material.

HPLC (CH₃CN—H₂O-0.1% TFA) (short column): Rₜ=3.00 min.

### Example 17

### Preparation of 3[2′-{(4″-chloro-2″,5″-dimethylbenzene)-N′-methylsulfonamido)-phenyl]-N-[methyl-N-2-(N″-methylpiperidin-4-yl)eth-1-yl]propionamide (17)

The title material was prepared using the procedure outlined in Example 1, substituting N-methyl-2-(N(methyl)piperidin-4-yl)ethyl amine in Step e) as a TFA salt. The crude material was purified by reverse phase HPLC (acetonitrile/water-0.1% TFA) to give the desired material

MS(ES) m/e 522 (M+H).

### Example 18

### Preparation of 3[2′-{(2″,3″-dichlorobenzene)-N′-methylsulfonamido)-phenyl]-N-[methyl-N-2-(N″-methylpiperidin-4-yl)eth-1-yl]propionamide (18)

The title material was prepared using the procedure outlined in Example 1, substituting 2,3-dichlorobenzene sulfonyl chloride in Step b), and N-methyl-2-(N(methyl)piperidin-4-yl)ethyl amine in Step e) as a TFA salt. The crude material was purified by reverse phase HPLC (acetonitrile/water-0.1% TFA) to give the desired material.

MS (ES) m/e 527 (M+H).

### Example 19

### Preparation of 3[2′-{(4″-chloro-2″,5″-dimethylbenzene)-N′-methylsulfonamido)-phenyl]-N-[(α-(R,S)-methoxycarbonyl)benzyl]propionamide (19)

The title material was prepared using the procedure outlined in Example 1, substituting α-(R,S)-methoxycarbonyl benzylamino in Step e) as an HCl salt. The crude material was purified by reverse phase HPLC (acetonitrile/water-0.1% TFA) to give the desired material.

MS (ES) m/e 530 (M+H)

### Example 20

### Preparation of 3[2′-{(2″,3″-dichlorobenzene)-N′-methylsulfonamido)-phenyl]-N-[(α-(R,S)-methoxycarbonyl)benzyl]propionamide (20)

The title material was prepared using the procedure outlined in Example 1, substituting 2,3-dichlorobenzene sulfonyl chloride in Step b) and α-(R,S)-methoxycarbonyl benzylamino in Step e) as an HCl salt. The crude material was purified by reverse phase HPLC (acetonitrile/water-0.1% TFA) to give the desired material.

MS (ES) m/e 536 (M+H)

### Example 21

### Preparation of 3[2′-{(4″-chloro-2″,5″-dimethylbenzene)-N′-ethylsulfonamido)-phenyl]-N-[2-(N″-ethylpiperidin-4-yl)eth-1-yl]propionamide (21)

### Step a) Preparation of 2-{4-chloro-2,5-dimethylbenzene N-ethylsulfonamido}phenyl propionic acid

2-{4-chloro-2,5-dimethylbenzene N-ethylsulfonamido}phenyl propynoic acid was hydrogenated under reduced pressure, in EtOH, at 35 psi, for 6 hours. The crude mixture was filtered over celite. Upon evaporation of the solvent, the desired material was isolated as a foam.

### Step b) Preparation of 3[2′-{(4″-chloro-2″,5″-dimethylbenzene)-N′-ethyl-sulfonamido)-phenyl]-N-[2-(N″-ethylpiperidin-4-yl)eth-1-yl]propionamide

The procedure outlined in Example 1 Step e) was used substituting with 2-{4-chloro-2,5-dimethylbenzene N-ethylsulfonamido}phenyl propionic acid and 2-(N-ethyl piperidin-4-yl)ethylamine. The title material was purified by reverse phase HPLC (acetonitrile-water/0.1% TFA), and isolated as a TFA salt.

MS (ES) m/e 534 (M+H).

### Examples 22-42

Examples 22-42, which correspond to compounds 22-42 illustrated in Table II above, are synthesized using appropriate starting materials and methods described herein, including those discussed in Scheme 1 above.

### Examples 43-62

Examples 43-62, which correspond to compounds 43-62 illustrated in Table III above, are synthesized using appropriate starting materials and methods described herein, including those discussed in Scheme 1 above and Example 63 below.

### Example 62

### Preparation of 3[2′-{(4″-chloro-2″,5″-dimethylbenzene)-N′-ethylsulfonamido)-phenyl]-N-[2-(N″-ethylpiperidin-4-yl)eth-1-yl]propargylamide (63)

### Step a) Preparation of 2-iodo-N-(4-chloro-2,5-dimethylbenzene) sulfonamido phenyl

4-chloro-2,5-dimethylbenzene sulfonyl chloride was reacted with 2-iodo phenylamine in pyridine at 0° C. The reaction was warmed up to room temperature, and stirred for 12 hours. Ethyl acetate was added, and the organic layer was washed several times with 1N HCl, and brine. The organic layer was dried over MgSO₄. Upon filtration and evaporation of the solvent under reduced pressure, the title material was isolated in good yield.

### Step b) Preparation of 2-iodo-{(4-chloro-2,5-dimethylbenzene)-N-ethylsulfonamido}phenyl

2-iodo-N-(4-chloro-2,5-dimethylbenzene)sulfonamido phenyl was reacted with ethyl iodide in refluxing acetone, and K₂CO₃. The reaction mixture was refluxed for 10 hours. The solvent was evaporated under reduced pressure. EtOAc was added, and the organic layer washed with brine, dried over MgSO₄. Upon filtration and evaporation of the solvent under reduced pressure, an oil was isolated.

### Step c) Preparation of ethyl 2-{4-chloro-2,5-dimethylbenzene N-ethylsulfonamido}phenyl propynoate

The iodo analog from Step b) was reacted with ethyl propynoate in the presence of PdCl₂ (PPh₃)₂ and CuI, in DMF at 110° C., according to the procedure of Glase J. Med. Chem. 1996, 39, 3179-3187. The desired material was isolated as a foam.

### Step d) Preparation of 2-{4-chloro-2,5-dimethylbenzene N-ethyl-sulfonamido}phenyl propynoic acid

The ethyl ester from Step c) was hydrolyzed using LiOH (1.0 eq) in EtOH:H₂O. The water layer was collected and evaporated under reduced pressure. The desired material was isolated as a lithium salt.

### Step e) Preparation of 3[2′-{(4″-chloro-2″,5″-dimethylbenzene)-N′-ethyl-sulfonamido)-phenyl)-N-[2-(N″-ethylpiperidin-4-yl)eth-1-yl]propargylamide

The procedure outlined in Example 1 Step e) was used substituting 2-{4-chloro-2,5-dimethylbenzene N-ethylsulfonamido}phenyl propynoic acid and 2-(N-ethyl piperidin-4-yl)ethylamine. The title material was purified by reverse phase HPLC (acetonitrile-water/0.1% TFA).

MS (ES) m/e 530 (M+H).

The compounds shown in Table IV (compounds 64 and 65) and related compounds are made using the procedures described in the following examples.

### Example 64

### Preparation of 3-[2′-{(2″,5″-dimethyl-4″-chlorobenzene)-N′-methylsulfonamido}-phenyl]-N-[(α-(R,S)-methoxycarbonyl)benzyl]butyramide (64)

### Step a) Preparation of 4-(2-nitro)phenyl butyric acid

4-phenyl butyric acid was nitrated with HNO3, at −30 C, using the procedure described by Freedman in JACS, 71, 1949, 779.

### Step b) Preparation of 4-(2-amino)phenyl sodium butyrate

The title compound was obtained from 4-(2-nitro)phenyl butyric acid using the procedure described in Example 1, Step a).

### Step c) Preparation of 4-(2-(2,3-dichloro-benzenesulfonamido)-phenyl) butyric acid

The title compound was obtained from 4-(2-amino)phenyl sodium butyrate using the procedure described in Example 1, Step b).

### Step d) Preparation of 4-methyl-[N-methyl-2-(2,3-dichlorobenzenesulfonamido)phenyl)butyrate

The title compound was obtained from 4-(2-(2,3-dichloro-benzenesulfonamido)phenyl)butyric acid using the procedure described in Example 1, Step c).

### Step e) Preparation of 4-[N-methyl-2-(2,3-dichlorobenzenesulfonamido)phenyl)butyric acid

The title compound was obtained from 4-methyl-[N-methyl-2-(2,3-dichloro-benzenesulfonamido)phenyl)butyrate using the procedure outlined in Example 1, Step d).

### Step f) Preparation of 3-[2′-{(2″,5″-dimethyl-4″-chlorobenzene)-N′-methylsulfonamido}-phenyl]-N-[(α-methoxycarbonyl)benzyl]

The title material was obtained from 4-[N-methyl-2-(2,3-dichlorobenzene-sulfonamido)phenyl)butyric acid and a-(R,S)-methoxycarbonyl benzylamino, using the procedure described in Example 1, Step e).

MS (ES) m/e 550 (M+H).

### Example 65

### Preparation of 3-[2′-{(2″,3″-dichlorobenzene)-N′-methylsulfonamido}phenyl]-N-N-[(α-(R,S)-methoxycarbonyl)benzyl]butyramide (65)

The procedure outlined in Example 23 was used to prepare the title compound, substituting 2,3-dichlorobenzene sulfonyl chloride with 4-chloro-2,5-dimethylbenzene sulfonyl chloride.

MS(ES) m/e 544 (M+H).

### Examples 66-134

Examples 66-134, which correspond to compounds 66-134 illustrated in Table I above, are synthesized using appropriate starting materials and methods described herein, including those discussed in Scheme 1 above.

### Examples 135-138

Examples 135-138, which correspond to compounds 135-138 illustrated in Table II above, are synthesized using appropriate starting materials and methods described herein, including those discussed in Scheme 1 above.

### Examples 139

Example 139, which correspond to compound 139 illustrated in Table IV above, is synthesized using appropriate starting materials and methods described herein, including those discussed in Scheme 1 above.

### Biological Example

The potency and efficacy to inhibit the bradykinin B1 receptor was determined for the compounds of this invention in a cell-based fluorescent calcium-mobilization assay. The assay measures the ability of test compounds to inhibit B1 agonist-induced increase of intracellular free Ca⁺² in a native human B1 receptor-expressing cell line.

In this example, the following additional abbreviations have the meanings set forth below. Abbreviations heretofore defined are as defined previously. Undefined abbreviations have there art recognized meanings.
BSA=bovine serum albumin
DMSO=dimethylsulfoxide
FBS=fetal bovine serum
MEM=minimum essential medium
mM=millimolar
ng=nanogram
μg=micrograms
μM=micromolar

Specifically, calcium indicator-loaded cells are pre-incubated in the absence or presence of different concentrations of test compounds followed by stimulation with selective B1 agonist peptide while Ca-dependent fluorescence is monitored.

IMR-90 human lung fibroblast cells (CCL 186, American Type Tissue Collection) are grown in MEM supplemented with 10% FBS as recommended by ATCC. Confluent cells are harvested by trypsinization and seeded into black wall/clear bottom 96-well plates (Costar #3904) at approximately 13,000 cells/well. The following day, cells are treated with 0.35 ng/mL interleukin-1β in 10% FBS/MEM for 2 hours to up-regulate B1 receptors. Induced cells are loaded with fluorescent calcium indicator by incubation with 2.3 μM Fluo-4/AM (Molecular Probes) at 37° C. for 1.5 hrs in the presence of an anion transport inhibitor (2.5 mM probenecid in 1% FBS/MEM). Extracellular dye is removed by washing with assay buffer (2.5 mM probenecid, 0.1% BSA, 20 mM HEPES in Hank's Balanced Salt Solution without bicarbonate or phenol red, pH 7.5) and cell plates are kept in dark until used. Test compounds are assayed at 7 concentrations in triplicate wells. Serial dilutions are made in half log-steps at 100-times final concentration in DMSO and then diluted in assay buffer. Compound addition plates contain 2.5-times final concentrations of test compounds or controls in 2.5% DMSO/assay buffer. Agonist plates contain 5-times the final concentration of 2.5 mM (3×EC₅₀) B1 agonist peptide des-Arg¹⁰-kallidin (DAKD, Bachem) in assay buffer. Addition of test compounds to cell plate, incubation for 5 min at 35° C., followed by the addition of B1 agonist DAKD is carried out in the Fluorometric Imaging Plate Reader (FLIPR, Molecular Devices) while continuously monitoring Ca-dependent fluorescence. Peak height of DAKD-induced fluorescence is plotted as function of concentration of test compounds. IC₅₀ values are calculated by fitting a 4-parameter logistic function to the concentration-response data using non-linear regression (Xlfit, IDBS).

Typical potencies observed for B1 receptor agonist peptides are EC₅₀ approximately 0.8 nM and approximately 100 nM for des-Arg¹⁰-kallidin and des-Arg⁹-bradykinin, respectively, while for B1 antagonist peptide des-Arg¹⁰, Leu⁹-kallidin IC₅₀ is approximately 1 nM.

The compounds of this invention, including those of Formula I, exhibited IC₅₀ values of 0.1 to 10,000 nM in this assay.

In view of the above, all of these compounds exhibit B₁ antagonistic properties and, accordingly, are useful in treating disease conditions mediated at least in part by B1.

1. A compound of Formula I: 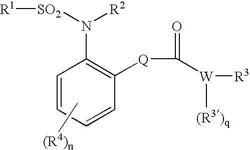
wherein
Q is selected from the group consisting of C₂-C₃ alkylene, C₂—C₃ alkenylene and C₂-C₃ alkynylene;
W is selected from the group consisting of O, S, and N, wherein:
when W is O or S, then q is zero; and when W is N, then q is one;
R¹ is selected from the group consisting of aryl, substituted aryl, heteroaryl, substituted heteroaryl, heterocyclic and substituted heterocyclic;
R² is selected from the group consisting of hydrogen, alkyl, cycloalkyl, aryl, arakyl, heteroaryl, heteroaralkyl and heterocyclic;
R³ and R^{3′} are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic and substituted heterocyclic, or R³ and R^{3′} together with the nitrogen atom to which they are attached form a heteroaryl, substituted heteroaryl, heterocyclic, or substituted heterocyclic;
each R⁴ is independently selected from the group consisting of alkyl, amino, substituted amino, cycloalkyl, alkoxy, aryl, heteroaryl, heterocyclic, acyl, halogen, nitro, cyano, hydroxy, carboxy, —C(O)OR¹⁰ wherein R¹⁰ is alkyl, substituted alkyl, aryl, or substituted aryl, and —C(O)NR¹¹R¹² wherein R¹¹ and R¹² are independently selected from the group consisting of hydrogen, alkyl, aryl, cycloalkyl, heteroaryl, and heterocyclic, or R¹¹ and R¹² together with the nitrogen atom to which they are joined form a heteroaryl, substituted heteroaryl, heterocyclic a substituted heterocyclic group;
n is an integer of from 0 to 3;
q is an integer equal to 0 or 1;
or pharmaceutically acceptable salts, prodrugs or isomers thereof.

2. The compound according to claim 1 wherein R¹ is selected from the group consisting of phenyl; naphth-1-yl; 5-dimethylaminonaphth-1-yl; 2-fluorophenyl; 2-chlorophenyl; 2-cyano-phenyl; 2-methylphenyl; 2-nitrophenyl; 2-trifluoromethylphenyl; 3-chloro-phenyl; 4-methylphenyl (tolyl); 2,5-dibromophenyl; 4-bromo-2-ethylphenyl; 4-bromo-2-trifluoromethoxyphenyl; 2,3-dichlorophenyl; 2,4-dichlorophenyl; 3,4-dichlorophenyl; 2,5-dichlorophenyl; 3,5-dichlorophenyl; 2,6-dichloro-phenyl; 2-chloro-4-cyanophenyl; 2-chloro-4-fluorophenyl; 3-chloro-2-methylphenyl; 2-chloro-6-methylphenyl; 5-chloro-2-methoxyphenyl; 2-chloro-4-trifluoromethyl-phenyl; 2,4-difluorophenyl; 5-fluoro-2-methyl-phenyl; 2,5-dimethoxyphenyl; 2-methoxy-4-methylphenyl; 2-methoxy-5-bromophenyl; 2-methoxy-5-methylphenyl; 2,5-dimethylphenyl; 2-methyl-5-nitrophenyl; 3,5-di(trifluoromethyl)phenyl; 4-bromo-2,5-difluorophenyl; 2,3,4-trichlorophenyl; 2,4,5-trichlorophenyl; 2,4,6-trichlorophenyl; 2,4-dichloro-5-methylphenyl; 4-chloro-2,5-dimethylphenyl; 2,4,6-tri(iso)propyl-phenyl; 2,4,6-trimethylphenyl; 2,3,5-trimethyl-4-chlorophenyl; 2,3,6-trimethyl-4-methoxyphenyl; 2,3,4,5,6-pentamethylphenyl; 5-chloro-1,3-dimethylpyrazol-4-yl; 2-methoxycarbonyl-thiophen-3-yl; 2,3-dimethyl-imidazol-5yl; 2-methylcarbonylamino-4-methyl-thiazol-5-yl; quinolin-8-yl; thiophen-2-yl; 1-methylimidiazol-4-yl; 3,5-dimethylisoxazol-4-yl; N-morpholino; 2,3,4-trifluoro-phenyl; 2,4-dichloro-3-methylphenyl; 2,4-dimethyl-5-chlorophenyl; 2-chloro-5-methylphenyl; 2-methyl-4-fluorophenyl; 2-phenoxyphenyl; 3-(4-methyl-phenoxy)-phenyl; 3,4-difluorophenyl; 3,4-dimethoxyphenyl; 3-chloro-4-fluorophenyl; 3-chloro-4-methylphenyl; 3-methylphenyl; and 6-chloro-5-bromopyrid-3-yl.

3. The compound according to claim 2 wherein R¹ is selected from the group consisting of 4-chloro-2,5-dimethylphenyl and 2,3-dichlorophenyl.

4. The compound according to claim 1 wherein R² is hydrogen or methyl, or ethyl.

5. The compound according to claim 1, wherein W is nitrogen and q is one.

6. The compound according to claim 5, wherein R³ is selected from the group consisting of:
amino,
2-[N-(α-aminoacetyl)piperid-4-yl]ethyl,
4-aminobenzyl,
2-[N-(1-amino-1-methylethylcarbonyl)piperid-4-yl]ethyl,
2-(4-aminophenyl)ethyl,
2-aminothiazol-5-ylmethyl,
(2-aminopyrid-4-yl)methyl,
benzyl,
2-bromoethyl,
1-(S)-carboxamide-2-(indol-3-yl)ethyl,
carboxamidemethyl,
1-carboxamide-2-(S)-methyl-butyl,
1-(S)-carbamyol-2-(phenyl)ethyl,
1-(R)-carboxamide-2-(phenyl)ethyl,
4-carboxybenzyl,
2-chloroethyl,
cyanomethyl,
2-(4-cyanophenyl)ethyl,
2-(4-cyanophenyl)-1-(R)-(pyrrolidin-N-ylcarbonyl)ethyl,
2-(4-cyanophenyl)-1-(S)-(pyrrolidin-N-ylcarbonyl)ethyl,
cyclohexyl,
cyclohexylmethyl,
2-(N-cyclopropylpiperidin-4-yl)ethyl,
2-(N-cyclopropylpiperidin-4-yl)-1-(R)-(pyrrolidin-N-ylcarbonyl)ethyl,
1-(R)-1,3-di(benzyloxycarbonyl)propyl,
1-(S)-1,3-dicarboxamidepropyl,
(2-dimethylamino)ethyl,
2-[4-(N,N-dimethylamino]phenethyl,
3-(dimethylamino)propyl,
1-(S)-ethoxycarbonylethyl,
2-ethoxyphenyl,
ethyl,
1-(R)-(1-N-ethylaminocarbonyl)-4-amino-n-butyl,
1-(S)-(1-N-ethylaminocarbonyl)-4-amino-n-butyl,
1-(R)-(1-N-ethylaminocarbonyl)-5-(t-butoxycarbonylamino)pent-5-yl,
1-(S)-(1-N-ethylaminocarbonyl)-5-(t-butoxycarbonylamino)pent-5-yl,
1-(R)-(1-N-ethylaminocarbonyl)-4-(N′-t-butoxycarbonylamino)-n-but-5-yl,
1-(S)-(1-N-ethylaminocarbonyl)-4-(N′-t-butoxycarbonylamino)-n-but-5-yl,
1-(R)-(1-N-ethylaminocarbonyl)-5-(N′-t-butoxycarbonylamino)-n-pent-5-yl,
1-(S)-(1-N-ethylaminocarbonyl)-5-(N′-t-butoxycarbonylamino)-n-pent-5-yl,
4-fluorophenethyl,
hydrogen,
2-hydroxyethyl,
2-(4-hydroxyphenyl)-1-(S)-(methoxycarbonyl)ethyl,
2-(4-hydroxyphenyl)-1-(S)-(isopropoxycarbonyl)ethyl,
2-(4-hydroxyphenyl)-1-(R)-(methoxycarbonyl)ethyl,
2-(N-hydroxypyrid-4-yl)ethyl,
2-(imidazol-4-yl)ethyl,
2-[4-(imidazolin-2-yl)phenyl]-1-(R)-(pyrrolidin-1-ylcarbonyl)ethyl,
2-[4-(imidazolin-2-yl)phenyl]ethyl,
2-(indol-3-yl)ethyl,
2-(indol-3-yl)-1-(S)-(methoxycarbonyl)ethyl,
2-(indol-3-yl)-1-(R)-(methoxycarbonyl)ethyl,
iso-propyl,
1-(R)-(isopropoxycarbonyl)-2-(phenyl)ethyl,
4-(methoxycarbonyl)benzyl,
1-(R)-(methoxycarbonyl)ethyl,
methoxycarbonylmethyl,
methoxycarbonylphenylmethyl,
2-methoxyethyl,
1-(R)-(methoxcarbonyl)-2-(N-methylpiperidin-4-yl)ethyl,
1-(R)-(methoxycarbonyl)-2-(N-methyl-1,2,3,6-tetrahydropyrid-4-yl)ethyl,
2-methoxyphenyl,
1-(R)-(methoxycarbonyl)-2-pyrid-4-yl)ethyl,
methyl,
2-[4-(methylcarbonylamino]phenethyl,
2-(4-methylpiperazin-1-yl)ethyl,
2-(N-methylpiperidin-4-yl)ethyl,
(N-methylpiperidin-2-yl)methyl,
2-(N-methylpiperidin-2-yl)ethyl,
2-(N-methylpiperidin-3-yl)ethyl,
2-(N-methylpiperidin-4-yl)ethyl,
2-(N-methylpiperidin-4-yl)-1-(R)-(pyrrolidin-N-ylcarbonyl)ethyl,
2-[(N-methyl)pyrrolidin-2-yl]ethyl,
2-(N-methyl-1,2,5,6-tetrahydropyrid-4-yl)ethyl,
2-(N-methyl-1,2,5,6-tetrahydropyrid-4-yl)-1-(R)-(pyrrolidin-N-ylcarbonyl)ethyl,
3-(2-methylthiazol-5-yl)-pyrazol-5-yl,
2-(N-morpholino)ethyl,
n-hexyl,
4-nitrobenzyl,
phenethyl,
1-(R)-phenylethyl,
1-(S)-phenylethyl,
phenyl,
4-phenylbutyl,
1-(R)-2-phenylcarboxyethyl,
1-(R)-2-phenyl-1-(methoxycarbonyl)ethyl,
1-(S)-2-phenyl-1-(methoxycarbonyl)ethyl,
3-phenyl-n-propyl,
2-(phenyl)-1-(S)-(pyrrolidin-N-ylcarbonyl)ethyl,
2-(piperidin-N-yl)ethyl,
2-(piperidin-2-yl)ethyl,
2-(piperidin-3-yl)ethyl,
2-(piperidin-4-yl)ethyl,
(piperid-1-yl)carbonylmethyl,
pyrazin-2-ylmethyl,
2-(pyrid-2-yl)ethyl,
2-(pyrid-3-yl)ethyl,
2-(pyrid-4-yl-)ethyl,
(pyrid-2-yl)methyl,
(pyrid-3-yl)methyl,
(pyrid-4-yl)methyl,
2-[N-(pyrid-4-yl)]piperidin-4-yl]ethyl,
2-[N-(pyrid-3-yl)piperidin-4-yl)]ethyl,
2-[N-(pyrid-2-yl)piperidin-4-yl]ethyl,
2-[N-(4-methylpyrid-2-yl)]piperidin-4-yl]ethyl,
2-[N-(3-methylpyrid-2-yl)]piperidin-4-yl]ethyl,
2-(pyrid-4-yl)-1-(R)-(pyrrolidin-N-ylcarbonyl)ethyl,
1-(R)-(pyrrolidin-N-ylcarbonyl)-5-amino-n-pentyl,
1-(S)-(pyrrolidin-N-ylcarbonyl)-5-amino-n-pentyl,
1-(R)-(pyrrolidin-N-ylcarbonyl)-2-(4-biphenyl)ethyl,
1-(S)-(pyrrolidin-N-ylcarbonyl)-2-(4-biphenyl)ethyl,
1-(R)-(pyrrolidin-N-ylcarbonyl-2-(4-iodophenyl)ethyl,
1-(S)-(pyrrolidin-N-ylcarbonyl-2-(4-iodophenyl)ethyl,
1-(R)-(pyrrolidin-N-carbonyl)-4-(t-butoxycarbonylamino)-n-butyl,
1-(S)-(pyrrolidin-N-carbonyl)-4-(t-butoxycarbonylamino)-n-butyl,
1-(S)-(pyrrolidin-N-ylcarbonyl)-2-[4-(2-imidazolin-2-yl)phenyl]ethyl,
2-(R)-(pyrrolidin-N-ylcarbonyl-3-phenylprop-2-yl,
1-(R)-(pyrrolidin-N-ylcarbonyl)-2-[4-(N-methylpiperidin-2-yl)phenyl)]ethyl,
1-(S)-(pyrolidin-N-ylcarbonyl)-2-[4-(N-methylpiperidin-2-1)phenyl)]ethyl,
1-(R)-(pyrrolidin-N-ylcarbonyl)-2-[N-methyl-1,2,5,6-tetrahydro-pyridin-4-yl)-phen-4-yl)]ethyl,
1-(S)-(pyrrolidin-N-ylcarbonyl)-2-[N-methyl-1,2,5,6-tetrahydro-pyridin-4-yl)-phen-4-yl)]ethyl,
1-(R)-(pyrrolidin-N-ylcarbonyl)-2-[4-(piperidin-2-yl)cyclohexyl)]ethyl,
1-(S)-(pyrrolidin-N-ylcarbonyl)-2-[4-(piperidin-2-yl)cyclohexyl)]ethyl,
1-(R)-(pyrrolidin-N-ylcarbonyl)-2-[N-(phenyl)piperidin-4-yl)]ethyl,
1-(S)-(pyrrolidin-N-ylcarbonyl)-2-[N-(phenyl)piperidin-4-yl)]ethyl,
1-(R)-(pyrrolidin-N-ylcarbonyl)-2-[N-(pyridin-4-yl)piperidin-4-yl)]ethyl,
1-(S)-(pyrrolidin-N-ylcarbonyl)-2-[N-(pyridin-4-yl)piperidin-4-yl)]ethyl,
1-(R)-(pyrrolidin-N-ylcarbonyl)-2-[4-(pyridin-4-yl)phenyl)]ethyl,
1-(S)-(pyrrolidin-N-ylcarbonyl)-2-[4-(pyridin-4-yl)phenyl)]ethyl,
1-(R)-(pyrrolidin-N-ylcarbonyl)-2-[4-(pyrid-2-yl)phenyl]ethyl,
1-(S)-(pyrrolidin-N-ylcarbonyl)-2-[4-(pyrid-2-yl)phenyl]ethyl,
1-(R)-(pyrrolidin-N-ylcarbonyl)-2-[4-(pyrimidin-2-yl)phenyl]ethyl,
1-(S)-(pyrrolidin-N-ylcarbonyl)-2-[4-(pyrimidin-2-yl)phenyl]ethyl,
1-(R)-(pyrrolidin-N-ylcarbonyl)-2-[4-(N-t-butoxycarbonylpyrrol-2-yl)phenyl]ethyl,
1-(S)-(pyrrolidin-N-ylcarbonyl)-2-[4-(N-t-butoxycarbonylpyrrol-2-yl)phenyl]ethyl,
1-(S)-(t-butoxycarbonyl)-2-(4-hydroxyphenyl)ethyl,
3-t-butoxycarbonyl-1-methoxycarbonylpropyl,
2-[N-(t-butoxycarbonylmethyl)piperid-4-yl]ethyl,
2-[1-(t-butoxycarbonylmethyl)piperid-4-yl)]ethyl,
1-(S)-(t-butoxycarbonyl)-3-methylpropyl,
1-(R)-(t-butoxycarbonyl)-3-methylpropyl,
1-(R)-(t-butoxycarbonyl)-2-(phenyl)ethyl,
2-(N-t-butoxycarbonylmethyl)pyridin-4-yl-ethyl,
1-R-(N-pyrrolidinylcarbon-yl)-2-(4-pyridyl)ethyl,
1-S-(N-pyrrolidinylcarbon-yl)-2-(4-pyridyl)ethyl,
1-R-1-(N-piperidinylcarbonyl)ethyl,
1-S-1-(N-piperidinylcarbonyl)ethyl,
1-R-1-methyl-2-(N-piperidinyl)ethyl,
1-S-1-methyl-2-(N-piperidinyl)ethyl,
1-R-1-methyl-2-(4-methylpiperazin-1-yl)ethyl,
1-S-1-methyl-2-(4-methylpiperazin-1-yl)ethyl,
α-(R,S,)-methoxycarbonylbenzyl,
1(R)-1-[4-methylpiperazinylcarbonyl]ethyl,
1(S)-1-[4-methylpiperazinylcarbonyl]ethyl,
2-(N-(5-methyl-pyrimidin-4-yl)-piperidin-4-yl)ethyl,
2-(N-(pyrimidin-4-yl)-piperidin-4-yl)ethyl,
2-(N,N-dimethylpiperidin-4-yl)ethyl,
2-[4-(piperidinylmethyl)phenyl]ethyl,
2-oxo-5-phenyl-2,3-dihydro-1H-benzo[e][1,4]diazepin-3-yl, and
4-[1-(pyrid-4-yl)-piperazin-4-yl]phenyl.

7. The compound according to claim 5, wherein R^{3′} is selected from the group consisting of hydrogen, methyl, ethyl, iso-propyl, 2-methoxyethyl, and pyrid-3-ylmethyl.

8. The compound according to claim 5, wherein R^{3′} is hydrogen.

9. The compound according to claim 5 wherein q is zero and W is oxygen or sulfur.

10. The compound according to claim 5, wherein R³ and R^{3′} are joined, together with the nitrogen atom to which they are bound, to form an optionally substituted heterocyclic group.

11. The compound according to claim 10, wherein said optionally substituted heterocyclic group is selected from the group consisting of 4-(2-aminoethyl)-piperidin-1-yl; 4-[2-(N-t-butoxycarbonylamino)ethyl]piperidin-1-yl; 1-(pyridin-2-yl)piperazin-4-yl; N-morpholino; 2-methylpiperid-N-yl; 2-(S)-carboxamide-pyrrolidin-N-yl; 2-(R)-hydroxy-5-(S)-methoxycarbonyl-pyrrolidin-N-yl; 2-(R)-methoxycarbonyl-pyrrolidin-N-yl; 2-(S)-methoxy-methylpyrrolidin-1-yl; 3-(R)-(t-butoxycarbox-amido)pyrrolidin-N-yl; 3-carboxamidepiperid-N-yl; 3-hydroxypyrrolidin-N-yl; 4-acetylpiperazin-1-yl; 4-hydroxypiperid-N-yl; and 4-methylpiperazin-1-yl.

12. The compound according to claim 1, wherein n is zero and each R⁴ is hydrogen.

13. The compound according to claim 1, wherein n is an integer from 1 to 3.

14. The compound according to claim 13, wherein is 2 and each R⁴ is independently selected from the group consisting of fluoro, chloro and methyl.

15. The compound according to claim 1 which compound is represented by formula IIa: 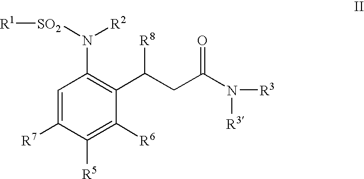
and pharmaceutically acceptable salts thereof.

16. A compound according to claim 15 which is selected from the group consisting of:
3-[2′-{(4″-chloro-2″,5″-dimethylbenzene)-N′-methylsulfonamido}-phenyl]-N-[1-(R)-1-(N″-pyrrolidinylcarbonyl)-2-(4-pyridyl)eth-1-yl]propionamide (1);
3-[2′-{(4″-chloro-2″,5″-dimethylbenzene)-N′-methylsulfonamido}-phenyl]-N-[2-(N″-methylpiperidin-4-yl)eth-1-yl]propionamide (2);
3-[2′-{(4″-chloro-2″,5″-dimethylbenzene)-N′-methylsulfonamido}-phenyl]-N-[1-(R)-1-(N″-piperidinylcarbonyl]eth-1-yl]propionamide (3);
3-[2′-{(4″-chloro-2″,5″-dimethylbenzene)-N′-methylsulfonamido}-phenyl]-N-[1-(S)-1-(N″-piperidinylcarbonyl]eth-1-yl]propionamide (4);
3-[2′-{(4″-chloro-2″,5″-dimethylbenzene)-N′-methylsulfonamido}-phenyl]-N-[2-{N″-(4-methylpyrid-2-yl)}piperidin-4-yl]eth-1-yl]propion-amide (5);
3-[2′-{(4″-chloro-2″,5″-dimethylbenzene)-N′-methylsulfonamido}-phenyl]-N-[1-(R)-1-(N″-pyrrolidinylcarbonyl)-2-(4-pyridylphen-4-yl)eth-1-yl]propionamide (6);
3-[2′-{(4″-chloro-2″,5″-dimethylbenzene)-N′-methylsulfonamido}-phenyl]-N-[(2-N″-piperidinyl)eth-1-yl]propionamide (7);
3-[2′-{(4″-chloro-2″,5″-dimethylbenzene)-N′-methylsulfonamido}-phenyl]-N-[(2-pyrid-4-yl)eth-1-yl]propionamide (8);
3-[2′-{(4″-chloro-2″,5″-dimethylbenzene)-N′-methylsulfonamido}-phenyl]-N-[2-{N″-{(2-pyridyl)piperidin-4-yl}eth-1-yl]propionamide (9);
3-[2′-{(4″-chloro-2″,5″-dimethylbenzene)-N′-methylsulfonamido}-phenyl]-N-[2-(N″-ethylpiperidin-4-yl)eth-1-yl]propionamide (10);
3-[2′-{(4″-chloro-2″,5″-dimethylbenzene)-N′-methylsulfonamido}-phenyl]-N-[1-(S)-1-methyl-2-(N″-piperidinyl)eth-1-yl]propionamide (11);
3-[2′-{(4″-chloro-2″,5″-dimethylbenzene)-N′-methylsulfonamido}-phenyl]-N-[2-{N″-(pyrid-4-yl}piperidin-4-yl)eth-1-yl]propionamide (12);
3-[2′-{(4″-chloro-2″,5″-dimethylbenzene)-N′-methylsulfonamido}-phenyl]-N-[1-(R)-1-methyl-2-(N″-piperidinyl)eth-1-yl]propionamide (13);
3-[2′-{(4″-chloro-2″,5″-dimethylbenzene)-N′-methylsulfonamido}-phenyl]-N-[2-{N″-(2-methylpyrid-4-yl}piperidin-4-yl)eth-1-yl]propion-amide (14);
3-[2′-{(4″-chloro-2″,5″-dimethylbenzene)-N′-methylsulfonamido}-phenyl]-N-[1-(S)-1-methyl-2-(4-methylpiperazin-1-yl)eth-1-yl]propion-amide (15);
3-[2′-{(4″-chloro-2″,5″-dimethylbenzene)-N″-methylsulfonamido}-phenyl]-N-[1-(R)-1-methyl-2-(4-methylpiperazin-1-yl)eth-1-yl]propionamide (16);
3-[2′-{(4″-chloro-2″,5″-dimethylbenzene)-N′-methylsulfonamido}-phenyl]-N-methyl-N-2-(N″-methylpiperidin-4-yl)eth-1-yl]propionamide (17);
3-[2′-{(2″,3″-dichlorobenzene)-N′-methylsulfonamido}phenyl]-N-methyl-N-2-[(N″-methylpiperidin-4-yl)eth-1-yl]propionamide (18);
3-[2′-{(2″,5″-dimethyl-4″-chlorobenzene)-N′-methylsulfonamido}-phenyl]-N-[(α-(R,S)-methoxycarbonyl)benzyl]propionamide (19);
3-[2′-{(2″,3″-dichlorobenzene)-N′-methylsulfonamido}phenyl]-N-N-[(α-(R,S)-methoxycarbonyl)benzyl]propionamide (20); and
3-[2′-{(4″-chloro-2″,5″-dimethylbenzene)-N′-ethylsulfonamido}-phenyl]-N-[2-(N″-ethylpiperidin-4-yl)eth-1-yl]propionamide (21);
3-[2′-{(4″-chloro-2″,5″-dimethylbenzene)sulfonamido}-phenyl]-N-[2-(N″-methylpiperidin-4-yl)ethyl]propionamide (66);
3-[2′-{(2″,3″-dichlorobenzene)sulfonamido}-phenyl]-N-[2-(N″-methylpiperidin-4-yl)ethyl]propionamide (67);
3-[2′-{(4″-chloro-2″,5″-dimethylbenzene)-N′-methylsulfonamido}-phenyl]-N-[1(R or S)-1-(4-methyl piperazinylcarbonyl)ethyl]propion-amide (68);
3-[2′-{(4″-chloro-2″,5″-dimethylbenzene)N′-methylsulfonamido}-phenyl]-N-[1(S or R)-1-(4-methyl piperazinylcarbonyl)ethyl]propion-amide (69);
3-[2′-{(4″-chloro-2″,5″-dimethylbenzene)-N′-methylsulfonamido}-phenyl]-N-[2-{N″-(3-methylpyrid-2-yl)}piperidin-4-yl]ethyl]propion-amide (70);
3-[2′-{(4″-chloro-2″,5″-dimethylbenzene)-N′-methylsulfonamido}-phenyl]-N-[2-(N″,N″-dimethylpiperidin-4-yl)ethyl]propionamide (71);
3-[2′-{(4″-chloro-2″,5″-dimethylbenzene)sulfonamido}-4,5-dichlorophenyl]-N-methyl-N-[2-(N″-methylpiperidin-4-yl)ethyl]propionamide (72);
3-[2′-{(3″-methyl-2″,4″-dichlorobenzene)sulfonamido}-4,5-dichlorophenyl]-N-methyl-N-[2-(N″-methylpiperidin-4-yl)ethyl]propionamide (73);
3-[2′-{(4″-chloro-2″,5″-dimethylbenzene)-N′-ethylsulfonamido}phenyl]-N-methyl-N-[2-[N″-(pyrid-2-yl)piperidin-4-yl]ethyl]propionamide (74);
3-[2′-{(3″-(4″″-methylphenoxy)benzene)-N′-methylsulfonamido}phenyl]-N-[2-(N″-methylpiperidin-4-yl)ethyl]propionamide (75);
3-[2′-{(4″-chloro-2″,5″-dimethylbenzene)-N′-ethylsulfonamido}phenyl]-N-ethyl-N-[2-[N″-(pyrid-2-yl)piperidin-4-yl]ethyl]propionamide (76);
3-[2′-{(4″-chloro-2″,5″-dimethylbenzene)-N′-ethylsulfonamido}phenyl]-N-[2-oxo-5-phenyl-2,3-dihydro-1H-benzo[e][1,4]diazepin-3-yl]propionamide (77);
3-[2′-{(4″-chloro-2″,5″-dimethylbenzene)-N′-methylsulfonamido}-5,6-dichlorophenyl]-N-methyl-N-[2-(N″-methylpiperidin-4-yl)ethyl]propionamide (78);
3-[2′-{(4″-chloro-2″,5″-dimethylbenzene)-N′-methylsulfonamido}-4,5-dichlorophenyl]-N-[2-(N″-methylpiperidin-4-yl)ethyl]propionamide (79);
3-[2′-{(4″-chloro-2″,5″-dimethylbenzene)-N′-methylsulfonamido}-5,6-difluorophenyl]-N-methyl-N-[2-(N″-methylpiperidin-4-yl)ethyl]propionamide (80);
3-[2′-{(4″-chloro-2″,5″-dimethylbenzene)-N′-ethylsulfonamido}phenyl]-N-isopropyl-N-[2-(N″-methylpiperidin-4-yl)ethyl]propionamide (81);
3-[2′-{(4″-chloro-2″,5″-dimethylbenzene)-N′-methylsulfonamido}-5,6-difluorophenyl]-N-methyl-N-[2-(N″-methylpiperidin-4-yl)ethyl]propionamide (82);
3-methyl-3-[2′-{(4″-chloro-2″,5″-dimethylbenzene)-N′-methylsulfonamido}phenyl]-N-[2-{N″-(pyrid-4-yl}piperidin-4-yl)ethyl]propionamide(Racemic mixture) (83);
3-methyl-3-[2′-{(4″-chloro-2″,5″-dimethylbenzene)-N′-methylsulfonamido}phenyl]-N-[2-{N″-(pyrid-4-yl}piperidin-4-yl)ethyl]propionamide (Isomer A, of racemic mixture) (84);
3-methyl-3-[2′-{(4″-chloro-2″,5″-dimethylbenzene)-N′-methylsulfonamido}phenyl]-N-[2-{N″-(pyrid-4-yl}piperidin-4-yl)ethyl]propionamide (Isomer B, of racemic mixture) (85);
3-[2′-{(4″-methylbenzene)sulfonamido}phenyl]-N-[2-(N″,N″-dimethylpiperidin-4-yl)ethyl]propionamide (86);
3-[2′-{(3″-chloro-4″-methylbenzene)sulfonamido}-phenyl]-N-[2-(N″,N″-dimethylpiperidin-4-yl)ethyl]propionamide (87);
3-[2′-{(2″-methyl-3″-chlorobenzene)sulfonamido}phenyl]-N-[2-(N″,N″-dimethylpiperidin-4-yl)ethyl]propionamide (88);
3-[2′-{(3″,4″-dichlorobenzene)-N′-methylsulfonamido}-phenyl]-N-[2-(N″,N″-dimethylpiperidin-4-yl)ethyl]propionamide (89);
3-[2′-{(2″-fluorobenzene)sulfonamido}phenyl]-N-[2-(N″,N″-dimethylpiperidin-4-yl)ethyl]propionamide (90);
3-methyl-3-[2′-{(4″-chloro-2″,5″-dimethylbenzene)-N′-methylsulfonamido}phenyl]-N-[2-(N″-methylpiperidin-4-yl)ethyl]propionamide (91);
3-[2′-{(4″-chloro-2″,5″-dimethylbenzene)sulfonamido}-phenyl]-N-[2-(N″,N″-dimethylpiperidin-4-yl)ethyl]propionamide (92);
3-methyl-3-[2′-{(4″-chloro-2″,5″-dimethylbenzene)-N′-methylsulfonamido}-phenyl]-N-[2-{N″-(5-methylpyrid-2-yl)}piperidin-4-yl]ethyl]propionamide (93);
3-[2′-{(2″,4″-dichlorobenzene)-N′-methylsulfonamido}-phenyl]-N-[2-(N″,N″-dimethylpiperidin-4-yl)ethyl]propionamide (94);
3-[2′-{(4″-chloro-2″,5″-dimethylbenzene)-N′-ethylsulfonamido}-phenyl]-N-[2-(N″-methylpiperidin-4-yl)ethyl]propionamide (95);
3-[2′-{(4″-chloro-2″,5″-dimethylbenzene)-N′-ethylsulfonamido}-phenyl]-N-[2-(N″-methylpiperidin-2-yl)ethyl]propionamide (96);
3-[2′-{(4″-chloro-2″,5″-dimethylbenzene)-N′-ethylsulfonamido}-phenyl]-N-[2-(N″-methylpiperidin-3-yl)ethyl]propionamide (97);
3-[2′-{benzene-N′-methylsulfonamido}-phenyl]-N-[2-(N″,N″-dimethylpiperidin-4-yl)ethyl]propionamide (98);
3-[2′-{(2″,3″-dichlorobenzene)-N′-methylsulfonamido}-phenyl]-N-[2-(N″,N″-dimethylpiperidin-4-yl)ethyl]propionamide (99);
3-[2′-{(2″-chlorobenzene)-N′-methylsulfonamido}-phenyl]-N-[2-(N″,N″-dimethylpiperidin-4-yl)ethyl]propionamide (100);
3-[2′-{(2″-methylbenzene)-N′-methylsulfonamido}-phenyl]-N-[2-(N″,N″-dimethylpiperidin-4-yl)ethyl]propionamide (101);
3-[2′-{(3″-chlorobenzene)-N′-methylsulfonamido}-phenyl]-N-[2-(N″,N″-dimethylpiperidin-4-yl)ethyl]propionamide (102);
3-[2′-{(3″-methylbenzene)-N′-methylsulfonamido}-phenyl]-N-[2-(N″,N″-dimethylpiperidin-4-yl)ethyl]propionamide (103);
3-[2′-{(2″,4″,5″-trichlorobenzene)-N′-methylsulfonamido}-phenyl]-N-[2-(N″,N″-dimethylpiperidin-4-yl)ethyl]propionamide (104);
3-[2′-{(2″,4″-dichloro-5″-methylbenzene)-N′-methylsulfonamido}-phenyl]-N-[2-(N″,N″-dimethylpiperidin-4-yl)ethyl]propionamide (105);
3-[2′-{(2″,5″-dimethylbenzene)-N′-methylsulfonamido}-phenyl]-N-[2-(N″,N″-dimethylpiperidin-4-yl)ethyl]propionamide (106);
3-[2′-{(2″,5″-dichlorobenzene)-N′-methylsulfonamido}-phenyl]-N-[2-(N″,N″-dimethylpiperidin-4-yl)ethyl]propionamide (107);
3-[2′-{(2″,6″-dichlorobenzene)-N′-methylsulfonamido}-phenyl]-N-[2-(N″,N″-dimethylpiperidin-4-yl)ethyl]propionamide (108);
3-[2′-{(2″-methoxy-5″-chlorobenzene)-N′-methylsulfonamido}-phenyl]-N-[2-(N″,N″-dimethylpiperidin-4-yl)ethyl]propionamide (109);
3-[2′-{(2″-methyl-5″-fluorobenzene)-N′-methylsulfonamido}-phenyl]-N-[2-(N″,N″-dimethylpiperidin-4-yl)ethyl]propionamide (110);
3-[2′-{(2″-chloro-6″-methylbenzene)-N′-methylsulfonamido}-phenyl]-N-[2-(N″,N″-dimethylpiperidin-4-yl)ethyl]propionamide (111);
3-[2′-{(3″-fluoro-4″-methylbenzene)-N′-methylsulfonamido}-phenyl]-N-[2-(N″,N″-dimethylpiperidin-4-yl)ethyl]propionamide (112);
3-[2′-{(naphthal-2-yl)-N′-methylsulfonamido}-phenyl]-N-[2-(N″,N″-dimethylpiperidin-4-yl)ethyl]propionamide (113);
3-[2′-{(3″,4″-difluorobenzene)-N′-methylsulfonamido}-phenyl]-N-[2-(N″,N″-dimethylpiperidin-4-yl)ethyl]propionamide (114);
3-[2′-{(3″-chloro-4″-fluorobenzene)-N′-methylsulfonamido}-phenyl]-N-[2-(N″,N″-dimethylpiperidin-4-yl)ethyl]propionamide (115);
3-[2′-{(3″,4″-dimethoxybenzene)-N′-methylsulfonamido}-phenyl]-N-[2-(N″,N″-dimethylpiperidin-4-yl)ethyl]propionamide (116);
3-[2′-{(2″-chloro-4″-cyanobenzene)-N′-methylsulfonamido}-phenyl]-N-[2-(N″,N″-dimethylpiperidin-4-yl)ethyl]propionamide (117);
3-[2′-{(2″,4″-dichloro-5″-methylbenzene)-N′-methylsulfonamido}-phenyl]-N-[2-(N″,N″-dimethylpiperidin-4-yl)ethyl]propionamide (118);
3-[2′-{(4″-chloro-2″,5″-dimethylbenzene)-N′-methylsulfonamido}phenyl]-N-methyl-N-[2-{N″-(4-methylpyrid-2-yl)}piperidin-4-yl]ethyl]propionamide (119);
3-[2′-{(4-chloro-2″,5″-dimethylbenzene)-N′-methylsulfonamido}-phenyl]-N-[2-(N″,N″-dimethylpiperidin-4-yl)ethyl]propionamide (120);
3-[2′-{(naphthalyl)-N′-methylsulfonamido}-phenyl]-N-[2-(N″,N″-dimethylpiperidin-4-yl)ethyl]propionamide (121);
3-[2′-{(4-chlorobenzo[c][1,2,5]oxadiazol-7-yl)-N′-methylsulfonamido}-phenyl]-N-[2-(N″,N″-dimethylpiperidin-4-yl)ethyl]propionamide (122);
3-[2′-{(2″-phenoxybenzene)-N′-methylsulfonamido}-phenyl]-N-[2-(N″,N″-dimethylpiperidin-4-yl)ethyl]propionamide (123);
3-[2′-{(2″,3″,4″-trifluorobenzene)-N′-methylsulfonamido}-phenyl]-N-[2-(N″,N″-dimethylpiperidin-4-yl)ethyl]propionamide (124);
3-[2′-{(2″-chloro-4″-trifluoromethylbenzene)-N′-methylsulfonamido}-phenyl]-N-[2-(N″,N″-dimethylpiperidin-4-yl)ethyl]propionamide (125);
3-[2′-{(2″-methyl-4″-fluorobenzene)-N′-methylsulfonamido}-phenyl]-N-[2-(N″,N″-dimethylpiperidin-4-yl)ethyl]propionamide (126);
3-[2′-{(3″,5″-dichloropryid-2-yl)-N′-methylsulfonamido}-phenyl]-N-[2-(N″,N″-dimethylpiperidin-4-yl)ethyl]propionamide (127);
3-[2′-{(2″,3″,4″-trichlorobenzene)-N′-methylsulfonamido}-phenyl]-N-[2-(N″,N″-dimethylpiperidin-4-yl)ethyl]propionamide (128);
3-[2′-{(3″,5″-dichlorobenzene)-N′-methylsulfonamido}-phenyl]-N-[2-(N″,N″-dimethylpiperidin-4-yl)ethyl]propionamide (129);
3-[2′-{(4′-chloro-2″,5″-dimethylbenzene)-N′-ethylsulfonamido}phenyl]-N-methyl-N-[2-[N″-(pyrimidin-4-yl)piperidin-4-yl]ethyl]propionamide (130);
3-[2′-{(4″-chloro-2″,5″-dimethylbenzene)-N′-ethylsulfonamido}phenyl]-N-methyl-N-[2-[N″-(5-methylpyrimidin-4-yl)piperidin-4-yl]ethyl]propionamide (131);
3-[2′-{(4″-chloro-2″,5″-dimethylbenzene)-N′-methylsulfonamido}phenyl]-N-methyl-N-[2-[4-(piperidinylmethyl)phenyl]ethyl]propionamide (132);
3-[2′-{(4″-chloro-2″,5″-dimethylbenzene)-N′-methylsulfonamido}phenyl]-N-methyl-N-{2-[3H-pyrrolo[3,2-b]pyrid-2-yl]ethyl}propionamide (133); and
3-[2′-{(2″,6″-dichlorobenzene)-N′-ethylsulfonamido}-phenyl]-N-[2-(N″-methylpiperidin-4-yl)ethyl]propionamide (134);
and pharmaceutically acceptable salts thereof.

17. The compound according to claim 1 which compound is represented by formula IIIa: 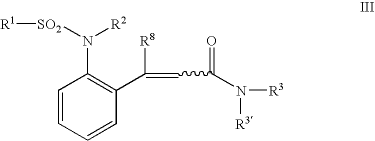
and pharmaceutically acceptable salts thereof.

18. A compound according to claim 15 which is selected from the group consisting of:
3-[2′-{(4″-chloro-2″,5″-dimethylbenzene)-N′-methylsulfonamido}-phenyl]-N-[1-(R)-1-(N″-pyrrolidinylcarbonyl)-2-(4-pyridyl)eth-1-yl]acrylamide (22);
3-[2′-{(4″-chloro-2″,5″-dimethylbenzene)-N′-methylsulfonamido}-phenyl]-N-[2-(N″-methylpiperidin-4-yl)eth-1-yl]acrylamide (23);
3-[2′-{(4″-chloro-2″,5″-dimethylbenzene)-N′-methylsulfonamido}-phenyl]-N-[1-(R)-1-(N″-piperidinylcarbonyl]eth-1-yl]acrylamide (24);
3-[2′-{(4″-chloro-2″,5″-dimethylbenzene)-N′-methylsulfonamido}-phenyl]-N-[1-(S)-1-(N″-piperidinylcarbonyl]eth-1-yl]acrylamide (25);
3-[2′-{(4″-chloro-2″,5″-dimethylbenzene)-N′-methylsulfonamido}-phenyl]-N-[2-{N″-(4-methylpyrid-2-yl)}piperidin-4-yl]eth-1-yl]acryl-mide (26);
3-[2′-{(4″-chloro-2″,5″-dimethylbenzene)-N′-methylsulfonamido}-phenyl]-N-[1-(R)-(N″-pyrrolidinylcarbonyl)-2-(4-pyridylphen-4-yl)eth-1-yl]acrylamide (27);
3-[2′-{(4″-chloro-2″,5″-dimethylbenzene)-N′-methylsulfonamido}-phenyl]-N-[(2-N″-piperidinyl)eth-1-yl]acrylamide (28);
3-[2′-{(4″-chloro-2″,5″-dimethylbenzene)-N′-methylsulfonamido}-phenyl]-N-[(2-pyrid-4-yl)eth-1-yl]acrylamide (29);
3-[2′-{(4″-chloro-2″,5″-dimethylbenzene)-N′-methylsulfonamido}-phenyl]-N-[2-{N″-{(2-pyridyl)piperidin-4-yl}eth-1-yl]acrylamide (30);
3-[2′-{(4″-chloro-2″,5″-dimethylbenzene)-N′-methylsulfonamido}-phenyl]-N-[2-(N″-ethylpiperidin-4-yl)eth-1-yl]acrylamide (31);
3-[2′-{(4″-chloro-2″,5″-dimethylbenzene)-N′-methylsulfonamido}-phenyl]-N-[1-(S)-1-methyl-2-(N″-piperidinyl)eth-1-yl]acrylamide (32);
3-[2′-{(4″-chloro-2″,5″-dimethylbenzene)-N′-methylsulfonamido}-phenyl]-N-[2-{N″-(pyrid-4-yl}piperidin-4-yl)eth-1-yl]acrylamide (33);
3-[2′-{(4″-chloro-2″,5″-dimethylbenzene)-N′-methylsulfonamido}-phenyl]-N-[1-(R)-1-methyl-2-(N″-piperidinyl)eth-1-yl]acrylamide (34);
3-[2′-{(4″-chloro-2″,5″-dimethylbenzene)-N′-methylsulfonamido}-phenyl]-N-[2-{N″-(2-methylpyrid-4-yl}piperidin-4-yl)eth-1-yl]acrylamide (35);
3-[2′-{(4″-chloro-2″,5″-dimethylbenzene)-N′-methylsulfonamido}-phenyl]-N-[1-(S)-1-methyl-2-(4-methylpiperazin-1-yl)eth-1-yl]acryl-amide (36);
3-[2′-{(4″-chloro-2″,5″-dimethylbenzene)-N′-methylsulfonamido}-phenyl]-N-[1-(R)-1-methyl-2-(4-methylpiperazin-1-yl)eth-1-yl]acryl-amide (37);
3-[2′-{(4″-chloro-2″,5″-dimethylbenzene)-N′-methylsulfonamido}-phenyl]-N-methyl-N-2-(N″-methylpiperidin-4-yl)eth-1-yl]acrylamide (38);
3-[2′-{(2″,3″-dichlorobenzene)-N′-methylsulfonamido}phenyl]-N-methyl-N-2-[(N″-methylpiperidin-4-yl)eth-1-yl]acrylamide (39);
3-[2′-{(2″,5″-dimethyl-4″-chlorobenzene)-N′-methylsulfonamido}-phenyl]-N-[(α-(R,S)-methoxycarbonyl)benzyl]acrylamide (40);
3-[2′-{(2″,3″-dichlorobenzene)-N′-methylsulfonamido}phenyl]-N-N-[(α-(R,S)-methoxycarbonyl)benzyl]acrylamide (41); and
3-[2′-{(4″-chloro-2″,5″-dimethylbenzene)-N′-methylsulfonamido}-phenyl]-N-[2-(N″-ethylpiperidin-4-yl)eth-1-yl]acrylamide (42).
3-[2′-{(2″,6″-dichlorobenzene)-N′-ethylsulfonamido}-phenyl]-N-[2-(N″-methylpiperidin-4-yl)ethyl]acrylamide (135);
3-[2′-{(2″,6″-dichlorobenzene)-N′-ethylsulfonamido}-phenyl]-N-[4-(N″-(pyrid-4-yl)piperazinyl)phenyl]acrylamide (136);
3-[2′-{(2″,6″-dichlorobenzene)-N′-ethylsulfonamido}-phenyl]-N-[2-{N″-(pyrid-4-yl}piperidin-4-yl)eth-1-yl]acrylamide (137); and
3-trifluoromethyl-3-[2′-{(4″-chloro-2″,5″-dimethylbenzene)-N′-methylsulfonamido}-phenyl]-N-[2-{N″-(pyrid-4-yl}piperidin-4-yl)eth-1-yl]acrylamide (138).
and pharmaceutically acceptable salts thereof.

19. The compound according to claim 1 which compound is represented by formula IV: 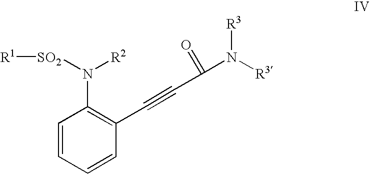
and pharmaceutically acceptable salts thereof.

20. A compound according to claim 15 which is selected from the group consisting of:
3-[2′-{(4″-chloro-2″,5″-dimethylbenzene)-N′-methylsulfonamido}-phenyl]-N-[1-(R)-1-(N″-pyrrolidinylcarbonyl)-2-(4-pyridyl)eth-1-yl]propargylamide (43);
3-[2′-{(4″-chloro-2″,5″-dimethylbenzene)-N′-methylsulfonamido}-phenyl]-N-[2-(N″-methylpiperidin-4-yl)eth-1-yl]propargylamide (44);
3-[2′-{(4″-chloro-2″,5″-dimethylbenzene)-N′-methylsulfonamido}-phenyl]-N-[1-(R)-1-(N″-piperidinylcarbonyl]eth-1-yl]propargylamide (45);
3-[2′-{(4″-chloro-2″,5″-dimethylbenzene)-N′-methylsulfonamido}-phenyl]-N-[1-(S)-1-(N″-piperidinylcarbonyl]eth-1-yl]propargylamide (46);
3-[2′-{(4″-chloro-2″,5″-dimethylbenzene)-N′-methylsulfonamido}-phenyl]-N-[2-{N″-(4-methylpyrid-2-yl)}piperidin-4-yl]eth-1-yl]propargyl-amide (47);
3-[2′-{(4″-chloro-2″,5″-dimethylbenzene)-N′-methylsulfonamido}-phenyl]-N-[1-(R)-(N″-pyrrolidinylcarbonyl)-2-(4-pyridylphen-4-yl)eth-1-yl]propargylamide (48);
3-[2′-{(4″-chloro-2″,5″-dimethylbenzene)-N′-methylsulfonamido}-phenyl]-N-[(2-N″-piperidinyl)eth-1-yl]propargylamide (49);
3-[2′-{(4″-chloro-2″,5″-dimethylbenzene)-N′-methylsulfonamido}-phenyl]-N-[(2-pyrid-4-yl)eth-1-yl]propargylamide (50);
3-[2′-{(4″-chloro-2″,5″-dimethylbenzene)-N′-methylsulfonamido}-phenyl]-N-[2-{N″-{(2-pyridyl)piperidin-4-yl}eth-1-yl]propargylamide (51);
3-[2′-{(4″-chloro-2″,5″-dimethylbenzene)-N′-methylsulfonamido}-phenyl]-N-[2-(N″-ethylpiperidin-4-yl)eth-1-yl]propargylamide (52);
3-[2′-{(4″-chloro-2″,5″-dimethylbenzene)-N′-methylsulfonamido}-phenyl]-N-[1-(S)-1-methyl-2-(N″-piperidinyl)eth-1-yl]propargylamide (53);
3-[2′-{(4″-chloro-2″,5″-dimethylbenzene)-N′-methylsulfonamido}-phenyl]-N-[2-{N″-(pyrid-4-yl}piperidin-4-yl)eth-1-yl]propargylamide (54);
3-[2′-{(4″-chloro-2″,5″-dimethylbenzene)-N′-methylsulfonamido}-phenyl]-N-[1-(R)-1-methyl-2-(N″-piperidinyl)eth-1-yl]propargylamide (55);
3-[2′-{(4″-chloro-2″,5″-dimethylbenzene)-N′-methylsulfonamido}-phenyl]-N-[2-{N″-(2-methylpyrid-4-yl}piperidin-4-yl)eth-1-yl]propargylamide (56);
3-[2′-{(4″-chloro-2″,5″-dimethylbenzene)-N′-methylsulfonamido}-phenyl]-N-[1-(S)-1-methyl-2-(4-methylpiperazin-1-yl)eth-1-yl]propargylamide (57);
3-[2′-{(4″-chloro-2″,5″-dimethylbenzene)-N′-methylsulfonamido}-phenyl]-N-[1-(R)-1-methyl-2-(4-methylpiperazin-1-yl)eth-1-yl]propargylamide (58);
3-[2′-{(4″-chloro-2″,5″-dimethylbenzene)-N′-methylsulfonamido}-phenyl]-N-methyl-N-2-(N″-methylpiperidin-4-yl)eth-1-yl]propargylamide (59);
3-[2′-{(2″,3″-dichlorobenzene)-N′-methylsulfonamido}phenyl]-N-methyl-N-2-[(N″-methylpiperidin-4-yl)eth-1-yl]propargylamide (60);
3-[2′-{(2″,5″-dimethyl-4″-chlorobenzene)-N′-methylsulfonamido}-phenyl]-N-[(α-(R,S)-methoxycarbonyl)benzyl]propargylamide (61);
3-[2′-{(2″,3″-dichlorobenzene)-N′-methylsulfonamido}phenyl]-N-N-[(α-(R,S)-methoxycarbonyl)benzyl]propargylamide (62); and
3-[2′-{(4″-chloro-2″,5″-dimethylbenzene)-N′-methylsulfonamido}-phenyl]-N-[2-(N″-ethylpiperidin-4-yl)eth-1-yl]propargylamide (63)
and pharmaceutically acceptable salts thereof.

21. The compound according to claim 1 which compound is represented by formula Va: 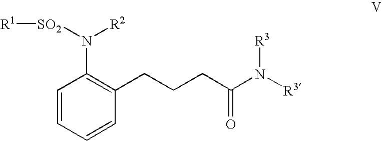
and pharmaceutically acceptable salts thereof.

22. A compound according to claim 15 which is selected from the group consisting of:
3-[2′-{(2″,5″-dimethyl-4″-chlorobenzene)-N′-methylsulfonamido}-phenyl]-N-[(α-(R,S)-methoxycarbonyl)benzyl]butyramide (64); and
3-[2′-{(2″,3″-dichlorobenzene)-N′-methylsulfonamido}phenyl]-N-N-[(α-(R,S)-methoxycarbonyl)benzyl]butyramide (65);
3-[2′-{(2″,5″-dimethyl-4″-chlorobenzene)-N′-methylsulfonamido}phenyl-N-methyl-N-[2-(N″-methylpiperidin-4-yl)ethyl]butyramide (139)
and pharmaceutically acceptable slats thereof.

23. (canceled)

24. (canceled)

25. A method for treating or palliating adverse symptoms mediated at least in part by the presence or secretion of bradykinin in a mammal which comprises administering to said mammal a therapeutically effective amount of a pharmaceutical composition according to claim 29.

26. (canceled)

27. A method for treating or ameliorating pain, hyperalgesia, hyperthermia and/or edema in mammals mediated at least in part by the release of bradykinin in such mammals which comprises a therapeutically effective of a pharmaceutical composition according to claim 29.

28. The method according to claim 27 wherein said treating or ameliorating adverse symptoms mediated at least in part by the release of bradykinin arises from burns, perioperative pain, migraine, shock, central nervous system injury, asthma, rhinitis, premature labor, inflammatory arthritis, inflammatory bowel disease or neuropathic pain.

29. A pharmaceutical composition comprising a pharmaceutically acceptable carrier and a therapeutically effective amount of a compound according to any of claim 1.

30. A method for treating or palliating adverse symptoms mediated at least in part by the presence or secretion of bradykinin in a mammal which comprises administering to said mammal a therapeutically effective amount of a compound according to any of claim 1.

31. A method for treating or ameliorating pain, hyperalgesia, hyperthermia and/or edema in mammals mediated at least in part by the release of bradykinin in such mammals which comprises a therapeutically effective of a compound according to any of claim 1.
